(19) United States
(12) Patent Application Publication
(10) Pub. No.: US 2026/0193624 A1
(43) Pub. Date: Jul. 9, 2026

(54) ENZYMES AND USES THEREOF

(71) Applicant: SAMSARA ECO PTY LIMITED, Crows Nest, New South Wales (AU)

(72) Inventors: Colin John JACKSON, Kingston, Australian Capital Territory (AU); Vanessa VONGSOUTHI, Gungahlin, Australian Capital Territory (AU); Matthew Arthur SPENCE, Kaleen, Australian Capital Territory (AU)

(21) Appl. No.: 19/106,701

(22) PCT Filed: Aug. 25, 2023
(86) PCT No.: PCT/AU2023/050828

§ 371 Date: Feb. 26, 2025

(30) Foreign Application Priority Data
Aug. 26, 2022 (AU) 2022902457

(51) Int. Cl. C12N 9/16; C08J 11/10; C08J 11/16; C08J 11/24; C12P 7/04; C12P 7/44; C12P 7/62

(52) U.S. Cl. CPC C12N 9/16; C08J 11/105; C08J 11/16; C08J 11/24; C12P 7/04; C12P 7/44; C12P 7/62; C08J 2367/02

(57) ABSTRACT

The present disclosure relates generally to polypeptides having esterase activity, wherein the esterase activity is capable of converting a monoester terephthalate to terephthalic acid and an alcohol; diester terephthalate to a monoester terephthalate and an alcohol; or diester terephthalate to terephthalic acid and an alcohol, and their methods of use.

This application claims priority to Australian Provisional Application No. 2022902457 entitled “Enzymes and uses thereof” filed 26 Aug. 2022, the contents of which are incorporated herein by reference in their entirety.

### FIELD OF THE ART

The present invention relates to novel synthetic enzymes, more particularly to recombinant enzymes that catalyse the hydrolysis of mono- and di-terephthalic acid esters and uses thereof.

### BACKGROUND

All references, including any patent or patent application cited in this specification are hereby incorporated by reference to enable full understanding of the invention. Nevertheless, such references are not to be read as constituting an admission that any of these documents forms part of the common general knowledge in the art, in Australia or in any other country.

Global industrialization has had significant environmental impact, not least of which is an increase in the manufacture and reliance on plastic and plastic products. Whilst there is a growing effort to find suitable and environmentally sustainable alternatives to plastics, including their manufacture and disposal, such products remain a significant problem and contribute to a vast majority of environmental pollutants. One of the major contributors to this problem is polyethylene terephthalate (PET) and its waste products, millions of tons of which are produced globally every year. The environmental significance of this problem is attributed, at least in part, to the chemical nature of plastics, in particular PET based products, as they do not readily decompose in nature.

Approaches to deal with the problem of plastic waste products have typically included incineration, disposal in landfill and mechanical disintegration. However, these approaches also have significant environmental impact. For instance, incineration of plastics produces potentially harmful by-products that are released into the atmosphere; the decomposition rate of plastics in landfill is typically very slow and there is a risk that toxic materials will leach into groundwater; and mechanical disintegration is relatively expensive and inefficient and there is often limited use for its by-products.

More recently, biological (enzymatic) degradation of plastics has been considered as an alternative approach to reducing plastic waste accumulation. This approach includes the use of PETases, an esterase class of enzyme that catalyze the hydrolysis of PET to the monomeric mono-2-hydroxyethyl terephthalate (MHET), and the resultant MHET is further broken down by the action of MHETases to terephthalic acid and ethylene glycol. Terephthalic acid is the precursor to polyester PET.

Whilst enzymatic degradation of plastics is an attractive alternative to alleviating the environmental impact of plastic waste products and their disposal, it has not yet seen widespread adoption, including because of its relative inefficiency, slow rate of enzymatic degradation and low levels of enzyme expression in common industrial host organisms. Hence, there remains an urgent need for improved methods and reagents for the enzymatic degradation of plastics.

### SUMMARY OF THE INVENTION

In one aspect disclosed herein, there is provided a polypeptide having esterase activity, wherein the esterase activity is capable of converting a monoester terephthalate to terephthalic acid and an alcohol; diester terephthalate to a monoester terephthalate and an alcohol; or diester terephthalate to terephthalic acid and an alcohol; wherein the polypeptide comprises an amino acid sequence selected from the group consisting of: amino acids 5-261 of SEQ ID NO:2 or an amino acid sequence having at least 85% sequence identity thereto; amino acids 5-261 of SEQ ID NO:3 or an amino acid sequence having at least 77% sequence identity thereto; amino acids 5-261 of SEQ ID NO: 4 or an amino acid sequence having at least 75% sequence identity thereto; amino acids 5-261 of SEQ ID NO: 5 or an amino acid sequence having at least 95% sequence identity thereto; and amino acids 5-261 of SEQ ID NO: 6 or an amino acid sequence having at least 96% sequence identity thereto, and wherein the polypeptide is not SEQ ID NO:1 or SEQ ID NO:12.

In an embodiment, the polypeptide comprises the amino acid sequence of amino acids 5-261 of SEQ ID NO: 6, or an amino acid sequence having at least 96% sequence identity thereto.

In an embodiment, the polypeptide comprises the amino acid sequence of amino acids 5-261 of SEQ ID NO: 7, or an amino acid sequence having at least 97% identity thereto.

In an embodiment, the polypeptide comprises the amino acid sequence of amino acids 5-261 of SEQ ID NO: 8, or an amino acid sequence having at least 96% identity thereto.

In an embodiment, the polypeptide comprises the amino acid sequence of amino acids 5-261 of SEQ ID NO: 9, or an amino acid sequence having at least 97% identity thereto.

In an embodiment, the polypeptide comprises the amino acid sequence of amino acids 5-261 of SEQ ID NO: 10, or an amino acid sequence having at least 98% identity thereto.

In an embodiment, the polypeptide comprises the amino acid sequence of amino acids 5-261 of SEQ ID NO: 11, or an amino acid sequence having at least 98% identity thereto.

In another embodiment, the polypeptide comprises the amino acid sequence of SEQ ID NO:119.

In another aspect there is provided a polypeptide having esterase activity, wherein the esterase activity is capable of converting a monoester terephthalate to terephthalic acid and an alcohol; a diester terephthalate to a monoester terephthalate and an alcohol; or a diester terephthalate to terephthalic acid and an alcohol, wherein the polypeptide comprises an amino acid sequence of SEQ ID NO:119.

In another aspect disclosed herein, there is provided a method for the enzymatic hydrolysis of monoester terephthalate and/or diester terephthalate generated as by-product of degradation of PET, wherein the monoester terephthalate is a mono-C₁-C₁₀ alkyl terephthalate, optionally substituted with benzyl, and wherein the mono-ester terephthalate is not mono-(2-hydroxyethyl)terephthalate (MHET); the diester terephthalate is a di-C₁-C₁₀ alkyl terephthalate, optionally substituted with benzyl; the method comprising exposing the monoester terephthalate and/or diester terephthalate to a polypeptide having esterase activity, under conditions sufficient to enable the polypeptide to convert the: monoester terephthalate to terephthalic acid and an alcohol; diester terephthalate to a monoester terephthalate and an alcohol; or diester terephthalate to terephthalic acid and an alcohol.

In an embodiment, the polypeptide comprises the amino acid sequence of SEQ ID NO:1 or an amino acid sequence having at least 70% sequence identity to SEQ ID NO: 1.

In an embodiment, the monoester terephthalate is selected from a group consisting of monobenzyl terephthalate (MBZT), monohexyl terephthalate, monoheptyl terephthalate (MHPT) and monooctyl terephthalate (MOCT). In a preferred embodiment, the monoester terephthalate is MBZT or MOCT. In one embodiment, the polypeptide comprises the amino acid sequence of amino acids 5-261 of any one of SEQ ID NOs: 9-11 or an amino acid sequence that has at least 70% sequence identity to any of the foregoing.

In an embodiment, the monoester terephthalate is selected from a group consisting of dibenzyl terephthalate (DBZT), dihexyl terephthalate (DHXT), diheptyl terephthalate (DHPT) and dioctyl terephthalate (DOCT). In a preferred embodiment, the monoester terephthalate is DBZT or DOCT. In one embodiment, the polypeptide comprises the amino acid sequence of amino acids 5-261 of any one of SEQ ID NOs: 6-8 or an amino acid sequence that has at least 70% sequence identity to any of the foregoing.

In an embodiment, the monoester terephthalate and diester terephthalate is generated by the hydrolysis or degradation of a polyethylene terephthalate (PET).

The present disclosure also extends to a composition comprising the polypeptide as described herein.

The present disclosure also extends to a nucleic acid sequence encoding the polypeptide described herein.

The present disclosure also extends to an expression vector comprising the nucleic acid sequence described herein.

The present disclosure also extends to a host cell comprising the nucleic acid sequence or the expression vector described herein.

In another aspect, the present disclosure provides a method of producing a polypeptide having esterase activity, the method comprising
i) providing the polynucleotide described herein;
ii) expressing the polynucleotide in a host cell under conditions sufficient to allow the host cell to produce the polypeptide; and
iii) collecting the polypeptide produced by the host cell in ii).

The present disclosure also extends to a method of degrading a plastic product comprising a polyester, the method comprising contacting the plastic product with the polypeptide, the composition, or the host cell described herein, under conditions sufficient to enable the polypeptide to degrade the plastic product. In an embodiment, the plastic product comprises the polyester polyethylene terephthalate (PET).

The present disclosure also extends to a composition comprising the terephthalic acid and/or alcohol recovered by the method disclosed herein.

In another aspect, there is provided a host cell genetically modified to express the polypeptide described herein.

In another aspect, there is provided a method of producing a plastic product using the composition of the terephthalic acid and alcohol generated by the method disclosed herein.

### BRIEF DESCRIPTION OF THE DRAWINGS

FIG. 1 shows a schematic representation of the enzymatic conversion of (i) monoester terephthalate to terephthalic acid and an alcohol; (ii) diester terephthalate to a monoester terephthalate and an alcohol; and (iii) diester terephthalate to terephthalic acid and an alcohol. In this schematic, X is C₁-C₁₀ and the reformed C₁-C₁₀ mono-alcohol is represented by X—OH.

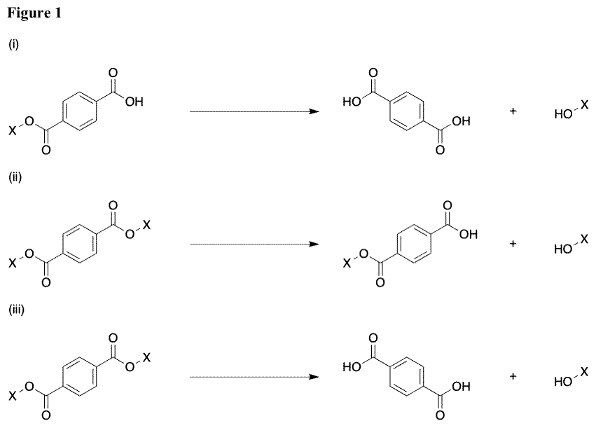

FIG. 2 demonstrates the DOCTase activity of polypeptides having the amino acid sequences of SEQ ID NOs 69-118 (from left to right) disclosed herein, as determined by the combined concentration of monomer equivalent hydrolysis products MOCT and TPA (mg/mL), as determined by U/HPLC (as represented on the Y-axis). The SEQ ID NOs are provided on the X-axis.

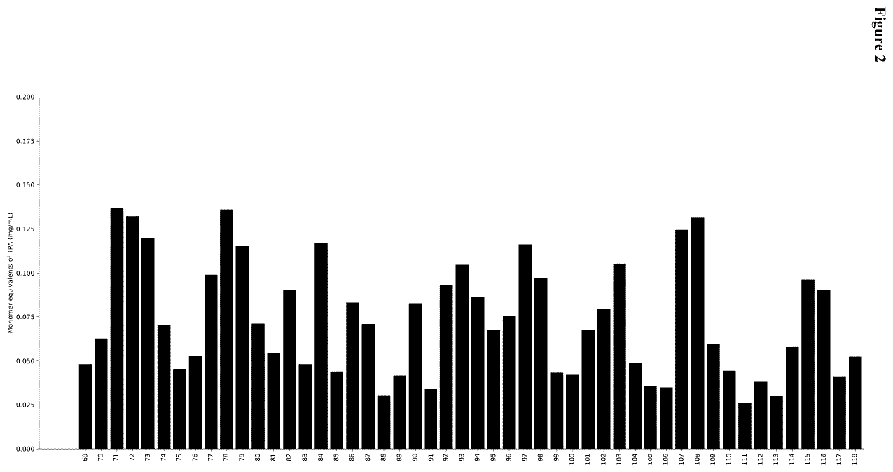

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article, unless explicitly stated otherwise. By way of example, “an element” means one element or more than one element.

As used herein, the term “about” refers to a quantity, level, value, dimension, size, or amount that varies by as much as 10% (e.g, by 10%, 9%, 8%, 7%, 6%, 5%, 4%, 3%, 2% or 1%) to a reference quantity, level, value, dimension, size, or amount.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

While enzymatic conversion of PET to monomeric mono-2-hydroxyethyl terephthalate (MHET) and Bis-(2-hydroxyethyl) terephthalic acid (BHET) by PETase enzymes and the enzymatic hydrolysis of MHET to terephthalate/TPA by MHETases are known (MHETase was originally discovered alongside PETase in the bacterium Ideonella sakaiensis; the two enzymes enable the bacterium to live on the plastic PET as a carbon source; Yoshida et al. (2016) Science 351: 1196)), enzymes capable of degrading or hydrolysing other plastics or PET intermediates, including terephthalate esters (in particular di- and mono-ester terephthalates other than MHET and BHET, see for example Ion et al., 2021 Catalysis Today 366:177), remain to be identified. The present disclosure is predicated, at least in part, on the inventors' identification of esterases that have surprising hydrolase/esterase activity on mono- and di-terephthalate esters of PET, such as mono- and di-C₁-C₁₀ alkyl terephthalates, in particular mono- and di-C₆-C₁₀ alkyl terephthalates.

Thus, in an aspect disclosed herein, there is provided a polypeptide having esterase activity, wherein the esterase activity is capable of converting a monoester terephthalate to terephthalic acid and an alcohol; diester terephthalate to a monoester terephthalate and an alcohol; or diester terephthalate to terephthalic acid and an alcohol; wherein the polypeptide comprises an amino acid sequence selected from the group consisting of: amino acids 5-261 of SEQ ID NO:2 or an amino acid sequence having at least 85% sequence identity thereto; amino acids 5-261 of SEQ ID NO:3 or an amino acid sequence having at least 77% sequence identity thereto; amino acids 5-261 of SEQ ID NO: 4 or an amino acid sequence having at least 75% sequence identity thereto; amino acids 5-261 of SEQ ID NO: 5 or an amino acid sequence having at least 95% sequence identity thereto; and amino acids 5-261 of SEQ ID NO: 6 or an amino acid sequence having at least 96% sequence identity thereto, wherein the polypeptide is not SEQ ID NO:1 or SEQ ID NO:12.

In an embodiment, the monoester terephthalate is a mono-C1-C10 alkyl terephthalate, optionally substituted with benzyl, and the diester terephthalate is a di-C1-C10 alkyl terephthalate, optionally substituted with benzyl. In a preferred embodiment, the monoester terephthalate is selected from a group consisting of monobenzyl terephthalate (MBZT), monohexyl terephthalate (MHXT), monoheptyl terephthalate (MHPT) and monooctyl terephthalate (MOCT), the diester terephthalate is selected from a group consisting of dibenzyl terephthalate (DBZT), dihexyl terephthalate(DHXT), diheptyl terephthalate (DHPT) and dioctyl terephthalate (DOCT).

Reference to terephthalic acid, as used herein, also extends to salts of terephthalic acid. Thus, in an embodiment, the terephthalic acid is a salt of terephthalic acid. In an embodiment, the salt of terephthalic acid is a metal alkali salt. In another embodiment, the terephthalic acid produced is a terephthalic acid disodium salt.

By “at least 85%” is meant that the polypeptide shares at least 85%, preferably at least 87%, preferably at least 88%, preferably at least 90%, preferably at least 92%, preferably at least 94%, preferably at least 95%, preferably at least 96%, preferably at least 97%, preferably at least 98%, or more preferably at least 99% sequence identity to SEQ ID NO:2 or to amino acids 5-261 of SEQ ID NO: 2. As the polypeptide described herein is a synthetic polypeptide, it is to be understood that, in this context, “at least 85%” can include 100% sequence identity across the entire sequence of SEQ ID NO:2 or across the entire amino acid sequence of amino acids 5-261 of SEQ ID NO:2.

By “at least 77%” is meant that the polypeptide shares at least 77%, preferably at least 80%, preferably at least 85%, preferably at least 90%, preferably at least 92%, preferably at least 94%, preferably at least 95%, preferably at least 96%, preferably at least 97%, preferably at least 98%, or more preferably at least 99% sequence identity to SEQ ID NO:3 or to amino acids 5-261 of SEQ ID NO: 3. As the polypeptide described herein is a synthetic polypeptide, it is to be understood that, in this context, “at least 77%” can include 100% sequence identity across the entire sequence of SEQ ID NO:3 or across the entire sequence of amino acids 5-261 of SEQ ID NO:3.

By “at least 75%” is meant that the polypeptide shares at least 75%, preferably at least 80%, preferably at least 85%, preferably at least 90%, preferably at least 92%, preferably at least 94%, preferably at least 95%, preferably at least 96%, preferably at least 97%, preferably at least 98%, or more preferably at least 99% sequence identity to SEQ ID NO:4 or to amino acids 5-261 of SEQ ID NO: 4. As the polypeptide described herein is a synthetic polypeptide, it is to be understood that, in this context, “at least 85%” can include 100% sequence identity across the entire sequence of SEQ ID NO:4 or across the entire sequence of amino acids 5-261 of SEQ ID NO:4.

By “at least 95%” is meant that the polypeptide shares at least 95%, preferably at least 96%, preferably at least 97%, preferably at least 98%, or more preferably at least 99% sequence identity to SEQ ID NO:5 or to amino acids 5-261 of SEQ ID NO: 5. As the polypeptide described herein is a synthetic polypeptide, it is to be understood that, in this context, “at least 95%” can include 100% sequence identity across the entire sequence of SEQ ID NO:5 or across amino acids 5-261 of SEQ ID NO:5.

By “at least 96%” is meant that the polypeptide shares at least 96%, preferably at 97%, preferably at least 98%, or more preferably at least 99%, sequence identity to SEQ ID NO: 6 or amino acids 5-261 of SEQ ID NO:6. As the polypeptide described herein is a synthetic polypeptide, it is to be understood that, in this context, “at least 95%” can include 100% sequence identity across the entire sequence of SEQ ID NO:6 or across the entire sequence of amino acids 5-261 of SEQ ID NO:6.

In an embodiment, the polypeptide comprises an amino acid sequence of amino acids 5-261 of SEQ ID NO:2 or an amino acid sequence having at least 85% sequence identity thereto. In another embodiment the polypeptide consists of the amino acid sequence of SEQ ID NO: 2.

In an embodiment, the polypeptide comprises an amino acid sequence of amino acids 5-261 of SEQ ID NO:3 or an amino acid sequence having at least 77% sequence identity thereto. In another embodiment the polypeptide consists of the amino acid sequence of SEQ ID NO: 3.

In an embodiment, the polypeptide comprises an amino acid sequence of amino acids 5-261 of SEQ ID NO: 4 or an amino acid sequence having at least 75% sequence identity thereto. In another embodiment the polypeptide consists of the amino acid sequence of SEQ ID NO: 4.

In an embodiment, the polypeptide comprises an amino acid sequence of amino acids 5-261 of SEQ ID NO: 5 or an amino acid sequence having at least 95% sequence identity thereto. In another embodiment the polypeptide consists of the amino acid sequence of SEQ ID NO: 5.

In an embodiment, the polypeptide comprises an amino acid sequence of amino acids 5-261 of SEQ ID NO: 6 or an amino acid sequence having at least 96% sequence identity thereto.

In another embodiment the polypeptide consists of the amino acid sequence of SEQ ID NO: 6.

In one embodiment the polypeptide comprises the amino acid sequence of amino acids 5-261 of SEQ ID NO: 7. In one embodiment the polypeptide comprises the amino acid sequence amino acids 5-261 of SEQ ID NO: 8. In one embodiment the polypeptide comprises the amino acid sequence of amino acids 5-261 of SEQ ID NO: 9. In one embodiment the polypeptide comprises the amino acid sequence of amino acids 5-261 of SEQ ID NO: 10. In one embodiment, the polypeptide comprises the amino acid sequence of amino acids 5-261 of SEQ ID NO: 11.

In another embodiment the polypeptide consists or consists essentially of the amino acid sequence of SEQ ID NO: 6. In one embodiment the polypeptide consists or consists essentially of the amino acid sequence of SEQ ID NO: 7. In one embodiment the polypeptide consists or consists essentially of SEQ ID NO: 8. In one embodiment the polypeptide consists or consists essentially of SEQ ID NO: 9. In one embodiment the polypeptide consists or consists essentially of SEQ ID NO: 10. In one embodiment, the polypeptide consists or consists essentially of SEQ ID NO: 11.

In an embodiment, the polypeptide comprises the amino acid sequence of amino acids 5-260 of any one of SEQ ID NOs: 1-118.

In an embodiment, the polypeptide comprises the amino acid sequence of SEQ ID NO: 119. In an embodiment, the amino acid residue at position X of SEQ ID NO:119 is selected from the amino acid residue at a corresponding position of any one of SEQ ID NOs: 1-118.

In another embodiment, the polypeptide further comprises an N-terminal cellular export signal peptide or signal peptide selected from the amino acid sequence of SEQ ID NO: 120 (MAAN); SEQ ID NO: 121 (MAEN); SEQ ID NO: 122 (MQAN); SEQ ID NO: 123 (MADN); and SEQ ID NO: 124 (MQSN).

Monoester terephthalates would be familiar to persons skilled in the art. For example, as used herein the term monoester terephthalates refers to a 1,4 di-substituted benzene where the substitutions are a carboxylic acid functional group and an ester functional group.

Monoester terephthalates include mono-alkyl terephthalates. In some embodiments, the monoester terephthalate is formed through transesterification of the PET with C₁-C₁₀ mono-alcohol. In particular embodiments, the monoester terephthalate is formed through transesterification of the PET with C₆-C₁₀ mono-alcohol. In particular embodiments, the monoester terephthalate is formed through transesterification of the PET with benzyl alcohol, hexanol, heptanol or octanol.

Diester terephthalates would be familiar to persons skilled in the art. For example, as used herein the term diester terephthalates refers to a 1,4 di-substituted benzene where the substitutions are ester functional groups. Diester terephthalates include di-alkyl terephthalates. In some embodiments, the diester terephthalate is formed through transesterification of the PET with C₁-C₁₀ mono-alcohol. In particular embodiments, the diester terephthalate is formed through transesterification of the PET with C₆-C₁₀ mono-alcohol. In particular embodiments, the diester terephthalate is formed through transesterification of the PET with benzyl alcohol, hexanol, heptanol or octanol.

The present disclosure also extends to a composition comprising the polypeptide as described herein.

The present disclosure also extends to a nucleic acid sequence encoding the polypeptide described herein.

The present disclosure also extends to an expression vector comprising the nucleic acid sequence described herein.

The present disclosure also extends to a host cell comprising the nucleic acid sequence or the expression vector described herein.

In another aspect, the present disclosure provides a method of producing a polypeptide having esterase activity, the method comprising
i) providing the polynucleotide described herein;
ii) expressing the polynucleotide in a host cell under conditions sufficient to allow the host cell to produce the polypeptide; and
iii) collecting the polypeptide produced by the host cell in (ii).

In yet another aspect, there is provided a method for the enzymatic hydrolysis of monoester terephthalate and/or diester terephthalate generated as by-product of degradation of PET, wherein
a. the monoester terephthalate is a mono-C₁-C₁₀ alkyl terephthalate, optionally substituted with benzyl, and wherein the mono-ester terephthalate is not mono-(2-hydroxyethyl)terephthalate (MHET);
b. the diester terephthalate is a di-C₁-C₁₀ alkyl terephthalate, optionally substituted with benzyl;
the method comprising exposing the monoester terephthalate and/or diester terephthalate to a polypeptide having esterase activity, under conditions sufficient to enable the polypeptide to convert the:
   i. monoester terephthalate to terephthalic acid and an alcohol;
   ii. diester terephthalate to a monoester terephthalate and an alcohol; or
   iii. diester terephthalate to terephthalic acid and an alcohol.

On one embodiment, the polypeptide used in the hydrolysis of monoester terephthalate and/or diester terephthalate generated as by-product of degradation of PET comprises the amino acid sequence of SEQ ID NO:1 or an amino acid sequence having at least 70% sequence identity to SEQ ID NO: 1.

In an embodiment, the monoester terephthalate is a mono-C₆-C₁₀ alkyl terephthalate, optionally substituted with benzyl. In one embodiment, the ester is a mono-C₆ alkyl ester. In one embodiment, the ester is a mono-C₇ alkyl ester. In one embodiment, the ester is a mono-C₈ alkyl ester. In another embodiment, the ester is a mono-C₉ alkyl ester. In another embodiment, the ester is a mono-C₁₀ alkyl ester. In an embodiment, the monoester terephthalate is selected from a group consisting of monobenzyl terephthalate (MBZT), monohexyl terephthalate, monoheptyl terephthalate (MHPT) and monooctyl terephthalate (MOCT). In a preferred embodiment, the monoester terephthalate is MBZT or MOCT. In a preferred embodiment, the polypeptide comprises the amino acid sequence of any one of SEQ ID NOs: 9-11 and an amino acid sequence that has at least 70% sequence identity to any of the foregoing.

In an embodiment, the monoester terephthalate is a di-C₆-C₁₀ alkyl terephthalate, optionally substituted with benzyl. In one embodiment, the ester is a di-C₆ alkyl ester. In one embodiment, the ester is a di-C₇ alkyl ester. In one embodiment, the ester is a di-C₈ alkyl ester. In another embodiment, the ester is a di-C₉ alkyl ester. In another embodiment, the ester is a di-C₁₀ alkyl ester. In an embodiment, the diester terephthalate is selected from a group consisting of dibenzyl terephthalate (DBZT), dihexyl terephthalate (DHXT), diheptyl terephthalate (DHPT) and dioctyl terephthalate (DOCT). In a preferred embodiment, the monoester terephthalate is DBZT or DOCT. In a preferred embodiment, the polypeptide comprises the amino acid sequence of any one of SEQ ID NOs: 6-8 and an amino acid sequence that has at least 70% sequence identity to any of the foregoing.

In an embodiment, the monoester terephthalate and diester terephthalate is generated by the hydrolysis or degradation of a polyethylene terephthalate (PET). In another embodiment, the monoester terephthalate and diester terephthalate is generated by a process comprising exposing PET to sodium hydroxide, and/or contacting the PET to an esterase. In a preferred embodiment, the monoester terephthalate and diester terephthalate is generated by a process comprising subjecting the PET to base-catalysed transesterification with a C₆-C₁₀ mono-alcohol; and/or contacting the PET to an esterase. In a preferred embodiment, the C₆-C₁₀ mono-alcohol is a benzyl alcohol, an octanol or a heptanol. In yet another preferred embodiment, the C₆-C₁₀ mono-alcohol is 1-octanol.

The present disclosure also extends to a method of degrading a plastic product comprising a polyester, the method comprising contacting the plastic product with the polypeptide, the composition, or the host cell described herein, under conditions sufficient to enable the polypeptide to degrade the plastic product.

In an embodiment, the polyester is selected from the group consisting of polylactic acid (PLA), polytrimethylene terephthalate (PTT), polybutylene terephthalate (PBT), polyethylene isosorbide terephthalate (PEIT), polyethylene terephthalate (PET) polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate (PEA), poly(glycolic acid) (PGA), poly(lactic-co-glycolic acid) (PLGA) and combinations of any of the foregoing. In an embodiment the polyester is polyethylene terephthalate (PET).

The present disclosure also extends to a composition comprising the terephthalic acid and/or ethylene glycol recovered by the method disclosed herein.

In another aspect, there is provided a host cell genetically modified to express the polypeptide described herein.

In another aspect, there is provided a method of producing a plastic product using the composition of the terephthalic acid and ethylene glycol generated by the method disclosed herein.

The term “wild-type” is used herein to denote a naturally-occurring isoform of a polypeptide; that is, as it appears in nature. The term “extant” is used to denote naturally-occurring isoforms of a polypeptide found in organisms or species still in existence (i.e. not extinct).

Examples of extant cutinases would be familiar to the persons skilled in the art. Examples of extant cutinases include, but are not limited to: Leaf-branch compost cutinase (LCC; Accession number: G9BY57), TfCut2 (cutinase from Thermobifida fusca; Accession number: E5BBQ3), E9LVH9 (Cut2 from Thermobifida cellulosilytica) and E5BBQ2 (Cut.1-KW3 cutinase from Thermobifida fusca).

Herein, the terms “peptide”, “polypeptide”, “protein”, “enzyme” are to be understood as referring to a chain of amino acids linked by peptide bonds, irrespective of the number of amino acids forming said chain. The amino acids are typically represented by their one-letter or three-letters code, according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gln); R: arginine (Arg); S: serine (Ser); T: threonine (Thr); V: valine (Val); W: tryptophan (Trp) and Y: tyrosine (Tyr).

The term “hydrolase” as used herein typically refers to an enzyme which belongs to a class of hydrolases classified as EC 3 according to Enzyme Nomenclature that catalyzes the hydrolysis of chemical bonds, including ester bonds.

The term “esterase” as used herein typically refers to a hydrolase enzyme which is classified as EC 3.1 according to Enzyme Nomenclature that catalyzes the hydrolysis of ester bonds to produce an acid and an alcohol. The term “cutinase” refers to a serine esterase enzyme which is classified as EC 3.1.1.74 according to Enzyme Nomenclature, which catalyses the hydrolysis of cutin polymers (a polyester composed of hydroxy and hydroxyepoxy fatty acids) into cutin monomers.

The term “PETase” as used herein, typically refers to an esterase enzyme, which is classified as EC 3.1.1.101 according to Enzyme Nomenclature that catalyzes the hydrolysis of polyethylene terephthalate (PET) plastic to monomeric mono-2-hydroxyethyl terephthalate (MHET).

Herein, the polypeptide of the present invention is understood to have hydrolase or esterase activity, having capability to catalyse the hydrolysis of mono- and di-terephthalic acid esters.

As noted by Palm et al. (2019, Nat. Comms. 10:1717), two recently discovered bacterial enzymes that specifically degrade polyethylene terephthalate (PET) represent a promising solution to an otherwise environmentally burdensome polyester containing product. First, Ideonella sakaiensis PETase, a structurally well-characterized α/β-hydrolase fold enzyme, converts PET to mono-(2-hydroxyethyl) terephthalate (MHET). MHETase, the second key enzyme, hydrolyzes MHET to terephthalate and ethylene glycol (Palm et al. (2019, Nat. Comm., 10:1717), Sagong et al. (2020, ACS Catal. 10:4805) and Yoshida et al. (2016, Science, 352(6278):1196).

The terms “mutant” and “variant” may be used interchangeably herein to refer to a polypeptide comprising an amino acid sequence that is derived from SEQ ID NO:1 and further comprising a modification or alteration (e.g., a substitution, insertion, and/or deletion), at one or more (e.g., several) positions and having enhanced esterase activity in catalysing the hydrolysis of mono- and di-terephthalic acid esters, when compared to extant PETases or cutinases having PETase activity or the polypeptide of SEQ ID NO:1. Such variants may be obtained by various techniques well known in the art, illustrative examples of which include site-directed mutagenesis, random mutagenesis and synthetic oligonucleotide construction. The terms “modification”, “alteration”, “substitution” and the like, as used herein in relation to an amino acid residue or position, typically mean that the amino acid in the particular position has been modified compared to the amino acid of the wild-type or parent polypeptide.

Suitable substitutions may include the replacement of an amino acid residue by another selected from the naturally-occurring standard 20 amino acid residues, rare naturally occurring amino acid residues (e.g., hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid, ornithine, norleucine, norvaline), and non-naturally occurring amino acid residue, often made synthetically, (e.g., cyclohexyl-alanine). Preferably, the substitution comprises the replacement of an amino acid residue by another selected from the naturally-occurring standard 20 amino acid residues (G, P, A, V, L, I, M, C, F, Y, W, H, K, R, Q, N, E, D, S and T). The modification or alteration may be identified herein using the following terminology: Y197V denotes that amino acid residue Tyrosine (Y) at position 197 of the parent polypeptide sequence is substituted for a Valine (V). Y197V/I/M denotes that amino acid residue Tyrosine (Y) at position 197 of the parent sequence may be substituted for one of the following amino acids: Valine (V), Isoleucine (I), or Methionine (M). The substitution can be a conservative or non-conservative substitution. Examples of conservative substitutions will be familiar to persons skilled in the art, illustrative examples of which include substitutions within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

The positions disclosed in the present application are numbered by reference to the amino acid sequence set forth the recited SEQ ID NOs. In this context, the term “corresponding to”, when used in reference to an amino acid position, is intended to mean an amino acid position in a polypeptide sequence when that position is aligned with the equivalent or corresponding position in the sequence set forth in a reference sequence. For example, the amino acid residue at position 5 of SEQ ID NO:6 (amino acid P/proline) would correspond to the amino acid residue at position 4 of SEQ ID NO:4 (amino acid P/proline) of SEQ ID NO:116. In another example, the amino acid residue at position 14 of SEQ ID NO:19 (amino acid N/asparagine) would correspond to the amino acid residue at position 13 of SEQ ID NO:113 (amino acid E/glutamic acid). In yet another example, in the context of SEQ ID NO:119, the amino acid residue at position 4 of SEQ ID NO:119 (amino acid P/proline) would correspond to the amino acid residue at position 12 of SEQ ID NO:44 (amino acid P/proline); and the amino acid residue at position 3 of SEQ ID NO:119 would correspond to the amino acid residue at position 11 of SEQ ID NO:44 (amino acid A/alanine). In a further example, in the context of SEQ ID NO:119, the amino acid residue at position 3 of SEQ ID NO:119 would correspond to the amino acid residue at position 10 of SEQ ID NO:105 (amino acid D/aspartic acid).

As used herein, the term “sequence identity” or “identity” refers to the number (or fraction expressed as a percentage %) of matches (identical amino acid residues) between two polypeptide sequences. In a preferred embodiment, the sequence identity is determined by comparing the sequences when aligned so as to maximize overlap and identity while minimizing sequence gaps. Sequence identity may be determined using any of a number of mathematical global or local alignment algorithms known to persons skilled in the art, depending on the length of the two sequences. Sequences of similar lengths may be aligned using a global alignment algorithms (e.g., Needleman and Wunsch algorithm; Needleman and Wunsch, 1970), which aligns the sequences optimally over the entire length, while sequences of substantially different lengths are preferably aligned using a local alignment algorithm (e.g., Smith and Waterman algorithm (Smith and Waterman, 1981) or Altschul algorithm (Altschul et al., 1997; Altschul et al., 2005)). Alignment for the purposes of determining percent amino acid sequence identity can be achieved by any means available to persons skilled in the art, illustrative examples of which include publicly available computer software, such as is available at http://blast.ncbi.nlm.nih.gov/ or http://www.ebi.ac.uk/Tools/emboss/). Persons skilled in the art can readily determine appropriate parameters for measuring alignment, including any algorithms needed to achieve maximal alignment over the full length of the sequences being compared. As used herein, % sequence identity typically refers to values generated using pairwise sequence alignment that creates an optimal global alignment of two sequences (e.g., using the Needleman-Wunsch algorithm), where all search parameters are set to default values, e.g., Scoring matrix=BLOSUM62, Gap open=10, Gap extend=0.5, End gap penalty=false, End gap open=10 and End gap extend=0.5.

The term “recombinant”, as used herein, typically refers to a nucleic acid construct, a vector, a polypeptide or a cell produced by genetic engineering.

The term “expression”, as used herein, typically refers to any step involved in the production of a polypeptide, such as by transcription, post-transcriptional modification, translation, post-translational modification, and secretion.

The term “expression cassette” denotes a nucleic acid construct comprising a coding region, and suitably a regulatory region to which the coding region is operably linked.

The term “expression vector” typically means a DNA or RNA molecule that comprises an expression cassette. The expression vector may be a linear or circular double stranded DNA molecule.

The term “polymer”, as used herein, typically refers to a chemical compound or a mixture of compounds whose structure is made up of multiple monomers (repeat units) linked by covalent chemical bonds. Within the context of the invention, the term polymer includes natural or synthetic polymers, constituted of a single type of repeat unit (i.e., homopolymers) or of a mixture of different repeat units (i.e., copolymers or heteropolymers).

As used herein, the terms “polyester containing material”, “polyester containing product” and the like are to be understood as refers to a product, such as plastic product, comprising at least one polyester in crystalline, semi-crystalline or totally amorphous form. The polyester containing material may refer to any item made from at least one plastic material, such as plastic sheet, tube, rod, profile, shape, film, massive block, fiber, textiles, etc., which contains at least one polyester, and possibly other substances or additives, such as plasticizers, mineral or organic fillers. In an embodiment, the polyester containing material is a textile or fabric comprising at least one polyester containing fiber. In another embodiment, the polyester containing material is a plastic compound, or plastic formulation, in a molten or solid state, suitable for making a plastic product.

Suitable polyesters will be familiar to persons skilled in the art, illustrative examples of which include polylactic acid (PLA), polyethylene terephthalate (PET), polytrimethylene terephthalate (PTT), polybutylene terephthalate (PBT), polyethylene isosorbide terephthalate (PEIT), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), and poly(ethylene adipate) (PEA). Thus, in an embodiment, the polyester is selected from the group consisting of polylactic acid (PLA), polyethylene terephthalate (PET), polytrimethylene terephthalate (PTT), polybutylene terephthalate (PBT), polyethylene isosorbide terephthalate (PEIT), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA) and combinations of any of the foregoing.

Suitable methods of determining or measuring the esterase/hydrolase activity of a polypeptide will be familiar to persons skilled in the art, an illustrative example of which is described elsewhere herein. Other illustrative examples are described in Palm et al. (2019, Nat. Comm., 10:1717), Sagong et al. (2020, ACS Catal. 10:4805) and Yoshida et al. (2016, Science, 352(6278):1196), the contents of which are incorporated herein by reference in their entirety. Another method useful for determining or measuring the esterase/hydrolase activity of a polypeptide is by measuring the amount of terephthalic acid or monoester terephthalate produced using Analytical High Performance Liquid Chromatography (HPLC).

The esterase activity of the novel engineered polypeptide and variants having esterase activity may be assigned an absolute value or a value relative to the esterase activity of a comparator. In an embodiment, the esterase activity is measured as the rate of monomers and/or oligomers (e.g., in mg or mol) released per hour and per mg or mol of enzyme under suitable conditions of temperature, pH and buffer. In an embodiment of the invention, the rate of monomers and/or oligomers released per hour is in the range of from about 1 mol/h/mol enzyme to about 500 mol/h/mol enzyme. In another embodiment, the rate of monomers and/or oligomers (e.g., in mg) released per hour is in the range of from about 50 mol/h/mol enzyme to about 400 mol/h/mol enzyme. In yet another embodiment, the rate of monomers and/or oligomers (e.g., in mg) released per hour is in the range of from about 100 mol/h/mol enzyme to about 350 mol/h/mol enzyme.

Advantageously, the polypeptide described herein may be able to catalyse the hydrolysis of mono- and di-terephthalic acid esters at least in a range of temperatures from about 10° C. to about 80° C., from about 20° C. to about 80° C., from about 30° C. to about 80° C., from about 30° C. to about 70° C., preferably from about 40° C. to about 70° C., preferably from about 50° C. to about 70° C., or even more preferably at about 50° C. to about 60° C. In an embodiment, the polypeptide described herein exhibits esterase activity at a temperature from about 10° C. to about 80° C., from about 20° C. to about 80° C., from about 30° C. to about 80° C., from about 30° C. to about 70° C., preferably from about 40° C. to about 70° C., preferably from about 50° C. to about 70° C., or even more preferably from about 50° C. to about 60° C. In an embodiment, the esterase activity is measurable at a temperature from about 10° C. to about 70° C., from about 20° C. to about 80° C., from about 30° C. to about 80° C., from about 30° C. to about 70° C., preferably from about 40° C. to about 70° C., preferably from about 50° C. to about 70° C., or even more preferably from about 50° C. to about 60° C.

In an embodiment, the polypeptide having esterase activity comprises hydrolase activity or catalyse the hydrolysis of mono- terephthalic acid esters at a temperature from about 10° C. to about 80° C., from about 20° C. to about 80° C., from about 30° C. to about 80° C., from about 30° C. to about 70° C., preferably from about 40° C. to about 70° C., preferably from about 50° C. to about 70° C., or even more preferably from about 50° C. to about 60° C.

In another particular embodiment, the polypeptide having esterase activity is to catalyse the hydrolysis of di- terephthalic acid esters at a temperature from about 10° C. to about 80° C., from about 20° C. to about 80° C., from about 30° C. to about 80° C., from about 30° C. to about 70° C., preferably from about 40° C. to about 70° C., preferably from about 50° C. to about 70° C., or even more preferably from about 50° C. to about 60° C.

In an embodiment, the polypeptide described herein exhibits a measurable hydrolase/esterase activity at least in a range of pH from about 5 to about 11, preferably in a range of pH from about 6 to about 10, preferably in a range of pH from about 7 to about 10, more preferably in a range of pH from about 7.5 to about 9.5.

In an embodiment, the polypeptide described herein exhibits a measurable hydrolase/esterase activity in catalysing the hydrolysis of mono-terephthalic acid esters at least in a range of pH from about 5 to about 11, preferably in a range of pH from about 6 to about 10, preferably in a range of pH from about 7 to about 10, more preferably in a range of pH from about 7.5 to about 9.5.

In an embodiment, the polypeptide described herein exhibits a measurable hydrolase/esterase activity in catalysing the hydrolysis of di-terephthalic acid esters at least in a range of pH from about 5 to about 11, preferably in a range of pH from about 6 to about 10, preferably in a range of pH from about 7 to about 10, more preferably in a range of pH from about 7.5 to about 9.5.

In an embodiment, the polypeptide described herein exhibits a melting temperature (Tm) of from about 40° C. to about 90° C., from 50° C. to about 90° C., preferably from about 50° C. to about 80° C.

As used herein, the term “nucleic acid”, “nucleic sequence” “polynucleotide”, “oligonucleotide” and “nucleotide sequence” are used interchangeably and refer to a sequence of deoxyribonucleotides and/or ribonucleotides. The nucleic acids can be DNA (cDNA or gDNA), RNA, or a mixture of the two. It can be in single stranded form or in duplex form or a mixture of the two. It can be of recombinant, artificial and/or synthetic origin and it can comprise modified nucleotides, comprising for example a modified bond, a modified purine or pyrimidine base, or a modified sugar. The nucleic acids of the invention can be in isolated or purified form, and made, isolated and/or manipulated by techniques known per se in the art, e.g., cloning and expression of cDNA libraries, amplification, enzymatic synthesis or recombinant technology. The nucleic acids can also be synthesized in vitro by well-known chemical synthesis techniques, as described in, e.g., Belousov (1997) Nucleic Acids Res. 25:3440-3444.

The nucleic acid sequences disclosed herein may suitably be codon optimized. Suitable methods for codon optimization will be familiar to persons skilled in the art, illustrative examples of which are described in the reference manual Sambrook et al. (Sambrook et al., 2001).

The nucleic acid sequences described herein may be suitably deduced from the amino acid sequence of the polypeptides described herein and codon usage may be adapted according to the host cell in which the nucleic acid shall be transcribed.

In some embodiments, the nucleic acid sequences described herein may suitably comprise additional nucleotide sequences, such as regulatory regions, i.e., promoters, enhancers, silencers, terminators, signal peptides and the like that can be used to cause or regulate expression of the polypeptide in a selected host cell or system. Alternatively, or in addition, the nucleic acid sequences described herein may further comprise additional nucleotide sequences encoding fusion proteins, such as maltose binding protein (MBP) or glutathione S transferase (GST) that can be used to favor polypeptide expression and/or solubility.

As noted elsewhere herein, the present disclosure also extends to expression vectors and expression cassettes comprising the nucleic acid sequence described herein, optionally operably linked to one or more control sequences that direct the expression of the nucleic acid sequence in a suitable host cell. Typically, the expression vector or cassette comprises the nucleic acid sequence described herein operably linked to a control sequence such as transcriptional promoter and/or transcription terminator. The control sequence may include a promoter that is recognized by a host cell or an in vitro expression system for expression of the nucleic acid encoding the polypeptide described herein. The promoter will typically comprise a transcriptional control sequence that mediates the expression of the polypeptide. The promoter may be any polynucleotide that shows transcriptional activity in a host cell, including mutant, truncated, and hybrid promoters, and may suitably be obtained from genes encoding extracellular or intracellular polypeptides either homologous or heterologous to the host cell. The control sequence may also be a transcription terminator, which is recognized by a host cell to terminate transcription. The terminator is typically operably linked to the 3′-terminus of the nucleic acid encoding the polypeptide. Any terminator that is functional in the host cell may be used in this context. Typically, the expression vector or cassette comprises the nucleic acid sequence described herein operably linked to a transcriptional promoter and a transcription terminator.

The term “vector” typically refers to a DNA molecule used as a vehicle to transfer recombinant genetic material into a host cell. Suitable vectors include plasmids, bacteriophages, viruses, fosmids, cosmids, and artificial chromosomes. The vector is typically a DNA sequence that comprises an insert (a heterologous nucleic acid sequence, transgene) and a larger sequence that serves as the “backbone” of the vector. The purpose of a vector which transfers genetic information to the host is typically to isolate, multiply, or express the insert in the target cell. Expression vectors (also referred to as expression constructs) are specifically adapted for the expression of the heterologous sequences in the target cell, and generally have a promoter sequence that drives expression of the heterologous sequences encoding a polypeptide.

Generally, the regulatory elements that are used in an expression vector include a transcriptional promoter, a ribosome binding site, a terminator, and optionally present operator. An expression vector may further comprise an origin of replication for autonomous replication in a host cell, a selectable marker, a limited number of useful restriction enzyme sites, and a potential for high copy number. Suitable expression vectors will be familiar to persons skilled in the art, illustrative examples of which include cloning vectors, modified cloning vectors, plasmids and viruses. Expression vectors that are capable of providing suitable levels of polypeptide expression in different hosts are also well known in the art. The choice of the vector will typically depend on the compatibility of the vector with the host cell into which the vector is to be introduced.

The present disclosure also extends to a host cell comprising the nucleic acid sequence described herein. The host cell may be transformed, transfected or transduced in a transient or stable manner. The nucleic acid, expression cassette or vector is introduced into a host cell so that the nucleic acid, cassette or vector is maintained as a chromosomal integrant or as a self-replicating extra-chromosomal vector. The term “host cell” encompasses any progeny of a parent host cell that is not identical to the parent host cell due to mutations that occur during replication. The host cell may be any cell useful in the production of a variant of the present invention, e.g., a prokaryote or a eukaryote. The prokaryotic host cell may be any Gram-positive or Gram-negative bacterium. The host cell may also be a eukaryotic cell, such as a yeast, fungal, mammalian, insect or plant cell. In a particular embodiment, the host cell is selected from the group of Escherichia coli, Pseudomonas, Bacillus, Streptomyces, Trichoderma, Aspergillus, Saccharomyces, Pichia, Thermus or Yarrowia.

The nucleic acid, expression cassette or expression vector according to the invention may be introduced into the host cell by any suitable method known to persons skilled in the art, illustrative examples of which include electroporation, conjugation, transduction, competent cell transformation, protoplast transformation, protoplast fusion, biolistic “gene gun” transformation, PEG-mediated transformation, lipid-assisted transformation or transfection, chemically mediated transfection, lithium acetate-mediated transformation and liposome-mediated transformation.

In an embodiment, the host cell is a genetically modified host cell or microorganism. In this context, a host cell or microorganism may be genetically modified to enhance the expression of the polypeptide in which it is expressed and/or PETase activity of the host cell. For example, the polypeptide described herein may be used to complement a wild type strain of a fungus or bacteria known to be capable of PETase activity, in order to improve and/or increase the PETase activity of that strain.

The present disclosure also extends to a method of producing a polypeptide having esterase activity, the method comprising:
(a) providing the polynucleotide, as described herein;
(b) expressing the polynucleotide in a host cell culture, thereby producing the polypeptide; and
(c) collecting the polypeptide produced in (b) from the host cell culture.

The present invention disclosure also extends to in vitro methods of producing the polypeptide described herein, the method comprising (a) contacting a nucleic acid, cassette or vector of the invention with an in vitro expression system; and (b) recovering the polypeptide produced. In vitro expression systems are well-known by the person skilled in the art and are commercially available.

Suitable host cells will be familiar to persons skilled in the art, illustrative examples of which include a recombinant Bacillus, recombinant E. coli, recombinant Pseudomonas, recombinant Aspergillus, recombinant Trichoderma, recombinant Streptomyces, recombinant Saccharomyces, recombinant Pichia, recombinant Thermus or recombinant Yarrowia. In an embodiment, the host cell is an E. coli. In another embodiment, the host cell is a Bacillus.

The host cells may be cultivated in a nutrient medium suitable for production of polypeptides, using methods that will be known to persons skilled in the art. Suitable examples include cultivating the host cells by shake flask cultivation, or small-scale or large-scale fermentation (including continuous, batch, fed- batch, or solid state fermentations) in laboratory or industrial fermenters performed in a suitable medium and under conditions allowing the enzyme to be expressed and/or isolated. The cultivation will typically take place in a suitable nutrient medium, from commercial suppliers or prepared according to published compositions (e.g., in catalogues of the American Type Culture Collection) or any other culture medium suitable for cell growth.

Where the polypeptide is secreted into the nutrient medium, the polypeptide can be recovered directly from the culture supernatant. Conversely, the polypeptide can be recovered from cell lysates or after permeabilisation of the host cell membrane. The polypeptide may be recovered using any suitable method known to persons skilled in the art, illustrative examples of which include collection, centrifugation, filtration, extraction, spray-drying, evaporation, or precipitation. Optionally, the polypeptide may be partially or totally purified by a variety of procedures known in the art including, but not limited to, thermal shock, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction to obtain substantially pure polypeptides.

The polypeptide may be used, in purified form, either alone or in combination with additional enzymes (e.g., PETases or MHETases or carboxylesterases or cutinases having PETase activity), to catalyze enzymatic reactions involved in the degradation and/or recycling of a polyester containing material, such as plastic products containing polyester. The polypeptides described herein may be in soluble form, or on solid phase. In particular, they may be bound to cell membranes or lipid vesicles, or to synthetic supports such as glass, plastic, polymers, filter membranes, e.g., in the form of beads, columns, plates and the like.

It may be convenient to perform the method of the invention using a polypeptide that is immobilised on a substrate. Using immobilised polypeptides may be beneficial when performing the method of the invention in a semicontinuous or continuous manner.

In one embodiment, the polypeptide/s described herein is immobilised on a substrate.

The polypeptide can be immobilised on any suitable substrate using techniques known to those skilled in the art. For example, the polypeptide may be immobilised on a support resin by ion exchange, absorption (e.g. hydrophobic absorption), or covalent coupling.

In one embodiment, the polypeptide is immobilised on a resin. In one embodiment, the polypeptide is immobilised on an ion exchange resin. In one embodiment, the polypeptide is immobilised on a resin. In another embodiment, the polypeptide is immobilised on an adsorption resin. In another embodiment the polypeptide is immobilised on a nickel-affinity resin. In an embodiment the polypeptide is immobilised on a covalent resin. In one embodiment, the polypeptide is immobilised on an ion exchange resin. Those skilled in the art will be familiar with the general principle of enzymatic immobilisation technology and that principle can advantageously be applied in the context of immobilising the polypeptide on a substrate in accordance with the present invention.

Suitable ion-exchange resins for immobilising the polypeptide will generally comprise a polymer matrix or a polymer/ceramic hybrid matrix. An example of such a resin includes, but is not limited to, CM Ceramic HyperD® Ion Exchange Chromatography Resin.

In one embodiment, the ion exchange resin is a cationic exchange resin. For the operation of the method in accordance with the invention, the polypeptide will typically be immobilised on a support resin and loaded into a column.

The present disclosure also extends to compositions comprising the polypeptide, the nucleic acid or the host cell described herein.

The composition may be liquid or dry, for instance in the form of a powder. In some embodiments, the composition is a lyophilisate. For instance, the composition may comprise the polypeptide, nucleic acid and/or host cells and optionally excipients and/or reagents etc. Suitable excipients may include buffers commonly used in biochemistry, agents for adjusting pH, preservatives such as sodium benzoate, sodium sorbate or sodium ascorbate, conservatives, protective or stabilizing agents such as starch, dextrin, arabic gum, salts, sugars e.g., sorbitol, trehalose or lactose, glycerol, polyethyleneglycol, polyethene glycol, polypropylene glycol, propylene glycol, divalent ions such as calcium, sequestering agent such as EDTA, reducing agents (e.g., beta-mercaptoethanol, dithiothreitol, ascorbic acid, tris(2-carboxyethyl)phosphine), amino acids, a carrier such as a solvent or an aqueous solution, and the like.

In an embodiment, the composition comprises the polypeptide described herein (the polypeptide may be present in the composition in an isolated or at least partially purified form). In an embodiment, the composition comprises the polypeptide described herein in an amount of from about 0.1% to about 99.9%, preferably from about 0.1% to about 50%, preferably from about 0.1% to about 30%, preferably from about 0.1% to about 5% by weight of the total weight of the composition. In a preferred embodiment, the composition comprises the polypeptide described herein in an amount of from about 0.1 to about 5% by weight of the total weight of the composition. In another embodiment, the composition comprises the polypeptide described herein in an amount of from about 0.1 to about 0.2% by weight of the total weight of the composition. The amount of polypeptide in the composition may suitably adapted by persons skilled in the art, depending e.g., on the nature and/or amount of the polyester containing material to be degraded (hydrolysed) and/or the presence or absence of any additional enzymes/polypeptides in the composition.

The compositions described herein may further comprise additional polypeptide(s) exhibiting enzymatic activity, not limited to PETase, esterase, carboxylesterases, MHETase or cutinase with promiscuous PETase/MHETase activities.

In an embodiment, the polypeptide described herein is solubilized in an aqueous medium together with one or more excipients, such as excipients that may suitably stabilize or protect the polypeptide from degradation. For example, the polypeptides described herein may be solubilized in water and then admixed with excipients such as glycerol, sorbitol, dextrin, starch, glycol such as propanediol, salt, etc. The resulting admixture may then be dried so as to obtain a powder. Methods for drying such mixture are well known to the one skilled in the art and include, without limitation, lyophilisation, freeze-drying, spray-drying, supercritical drying, down-draught evaporation, thin-layer evaporation, centrifugal evaporation, conveyor drying, fluidized bed drying, drum drying or any combination thereof.

In an embodiment, the composition comprises at least one host cell expressing the polypeptide described herein, or an extract thereof. By “extract of a cell” is meant any fraction obtained from a cell, such as cell supernatant, cell debris, cell walls, DNA extract, enzymes or enzyme preparation or any preparation derived from cells by chemical, physical and/or enzymatic treatment, which is essentially free of living cells. Preferred extracts are enzymatically-active extracts. The composition may comprise one or several host cells or extract thereof containing the polypeptide described herein, and optionally one or several additional cells.

As noted elsewhere herein, the present inventors have surprisingly found that the polypeptides described herein (the engineered polypeptide and its variants) have greater esterase activity, that is the hydrolysis of mono- and di- terephthalic acid esters when compared to the extant PETases and cutinases with PETase activity. Thus, disclosed herein is a method of hydrolysing mono- and di-terephthalic acid esters, the method comprising exposing the terephthalic acid esters to the polypeptide, the composition or the host cell described herein, under conditions sufficient to enable the polypeptide to convert the monoester terephthalate to terephthalic acid and an alcohol; diester terephthalate to a monoester terephthalate and an alcohol; or diester terephthalate to terephthalic acid and an alcohol. The present disclosure also extends to a method of degrading a plastic product comprising a polyester, the method comprising exposing the plastic product to the polypeptide, the composition or the host cell described herein.

The present disclosure extends to the use of the polypeptide, the composition or the host cell described herein in a process for degrading a polyester in aerobic or anaerobic conditions and/or recycling polyester containing material, as plastic products made of or containing polyesters and/or producing biodegradable plastic products containing polyester. Such methods and uses are particularly useful for degrading a plastic product comprising PET.

Advantageously, the polyester(s) of the polyester containing material is (are) depolymerized up to monomers and/or oligomers. In an embodiment, at least one polyester is degraded to yield re-polymerizable monomers and/or oligomers, which are advantageously retrieved or recovered for further use.

As noted elsewhere herein, the plastic product may comprise at least one polyester selected from the group consisting of polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA) and combinations of any of the foregoing. The plastic product may comprise at least one polymer selected from the group consisting of polypropylene, polystyrene, polyvinyl chloride, synthetic rubber, phenol formaldehyde resin (or Bakelite), neoprene, nylon, polyacrylonitrile, PVB, and silicone.

The time required for degrading a polyester containing material may vary depending on the polyester containing material itself (i.e., nature and origin of the plastic product, its composition, shape etc.), the type and amount of polypeptide used, as well as various process parameters (i.e., temperature, pH, additional agents, etc.). One skilled in the art may easily adapt the process parameters to the polyester containing material.

Advantageously, the degrading process is implemented at a temperature from about 10° C. to about 80° C., preferably from about 20° C. to about 80° C., preferably from about 30° C. to about 80° C., preferably from about 40° C. to about 80° C., preferably from about 50° C. to about 80° C., even preferably from about 60° C. to about 80° C., even more preferably at about 60° C. to about 70° C., even more preferably at about 60° C. As the skilled person will appreciate, the temperature is typically maintained at an activating temperature, which corresponds to the temperature at which the polypeptide is active and/or the recombinant microorganism does synthesize, produce or release the polypeptide described herein. In an embodiment, the temperature is maintained below the glass transition temperature (Tg) of the polyester in the polyester containing material. In an embodiment, the degrading process or method is implemented at a temperature from about 10° C. to about 80° C., preferably from about 20° C. to about 80° C., preferably from about 30° C. to about 80° C., preferably from about 40° C. to about 80° C., preferably from about 50° C. to about 80° C., even preferably from about 60° C. to about 80° C., even more preferably at about 60° C. to about 70° C., even more preferably at about 60° C. The process or method may suitably be implemented in a continuous way, at a temperature at which the polypeptide can be used several times and/or recycled.

Advantageously, the degrading process or method is implemented at a pH in a range from about 5 to about 11, preferably in a range from about 6 to about 10, preferably at a pH from about 6.5 to about 9, preferably in a range from about 7 to about 9, preferably in a range from about 7 to about 8, preferably at a pH from about 9.5 to about 11.

In an embodiment, the polyester-containing material may be pre-treated prior to being contacted with the polypeptide in order to physically or chemically change its structure, so as to improve access of the polyester and its intermediates with the enzyme/s.

Monomers resulting from the depolymerization or degradation process or method may be suitably recovered, sequentially or continuously. A single type of monomers or several different types of monomers may be recovered, depending on the starting polyester containing material.

The recovered monomers may be further purified, using any suitable purifying method and conditioned in a repolymerizable form. Illustrative examples of suitable purifying methods include stripping process, separation by aqueous solution, steam selective condensation, filtration and concentration of the medium after the bioprocess, separation, distillation, vacuum evaporation, extraction, electrodialysis, adsorption, ion exchange, precipitation, crystallization, concentration and acid addition dehydration and precipitation, nanofiltration, acid catalyst treatment, semi continuous mode distillation or continuous mode distillation, solvent extraction, evaporative concentration, evaporative crystallization, liquid/liquid extraction, hydrogenation, azeotropic distillation process, adsorption, column chromatography, simple vacuum distillation and microfiltration, combined or not.

The repolymerizable monomers may be used to synthesize new polyesters. Advantageously, polyesters of the same nature are repolymerized. However, it is possible to mix the recovered monomers with other monomers, for example, in order to synthesize new copolymers. Alternatively, the recovered monomers may be used as chemical intermediates in order to produce new chemical compounds of interest.

The present disclosure also extends to a composition comprising a plastic compound and the polypeptide, and/or host cell expressing said polypeptide or an extract thereof containing said polypeptide.

The present disclosure also extends to a masterbatch composition comprising the polypeptide, composition and/or host cell expressing said polypeptide or an extract thereof containing said polypeptide.

Advantageously, such plastic compound or masterbatch composition described herein can be used for the production of a polyester containing material.

In an embodiment, the resulting plastic compound or masterbatch composition is a biodegradable plastic compound or masterbatch composition complying with at least one of the relevant standards and/or labels known by the person skilled in the art, such as standard EN 13432, standard ASTM D6400, OK Biodegradation Soil (Label Vincotte), OK Biodegradation Water (Label Vincotte), OK Compost (Label Vincotte), OK Home Compost (Label Vincotte).

Advantageously, the degrading process of the polyester containing material (i.e., plastic compound or masterbatch composition or plastic product) is implemented at a temperature from about 10° C. to about 80° C., preferably from about 20° C. to about 80° C., preferably from about 30° C. to about 80° C., preferably from about 40° C. to about 80° C., preferably from about 50° C. to about 80° C., even preferably from about 60° C. to about 80° C., even more preferably from about 60° C. to about 70° C., even more preferably at about 60° C.+/−5° C.

Alternatively, the degrading process of the polyester containing material (i.e., plastic compound, masterbatch composition or plastic product) is implemented at a temperature from about 50° C. to about 70° C., more preferably at 60° C., +/−5° C.

The engineered polypeptides having esterase activity disclosed herein are suitable for a range of application, including industrial applications, illustrative examples of which include as additives in detergents, feed compositions (including for animal feed), textiles production, electronics and biomedical applications. For example, the engineered polypeptide having esterase activity disclosed herein can be employed in textile processing or textile production, where it can be used as an exoesterase to suitably modify the properties of textile fibres.

The invention will now be described with reference to the following Examples which illustrate some preferred aspects of the present invention. However, it is to be understood that the particularity of the following description of the invention is not to supersede the generality of the preceding description of the invention.

### EXAMPLES

### Example 1: Ancestral Sequence Reconstruction

All amino acid sequences belonging to the protein families PF12695, PF01738 and PF12740 were retrieved from pfam. Redundancy was removed to 100% sequence identity using CD-HIT (Fu et al. 2012) and an all-vs-all pBLAST was performed. The resulting table was visualised as a sequence similarity network in cytoscape (Shannon et al. 2003) and edges connecting sequences with less than 40% sequence identity were deleted before the network was visualised as a prefuse-force directed network graph. All sequences belonging to the sequence cluster that included LCC, TfCut2 and IsPETase were retrieved fromuniprot and redundancy was removed to 95% sequence identity using CD-HIT. Signal peptides for Gram-positive and negative bacteria were identified with SignalP5.0 (Almagro Armenteros et al. 2019) and manually removed. A multiple sequence alignment of this dataset was produced using the GINSI protocol in MAFFT (Katoh and Standley 2013), which was manually edited according to solved crystal structures. The final alignment had 397 aligned sequences.

100 independent replicates of maximum likelihood (ML) tree search and phylogenetic reconstruction were performed in IQ-TREE (Minh et al. 2020) using default tree search parameters. The sequence evolution model of best-fit (WAG+F+R8) (Whelan and Goldman 2001) was identified by Akaike and Bayesian information criteria in ModelFinder (Kalyaanamoorthy et al. 2017). Branch supports for each inference were calculated as alternate likelihood ratio test statistics (Anisimova and Gascuel 2006) and ultrafast bootstrap approximation (ufboot) (Hoang et al. 2018) to 1000 replicates. Because the approximately unbiased (AU) (Shimodaira 2002) test conducted to 10000 replicates failed to reject any single topology as statistically less likely than any other, 20 converged topologies that represented most of the topological diversity within the full dataset were sampled for ASR. Ancestral sequences were reconstructed over the 20 topologies by ML in CodeML from the PAML (Yang 2007) suite using the sequence evolution model WAG+G4. 18 conserved insertions that had been identified in the extant sequences were treated as discrete binomial traits (1 being the insertion is present, 0 being the insertion is absent) and were reconstructed by ML in the R package Ape (Paradis, Claude, and Strimmer 2004) using a binary Jukes-Cantor-like equal rates model. 48 reconstructed ancestral sequences were sampled from common nodes shared between LCC and TfCut2 over the 20 topologies that had a minimum ufboot support of 0.9 and a minimum mean posterior probability of 0.8.

### Example 2: Protein Expression and Purification

Plasmids were transformed by heat shock into chemically competent E. cloni@cells (Lucigen) and plated onto Lysogeny broth (LB) agar supplemented with 100 μg/L kanamycin and incubated at 37° C. overnight. A single colony was used to inoculate 1.5 mL autoinduction media supplemented with 100 μg/mL kanamycin in a 2.2 mL 96-well deep well block and grown at 1050 rpm at 37° C. for 5 hours, followed by room temperature (RT; 25° C.) for 16 hours.

Cells were harvested by centrifugation at 2000×g for 15 minutes at RT and resuspended in Lysis Buffer (1× BugBuster® Protein Extraction Reagent (Merck-Millipore), 20 mM Tris, 300 mM NaCl, 1 U/ml Turbonuclease (Sigma) pH 8). The cell suspension was left to incubate at RT for 20 minutes with gentle shaking. The lysate was separated from the insoluble cell debris by centrifugation at 2250×g for 1 hour at RT. The clarified lysate was then diluted with 100 μl of Equilibration Buffer (20 mM Tris, 300 mM NaCl pH 8) and purified by nickel-charged IMAC using a 96-well HisPur™ Ni-NTA Spin Plate (ThermoFisher Scientific) equilibrated in Equilibration Buffer, washing the sample three times with 250 μl of Wash Buffer (20 mM Tris, 300 mM NaCl, 10 mM imidazole pH 8) and eluting with 250 μl of Elution Buffer (20 mM Tris, 300 mM NaCl, 150 mM imidazole pH 8). All centrifugation steps following addition of Wash or Elution Buffer were at 1000×g for 1 minute at RT. The eluate was stored at 4° C.

### Example 3: HPLC Activity Assay to Detect Hydrolysis of MOCT and DOCT

Assays of enzymatic activity against monooctyl terephthalate (MOCT) or dioctyl terephthalate (DOCT) were conducted with 1.5 mM substrate, 5% DMSO, and a 1:10 dilution of the eluate from the Ni-NTA purification in reaction buffer (45 mM NaH₂PO₄, 90 mM NaCl, pH 7.5).

Reactions were incubated at 50° C. for 64 minutes then quenched by heating at 95° C. for at least 10 minutes. The reactions were analysed using high-performance liquid chromatography (HPLC) and compared to control reactions containing no enzyme.

Concentrations of terephthalic acid and monooctyl terephthalate were determined by comparison to calibration curves generated using synthesised or commercial standards.

For comparison of activity of all variants, the absorbance at 254 nm was used. The retention time and corresponding calibration curve of TPA for the HPLC method was obtained using TPA standard (Sigma Aldrich). To characterise the MOCT and DOCT hydrolysis activity of the selected variants, the peak corresponding to TPA, based on comparison to the standard sample, was integrated to obtain peak area. Selected variants showing MOCT and DOCT hydrolysis activity are shown in Table 1.

The polypeptides having SEQ ID NOs: 32 and 34 appear to have higher activity against DOCT than MOCT, while SEQ ID NOs: 44-54 appear to have significantly higher activity against MOCT than DOCT. SEQ ID NOs: 6-8 demonstrated efficient hydrolysis of DOCT while SEQ ID NOs: 9-11 demonstrated efficient hydrolysis of DOCT (see Tables 2-3).

Analyses of SEQ ID NOs: 1-118 demonstrating activity in hydrolysing MOCT and DOCT indicated that they each fall within a consensus sequence—SEQ ID NO: 119.

TABLE 1
Amino acid sequences of exemplary MOCTase and DOCTase enzymes

| SEQ ID | |
|---|---|
| NO | Sequences |
| 1 | MAANPYERGPDPTEASLEASSGPFSVSETSVSRLSASGFGGGTIYYPTTTSSGTYGAVAISP |
| | GYTATQSSIAWLGPRLASHGFVVITIDTNTTFDQPDSRARQLMAALNYLVNRSSVRSRIDS |
| | SRLAVMGHSMGGGGTLRAAEDNPSLKAAIPLTPWHTNKNWSSVRVPTLIIGAENDTIAPV |
| | SSHAKPFYNSLPSSTPKAYLELNGASHFAPNSSNTTIGKYSVSWLKRFVDNDTRYSQFLCP |
| | APHDDSAISEYRSTCPY |
| 2 | MAENPYERGPDPTESSIEALRGPFAVSEESVSRLSVSGFGGGTIYYPTDTSEGTFGAVAISP |
| | GYTGTQSSMAWLGPRIASQGFVVFTIDTNTTYDQPDSRARQLQAALDYLVEDSSVRDRID |
| | PNRLGVMGHSMGGGGTLRAAEDRPDLKAAIPLTPWHLNKNWSSVRVPTLIIGAENDTIAP |
| | VRTHAEPFYESLPSSLDKAYLELDGASHFAPNISNTTIAKYSISWLKRFVDDDTRYEQFLCPP |
| | PRTGSEISEYRSTCPF |
| 3 | MAENPYERGPDPTEASIEAERGPFAIAQVSVPAGSGSGFGGGTIYYPTDTSQGTFGAVAISP |
| | GFTATEASIAWLGPRLASQGFVVITIDTNSRYDQPSARADQLLAALDYLTQSSSVRSRIDPN |
| | RLAVMGHSMGGGGTLEAAENRPSLKAAIPLAPWNTNKNWSSVRVPTMIIGAQNDTIAPVG |
| | SHAEPFYNSLPASPEKAYLELNGADHFAPTSSNTTIAKYSISWLKRFVDDDTRYDQFLCPAP |
| | SPDSAISEYRSTCPH |
| 4 | MQANPYQRGPDPTSASLEASSGPFSVSTTSVSRLSASGFGGGTIYYPTTTSSGKYGAVAIS |
| | PGYTATQSSIAWLGRRLASHGFVVITIDTNTTLDQPDSRATQLMAALNYVVNSSTVRSRVD |
| | ASRLAVMGHSMGGGGTLIAAENNPSLKAAIPLTPWHTSKNFSSVRVPTLIIGAENDTIAPVS |
| | SHAKPFYNSLPSTTSKAYLELNGASHFAPNSSNTPIGKYSISWMKRFVDNDTRYSPFLCGA |
| | PHQGAVISEYRDNCPY |
| 5 | MAANPYERGPNPTEALLEARSGPFSVSEERASRLGADGFGGGTIYYPRENSDNTYGAVAIS |
| | PGYTGTQASVAWLGERIASHGFVVITIDTNTTLDQPDSRARQLNAALDYMINDSAVRSRID |
| | SSRLAVMGHSMGGGGTLRLASQRPDLKAAIPLTPWHLNKNWSSVRVPTLIIGADLDTIAPV |
| | LTHAEPFYNSLPTSISKAYLELDGATHFAPNITNKTIGKYSVAWLKRFVDEDTRYTQFLCPGP |
| | RTGSDVEEYRSTCPF |
| 6 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELDGATHFAPNIPNKIIGKYSVAWLKRFVDNDTRYTQFLCPGP |
| | RDGGEVEEYRSTCPF |
| 7 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALNYMINRSTVRSRIDS |
| | SRLAVMGHSMGGGGTLRLASQRPDLKAAIPLTPWHLNKNWSSVTVPTLIIGADLDTIAPVA |
| | THAKPFYNSLPSSISKAYLELDGATHFAPNIPNKIIGKYSVAWLKRFVDNDTRYTQFLCPGP |
| | RDGGEVEEYRSTCPF |
| 8 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALNYMINRSTVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELDGATHFAPNIPNKIIGKYSVAWLKRFVDNDTRYTQFLCPGP |
| | RDGGEVEEYRSTCPF |
| 9 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRAEQLNAALNYMINRSTVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELDGATHFAPNIPNKIIGKYSVAWLKRFVDNDTRYTQFLCPGP |
| | RDGGEVEEYRSTCPF |
| 10 | MAANPYERGPNPTDALLEARSGPFSVSEENVSRLSASGFGGGTIYYPRENSSNTYGAVAIS |
| | PGYTGTEASIAWLGERIASHGFVVITIDTITTLDQPDSRAEQLNAALNHMINRSTVRSRIDS |
| | SRLAVMGHSMGGGGTLRLASQRPDLKAAIPLTPWHLNKNWSSVTVPTLIIGADLDTIAPVA |
| | THAKPFYNSLPSSISKAYLELDGATHFAPNIPNKIIGKYSVAWLKRFVDNDTRYTQFLCPGP |
| | RDGGEVEEYRSTCPF |
| 11 | MAANPYERGPNPTDALLEARSGPFSVSEENVSRLSASGFGGGTIYYPRENSENTYGAVAISP |
| | GYTGTEASIAWLGERIASHGFVVITIDTITTLDQPDSRAEQLNAALNHMINRSTVRSRIDSS |
| | RLAVMGHSMGGGGTLRLASQRPDLKAAIPLTPWHLNKNWSSVTVPTLIIGADLDTIAPVAT |
| | HAKPFYNSLPSSISKAYLELDGATHFAPNIPNKIIGKYSVAWLKRFVDNDTRYTQFLCPGPR |
| | DGGEVEEYRSTCPF |
| 12 | MAANPYERGPDPTESSLEASSGPFSVSETSVSRLSASGFGGGTIYYPTTTSEGTYGAVAISP |
| | GYTATQSSIAWLGPRLASHGFVVITIDTNTTFDQPDSRARQLMAALNYLVNRSSVRSRIDS |
| | SRLAVMGHSMGGGGTLRAAEDNPSLKAAIPLTPWHTNKNWSSVRVPTLIIGAENDTIAPV |
| | SSHAKPFYNSLPSSTPKAYLELNGASHFAPNSSNTTIGKYSIAWLKRFVDNDTRYSQFLCPA |
| | PHDDSAISEYRSTCPY |
| 13 | MAANPYERGPDPTESSLEASSGPFSVSQTSVSRLSVSGFGGGTIYYPTDTSSGTYGAVAIS |
| | PGYTATQSSIAWLGPRLASHGFVVITIDTNSTFDQPDSRARQLMAALDYLVNRSPVRSRID |
| | SSRLAVMGHSMGGGGTLRAAEDNPSLKAAIPLTPWHTDKNWSSVRVPTLIIGAENDTIAP |
| | VSSHAKPFYNSLPSSTSKAYLELNGASHFAPNSSNTTIGKYSVSWLKRFVDNDTRYSQFLC |
| | PAPHDDSAISEYRSTCPY |
| 14 | MADNPYERGPAPTESSIEASRGPFAVSQTSVSSLSVSGFGGGTIYYPTSTSEGTFGAVAISP |
| | GYTATQSSIAWLGPRLASQGFVVFTIDTNTRYDQPDSRGRQLLAALDYLTQRSSVRSRIDA |
| | SRLGVMGHSMGGGGTLEAAEDRPSLQAAIPLTPWNLDKNWSSVRVPTMIIGAENDTIAPV |
| | SSHSEPFYTSLPSSLDKAYLELNGASHFAPNTSNTTIAKYSISWLKRFIDNDTRYEQFLCPAP |
| | SRDTTISEYRDTCPH |
| 15 | MADNPYERGPDPTESSIEASRGPFAVSQTSVSRLSVSGFGGGTIYYPTSTSEGTFGAVAISP |
| | GYTATQSSIAWLGPRLASQGFVVFTIDTNTRYDQPDSRGDQLLAALDYLTQRSSVRSRIDP |
| | SRLAVMGHSMGGGGTLEAAKDRPSLKAAIPLTPWNTDKNWSSVTVPTLIIGAENDTIAPVS |
| | SHSKPFYNSLPSSPEKAYLELNGASHFAPNSSNTTIAKYSIAWLKRFVDNDTRYSQFLCPAP |
| | SADSAISEYRDTCPY |
| 16 | MQANPYQRGPDPTSSSLEASSGPFSVSTTSVSRLSVSGFGGGTIYYPTNTSSGTYGAVAIS |
| | PGYTATQSSIAWLGPRLASHGFVVITIDTNSTFDQPDSRATQLMAALDYVVNSSPVRSRVD |
| | SSRLAVMGHSMGGGGTLRAAEDNPSLKAAIPLTPWHTDKNFSSVRVPTLIIGAENDTVAPV |
| | SSHAKPFYNSLPSSTPKAYLELNGASHFAPNSSNTTIGKYSVSWMKRFVDNDTRYSQFLCP |
| | APHDDSAISEYRSTCPY |
| 17 | MQANPYQRGPDPTESSLEASSGPFSVSTTSVSRLSVSGFGGGTIYYPTSTSSGTYGAVAIS |
| | PGYTATQSSIAWLGPRLASHGFVVITIDTNSTFDQPDSRATQLMAALNYLVNSSSVRSRID |
| | SSRLAVMGHSMGGGGTLRAAEDNPSLKAAIPLTPWHTDKNFSSVTVPTLIIGAENDTIAPV |
| | SSHAKPFYNSLPSSTSKAYVELNGASHFAPNSSNTTIGKYSVSWMKRFVDNDTRYSQFLC |
| | GAPHDDSAISEYRSNCPY |
| 18 | MQANPYQRGPDPTSASLEASSGPFSVSTTSVSRLSASGFGGGTIYYPTTTSSGTYGAVAIS |
| | PGYTATQSSIAWLGPRLASHGFVVITIDTNSTFDQPDSRATQLMAALNYVVNSSTVRSRVD |
| | SSRLAVMGHSMGGGGTLQAAEDNPSLKAAIPLTPWHTNKNFSSVRVPTLIIGAENDTIAPV |
| | SSHAKPFYNSLPSSTSKAYVELNGASHFAPNSSNTTIGKYSVSWMKRFVDNDTRYSPFLCG |
| | APHDDSAISEYRSTCPY |
| 19 | MAANPYERGPDPTNASLEASSGPFSVSETSVSRLSASGFGGGTIYYPTSTSEGTYGAVAISP |
| | GYTGTQSSIAWLGPRLASHGFVVITIDTNTTLDQPDSRASQLMAALNYLVNRSTVRSRIDA |
| | SRLAVMGHSMGGGGTLRAAEQRPSLKAAIPLTPWHTDKNWSSVRVPTLIIGAENDTIAPVS |
| | SHAKPFYNSLPSTISKAYLELNGASHFAPNSSNTTIGKYSISWLKRFVDNDTRYSQFLCPAP |
| | RQGALIEEYRDTCPY |
| 20 | MQANPYQRGPDPTSSSLEASSGPFSVSTTSVSSLSVSGFGGGTIYYPTNTSSGTYGAVAIS |
| | PGYTATQSSIAWLGPRLASHGFVVITIDTNSTFDQPDSRARQLMAALNYLVNRSPVRSRVD |
| | SSRLAVMGHSMGGGGTLRAAEDNPSLKAAIPLTPWHTDKNFSSVRVPTLIIGAENDTIAPV |
| | SSHAKPFYNSLPSSTSKAYLELNGASHFAPNSSNTTIGKYSVSWMKRFVDNDTRYSQFLCP |
| | APHDDSAISEYRDTCPY |
| 21 | MAANPYERGPDPTESSLEASSGPFSVSTTSVSRLSVSGFGGGTIYYPTSTSEGTYGAVAISP |
| | GYTATQSSIAWLGRRLASHGFVVITIDTNTTFDQPDSRADQLMAALNYLVNRSSVRSRIDS |
| | SRLAVMGHSMGGGGTLRAAKDRPSLKAAIPLTPWHTDKNWSSVTVPTLIIGAENDTIAPVS |
| | SHAKPFYNSLPSSTSKAYLELNGASHFAPNSSNTTIGKYSVAWLKRFVDNDTRYSQFLCPA |
| | PHDGSAISEYRDTCPY |
| 22 | MQANPYQRGPDPTSASLEASSGPFSVSTTSVSRLSASGFGGGTIYYPTSTSSGTYGAVAIS |
| | PGYTATQSSIAWLGRRLASHGFVVITIDTNTTLDQPDSRATQLMAALNYLVNRSTVRSRID |
| | ASRLAVMGHSMGGGGTLRAAEQNPSLKAAIPLTPWHTDKNFSSVRVPTLIIGAENDTIAPV |
| | SSHAKPFYNSLPSTTSKAYLELNGASHFAPNSSNTPIGKYSISWMKRFVDNDTRYSQFLCG |
| | APHQGAVISEYRDNCPY |
| 23 | MAENPYERGPDPTESSIEATRGPFAVSQTSVSRLSVSGFGGGTIYYPTSTSEGTFGAVAISP |
| | GYTATQSSIAWLGPRLASQGFVVITIDTNSRYDQPASRGDQLLAALDYLTQSSSVRSRIDP |
| | SRLAVMGHSMGGGGTLEAAKDRPSLKAAIPLTPWNTDKNWSEVRVPTLIIGAENDTVAPV |
| | SSHAEPFYNSLPSSPEKAYLELNGASHFAPNSSNTTIAKYSISWLKRFVDDDTRYDQFLCPA |
| | PSPDSAISEYRSTCPH |
| 24 | MAANPYERGPDPTEASLEASSGPFSVSQTSVSSLSVSGFGGGTIYYPTSTSSGTYGAVAISP |
| | GYTATQSSIAWLGPRLASHGFVVITIDTNSTFDQPDSRARQLMAALDYLVNRSSVRSRIDS |
| | SRLAVMGHSMGGGGTLRAAEDRPSLKAAIPLTPWHTNKNWSSVRVPTLIIGAENDTIAPVS |
| | SHAKPFYNSLPSSTSKAYLELNGASHFAPNSSNTTIGKYSVSWLKRFVDNDTRYSQFLCPA |
| | PHDDSAISEYRDTCPY |
| 25 | MAANPYERGPDPTEASLEASSGPFSVSETSVSRLSASGFGGGTIYYPTTTSEGTYGAVAISP |
| | GYTATQSSIAWLGPRLASHGFVVITIDTNSTFDQPDSRADQLMAALNYLVNRSSVRSRIDS |
| | SRLAVMGHSMGGGGTLQAAEDRPSLKAAIPLTPWHTNKNWSSVRVPTLIIGAENDTIAPV |
| | SSHAKPFYNSLPSSTSKAYLELNGASHFAPNSSNTTIGKYSVSWLKRFVDNDTRYSQFLCP |
| | APHDDSAISEYRSTCPY |
| 26 | MQANPYQRGPDPTSSSLEASSGPFSVSTTSVSRLSASGFGGGTIYYPTTTSSGTYGAVAIS |
| | PGYTATQSSIAWLGRRLASHGFVVITIDTNSTFDQPDSRATQLMAALNYLVNSSTVRSRID |
| | ASRLAVMGHSMGGGGTLRAAEDNPSLKAAIPLTPWHTDKNFSSVRVPTLIIGAENDTIAPV |
| | STHAKPFYNSLPSSTSKAYVELNGASHFAPNSSNTPIGKYSISWMKRFVDNDTRYSQFLCG |
| | APHDGSAISEYRDNCPY |
| 27 | MADNPYERGPAPTEASIEASRGPFAISQVSVPSASGSGFGGGTIYYPTDTSQGTFGAVAISP |
| | GFTATEASIAWLGPRLASQGFVVITIDTNSRYDQPDSRGKQLLAALDYLTQKSSVRSRIDP |
| | NRLAVMGHSMGGGGTLEAAENRPSLKAAIPLTPWHTDKNWSSVRVPTMIIGAENDTIAPV |
| | GSHAEPFYNSLPSSPEKAYLELKGADHFAPTSSNTTIAKYSISWLKRFVDDDTRYDQFLCPA |
| | PSSDSAISEYRDTCPH |
| 28 | MAENPYERGPAPTESSIEATRGPFAVSQTSVSSLSVSGFGGGTIYYPTSTSEGTFGAVAISP |
| | GYTASQSSIAWLGPRLASQGFVVFTIDTNTRYDQPASRGDQLLAALDYLTQRSSVRSRIDA |
| | SRLGVMGHSMGGGGTLEAAKDRPSLQAAIPLTGWNLDKNWSEVRVPTLVIGAENDTIAPV |
| | SSHSEPFYNSLPSSLDKAYLELNGASHFAPNSSNTTIAKYSISWLKRFIDNDTRYEQFLCPA |
| | PRPGSTIEEYRDTCPH |
| 29 | MAANPYERGPDPTESSLEASSGPFSVSQTSVSRLSVSGFGGGTIYYPTSTSSGTYGAVAISP |
| | GYTATQSSIAWLGPRLASHGFVVITIDTNSTFDQPDSRADQLMAALNYLVNRSSVRSRIDS |
| | SRLAVMGHSMGGGGTLRAAEDRPSLKAAIPLTPWHTDKNWSSVTVPTLIIGAENDTIAPVS |
| | SHAKPFYNSLPSSTSKAYLELNGASHFAPNSSNTTIGKYSISWLKRFVDNDTRYSQFLCPAP |
| | HDDSAISEYRSTCPY |
| 30 | MAANPYERGPDPTESSLEASSGPFSVSETSVSRLSVSGFGGGTIYYPTSTSSGTYGAVAISP |
| | GYTATQSSIAWLGPRLASHGFVVITIDTNSTFDQPDSRARQLMAALDYLVNSSSVRSRIDS |
| | SRLAVMGHSMGGGGTLRAAEDRPSLKAAIPLTPWHTNKNWSSVRVPTLIIGAENDTVAPV |
| | SSHAKPFYNSLPSSTPKAYLELNGASHFAPNSSNTTIGKYSVAWLKRFVDNDTRYSQFLCP |
| | APHDDSAISEYRSTCPY |
| 31 | MAANPYERGPDPTESSLEASSGPFSVSETSVSRLSVSGFGGGTIYYPTDTSEGTYGAVAISP |
| | GYTATQSSIAWLGPRLASHGFVVITIDTNSTFDQPDSRARQLQAALNYLVNSSSVRSRIDS |
| | NRLAVMGHSMGGGGTLRAAEDNPSLKAAIPLTPWHTNKNWSSVTVPTLIIGAENDTIAPVS |
| | SHAKPFYNSLPSSTSKAYLELNGASHFAPNSSNTTIGKYSISWLKRFVDNDTRYSQFLCPAP |
| | HDDSAISEYRSTCPY |
| 32 | MQANPYQRGPDPTSSSLEASSGPFSVSTTSVSRLSVSGFGGGTIYYPTTTSSGTYGAVAIS |
| | PGYTATQSSIAWLGPRLASHGFVVITIDTNSTFDQPDSRATQLMAALNYVVNSSPVRSRVD |
| | SSRLAVMGHSMGGGGTLRAAEDNPSLKAAIPLTPWHTNKNFSSVRVPTLIIGAENDTVAPV |
| | SSHAKPFYNSLPSSTPKAYLELNGASHFAPNSSNTTIGKYSVAWMKRFVDNDTRYSPFLCG |
| | APHDDSAISEYRSTCPY |
| 33 | MAANPYERGPDPTEASLEASSGPFSVSTTSVSSLSVSGFGGGTIYYPTSTSEGTYGAVAISP |
| | GYTATQSSIAWLGRRLASHGFVVITIDTNSTFDQPDSRADQLMAALNYLVNRSSVRSRIDS |
| | SRLAVMGHSMGGGGTLRAAKDRPSLKAAIPLTPWHTDKNWSSVRVPTLIIGAENDTIAPV |
| | SSHAKPFYNSLPSSTSKAYLELNGASHFAPNSSNTTIGKYSVSWLKRFVDNDTRYSQFLCP |
| | APHDDSAISEYRDTCPY |
| 34 | MAENPYERGPAPTESSIEASRGPFAVSQTSVSSLSVSGFGGGTIYYPTSTSEGTFGAVAISP |
| | GYTATQSSIAWLGPRLASQGFVVFTIDTNTRYDQPASRGDQLLAALDYLTQRSSVRSRIDA |
| | SRLGVMGHSMGGGGTLEAAKDRPSLQAAIPLTPWNLDKNWSEVRVPTLIIGAENDTIAPV |
| | SSHSEPFYNSLPSSLDKAYLELNGASHFAPNSSNTTIAKYSISWLKRFIDNDTRYEQFLCPA |
| | PSPDSTISEYRDTCPH |
| 35 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRAEQLNAALNYMINRSTVRSRIDS |
| | SRLAVMGHSMGGGGTLRLASQRPDLKAAIPLTPWHLNKNWSSVTVPTLIIGADLDTIAPVA |
| | THAKPFYNSLPSSISKAYLELDGATHFAPNIPNKIIGKYSVAWLKRFVDNDTRYTQFLCPGP |
| | RDGGEVEEYRSTCPF |
| 36 | MAANPYERGPDPTESSLEASSGPFSVSETSVSRLSVSGFGGGTIYYPTSTSSGTYGAVAISP |
| | GYTATQSSIAWLGPRLASHGFVVITIDTNTTFDQPDSRARQLMAALNYLVNSSSVRSRIDS |
| | SRLAVMGHSMGGGGTLRAAEDNPSLKAAIPLTPWHTDKNWSSVRVPTLIIGAENDTIAPV |
| | SSHAKPFYNSLPSSTPKAYLELNGASHFAPNSSNTTIGKYSISWLKRFVDNDTRYSQFLCPA |
| | PHDDSAISEYRSTCPY |
| 37 | MAANPYERGPDPTEASLEATSGPFSVSETSVSRLSVSGFGGGTIYYPTDTSSGTYGAVAISP |
| | GYTATQSSIAWLGPRLASHGFVVITIDTNSTFDQPDSRARQLMAALNYLVNSSPVRSRIDS |
| | SRLAVMGHSMGGGGTLRAAEDNPSLKAAIPLTPWHTNKNWSSVRVPTLIIGAENDTIAPV |
| | SSHAKPFYNSLPSSTSKAYLELNGASHFAPNSSNTTIGKYSISWLKRFVDNDTRYSQFLCPA |
| | PHDGSAISEYRSTCPY |
| 38 | MAANPYERGPNPTDALLEARSGPFSVSEENVSRLSASGFGGGTIYYPRENSDNTYGAVAIS |
| | PGYTGTEASIAWLGERIASHGFVVITIDTITTLDQPDSRAEQLNAALNHMINRSTVRSRIDS |
| | SRLAVMGHSMGGGGTLRLASQRPDLKAAIPLTPWHLNKNWSSVTVPTLIIGADLDTIAPVA |
| | THAKPFYNSLPSSISKAYLELDGATHFAPNIPNKIIGKYSVAWLKRFVDNDTRYTQFLCPGP |
| | RDGGEVEEYRSTCPF |
| 39 | MAANPYERGPNPTDALLEARSGPFSVSEERASRLGADGFGGGTIYYPRENSDNTYGAVAIS |
| | PGYTGTQASVAWLGERIASHGFVVITIDTNTTLDQPDSRARQLNAALDYMINDSAVRSRID |
| | SSRLAVMGHSMGGGGTLRLASQRPDLKAAIPLTPWHLNKNWSSVRVPTLIIGADLDTIAPV |
| | LTHAKPFYNSLPTSISKAYLELDGATHFAPNIPNKIIGKYSVAWLKRFVDNDTRYTQFLCPGP |
| | RDGGEVEEYRSTCPF |
| 40 | MAANPYERGPNPTDALLEARSGPFSVSEENVSRLSASGFGGGTIYYPRENSSNTYGAVAIS |
| | PGYTGTQASIAWLGERIASHGFVVITIDTNTTLDQPDSRAEQLNAALDYMINRSTVRSRIDS |
| | SRLAVMGHSMGGGGTLRLASQRPDLKAAIPLTPWHLNKNWSSVTVPTLIIGADLDTIAPVA |
| | THAKPFYNSLPSSISKAYLELDGATHFAPNIPNKIIGKYSVAWLKRFVDNDTRYTQFLCPGP |
| | RDGGEVEEYRSTCPF |
| 41 | MAANPYERGPNPTDALLEARSGPFSVSEENVSRLSASGFGGGTIYYPRENSSNTYGAVAIS |
| | PGYTGTQASIAWLGERIASHGFVVITIDTNTTLDQPDSRARQLNAALNYMINRSTVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELDGATHFAPNIPNKIIGKYSVAWLKRFVDNDTRYTQFLCPGP |
| | RDGGEVEEYRSTCPF |
| 42 | MAANPYERGPDPTESSLEASSGPFSVSETSVSRLSASGFGGGTIYYPTTTSEGTYGAVAISP |
| | GYTGTQSSIAWLGPRLASHGFVVITIDTNTTFDQPDSRARQLMAALNYLVNRSSVRSRIDA |
| | SRLAVMGHSMGGGGTLRAAEQRPSLKAAIPLTPWHTDKNWSSVRVPTLIIGAENDTIAPVS |
| | THAKPFYNSLPSSISKAYLELNGASHFAPNTSNTTIGKYSISWLKRFVDNDTRYTQFLCPAP |
| | RDGSAIEEYRDTCPY |
| 43 | MAANPYERGPNPTDALLEARSGPFSVSEENVSRLSASGFGGGTIYYPRENSDNTYGAVAIS |
| | PGYTGTQASIAWLGERIASHGFVVITIDTNTTLDQPDSRARQLNAALNYMINRSTVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELDGATHFAPNIPNKIIGKYSVAWLKRFVDNDTRYTQFLCPGP |
| | RDGGEVEEYRSTCPF |
| 44 | MAENPYERGPAPTESSIEASSGPFSVSTTSVSSLSVSGFGGGTIYYPTSTSEGTFGAVAISP |
| | GYTATQSSIAWLGRRLASHGFVVITIDTNSTYDQPDSRGDQLLAALDYLTQRSSVRSRIDP |
| | SRLAVMGHSMGGGGTLEAAKDRPSLKAAIPLTPWHTDKNWSSVRVPTLIIGAENDTIAPVS |
| | SHAIPFYNSLPSSTEKAYLELNGASHFAPNSSNTTIGKYSVSWLKRFVDNDTRYSQFLCPAP |
| | SPDSAISEYRDTCPH |
| 45 | MADNPYERGPDPTESSIEASRGPFAVSETSVSRLSVSGFGGGTIYYPTTTSEGTFGAVAISP |
| | GYTGTQSSIAWLGPRLASQGFVVITIDTNTTYDQPDSRARQLLAALDYLTQRSSVRSRIDA |
| | SRLAVMGHSMGGGGTLRAAEDRPSLKAAIPLTPWHTDKNWSSVRVPTLIIGAENDTIAPVS |
| | SHAEPFYNSLPSSLPKAYLELNGASHFAPNTSNTTIAKYSISWLKRFVDNDTRYTQFLCPAP |
| | RPGSAIEEYRDTCPY |
| 46 | MADNPYERGPAPTESSIEASRGPFAVSQTSVSSLSVSGFGGGTIYYPTSTSEGTFGAVAISP |
| | GYTATQSSIAWLGPRLASQGFVVFTIDTNTRYDQPDSRGRQLLAALDYLTQRSSVRSRIDP |
| | SRLGVMGHSMGGGGTLEAAKDRPSLQAAIPLTPWNLDKNWSSVRVPTLIIGAENDTIAPV |
| | ASHSEPFYNSLPSSLDKAYLELNGASHFAPNSSNTTIAKYSISWLKRFIDNDTRYEQFLCPA |
| | PSRDSTISEYRDTCPH |
| 47 | MQANPYQRGPDPTSSSLEASSGPFSVSTTSVSRLSVSGFGGGTIYYPTNTSSGTYGAVAIS |
| | PGYTATQSSIAWLGPRLASHGFVVITIDTNSTFDQPDSRATQLMAALNYVVNSSPVRSRVD |
| | SNRLAVMGHSMGGGGTLRAAEDNPSLKAAIPLTPWHTNKNFSSVRVPTLIIGAENDTIAPV |
| | SSHAKPFYNSLPSSTSKAYLELNGASHFAPNSSNTTIGKYSVSWMKRFVDNDTRYSQFLCP |
| | APHDDSAISEYRSTCPY |
| 48 | MAENPYERGPDPTESSIEASRGPFAVSQTSVSSLSVSGFGGGTIYYPTSTSEGTFGAVAISP |
| | GYTATQSSIAWLGPRLASQGFVVFTIDTNTRYDQPASRGRQLLAALDYLTQRSSVRSRIDP |
| | SRLAVAGHSMGGGGTLEAAEDRPSLQAAIPLAPWNLDKNWSSVRVPTLIIGGESDTVAPV |
| | SSHSEPFYNSLPSSPDKAYLELNNASHFFPNTSNTTMAKYMISWLKRFVDNDTRYEQFLCP |
| | APSRDSTISEYRDTCPH |
| 49 | MAENPYERGPDPTEASIEASRGPFAISQVSVPSGSGSGFGGGTIYYPTDTSQGTFGAVAISP |
| | GFTATEASIAWLGPRLASQGFVVITIDTNSRYDQPDARADQLLAALDYLTQKSSVRSRIDP |
| | NRLAVMGHSMGGGGTLEAAENRPSLKAAIPLAPWNTNKNWSSVRVPTMIIGAQNDTIAPV |
| | GSHAEPFYNSLPASPEKAYLELKGADHFAPTSPNTTIAKYSISWLKRFVDDDTRYDQFLCPA |
| | PSSDSAISEYRDTCPH |
| 50 | MAENPYERGPAPTESSIEASRGPFAVSQTSVSSLSVSGFGGGTIYYPTSTSEGTFGAVAISP |
| | GYTATQSSIAWLGPRLASQGFVVITIDTNSRYDQPASRGDQLLAALDYLTQRSSVRSRIDP |
| | SRLAVMGHSMGGGGTLEAAKDRPSLKAAIPLTPWNTDKNWSSVRVPTLIIGAENDTIAPVS |
| | SHAEPFYNSLPSSPEKAYLELNGASHFAPNSSNTTIAKYSISWLKRFVDNDTRYEQFLCPAP |
| | SPDSTISEYRDTCPH |
| 51 | MAENPYERGPDPTESSIEATRGPFAVSQTSVSSLSVSGFGGGTIYYPTSTSEGTFGAVAISP |
| | GYTATQSSIAWLGPRLASQGFVVFTIDTNTRYDQPASRGDQLLAALDYLTQRSSVRSRIDA |
| | SRLAVMGHSMGGGGTLEAAKDRPSLQAAIPLTPWNLDKNWSEVRVPTLIIGAENDTVAPV |
| | SSHSEPFYNSLPSSPDKAYLELNGASHFAPNSSNTTIAKYSISWLKRFVDDDTRYEQFLCPA |
| | PSPDSAISEYRDTCPH |
| 52 | MQSNPYQRGPDPTRASLEASDGPFSVSTTSVSSLSVSGFGGGTIYYPTSTSSGTFGAVAIS |
| | PGYTATQSSIAWLGRRLASHGFVVITIDTNSRFDQPASRASQLLAALNYLTNSSSVRSRVD |
| | ASRLAVAGHSMGGGGTLEAAEDNPSLKAAVPLTPWNTDKNWSEVRVPTLIIGAENDTVAP |
| | VSSHAIPFYNSLPSSTEKAYVELNGASHFAPNSSNTTISKYSISWMKRFVDNDTRYSQFLC |
| | GAPNQDSAISDYRTNCPH |
| 53 | MQANPYQRGPDPTSASLEASSGPFSVSTSSVSSLSASGFGGGTIYYPTTTSSGTYGAVAIS |
| | PGYTATQSSIAWLGRRLASHGFVVITIDTNSTFDQPDSRATQLMAALNYVVNSSTVRSRVD |
| | ASRLAVMGHSMGGGGTLIAAEDNPSLKAAIPLTPWHTSKNFSSVRVPTLIIGAENDTIAPVS |
| | SHAKPFYNSLPSSTSKAYVELNGASHFAPNSSNTPIGKYSISWMKRFVDNDTRYSPFLCGA |
| | PHQGAAISEYRDNCPY |
| 54 | MADNPYERGPDPTESSIEASRGPFAVSQTSVSSLSVSGFGGGTIYYPTDTSEGTFGAVAISP |
| | GYTASQSSIAWLGPRLASQGFVVITIDTNSRYDQPDSRGRQLLAALDYLTQRSSVRSRIDP |
| | NRLAVMGHSMGGGGTLEAAEDRPSLKAAIPLTPWHTDKNWSEVRVPTMIIGAENDTIAPV |
| | SSHAEPFYNSLPSSPEKAYLELNGASHFAPNTSNTTIAKYSISWLKRFVDDDTRYDQFLCPA |
| | PSPDSAISEYRDTCPH |
| 55 | MAANPYERGPNPTDALLEARSGPFSVSEENVSRLSASGFGGGTIYYPRENSSNTYGAVAIS |
| | PGYTGTEASIAWLGERIASHGFVVITIDTITTLDQPDSRAEQLNAALNHMINRSTVRSRIDS |
| | SRLAVMGHSMGGGGTLRLASQRPDLKAAIPLTPWHLNKNWSSVTVPTLIIGADLDTIAPVA |
| | THAKPFYNSLPSSISKAYLELDGATHVAPNIPNKIIGKYSVAWLKRFVDNDTRYTQFLCPGP |
| | RDGGEVEEYRSTCPF |
| 56 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELDGATHVAPNIPNKIIGKYSVAWLKRFVDNDTRYTQFLCPGP |
| | RDGGEVEEYRSTCPF |
| 57 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALNYMINRSTVRSRIDS |
| | SRLAVMGHSMGGGGTLRLASQRPDLKAAIPLTPWHLNKNWSSVTVPTLIIGADLDTIAPVA |
| | THAKPFYNSLPSSISKAYLELDGATHVAPNIPNKIIGKYSVAWLKRFVDNDTRYTQFLCPGP |
| | RDGGEVEEYRSTCPF |
| 58 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALNYMINRSTVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELDGATHVAPNIPNKIIGKYSVAWLKRFVDNDTRYTQFLCPGP |
| | RDGGEVEEYRSTCPF |
| 59 | MAANPYERGPNPTDALLEARSGPFSVSEENVSRLSASGFGGGTIYYPRENSSNTYGAVAIS |
| | PGYTGTEASIAWLGERIASHGFVVITIDTITTLDQPDSRAEQLNAALNHMINRSTVRSRIDS |
| | SRLAVMGHSMGGGGTLRLASQRPDLKAAIPLTPWHLNKNWSSVTVPTLIIGADLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 60 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 61 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALNYMINRSTVRSRIDS |
| | SRLAVMGHSMGGGGTLRLASQRPDLKAAIPLTPWHLNKNWSSVTVPTLIIGADLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 62 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALNYMINRSTVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 63 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALNYMINRSTVRSRIDS |
| | SRLAVMGHSMGGGGTLRLASQRPDLKAAIPLTPWHLNKNWSSVTVPTLIIGACLDTIAPVA |
| | THAKPFYNSLPSSISKAYLELDGATHFAPNIPNKIIGKYSVAWLKRFVDNDTRYTQFLCPGP |
| | RDGGEVCEYRSTCPF |
| 64 | MAANPYERGPNPTDALLEARSGPFSVSEENVSRLSASGFGGGTIYYPRENSSNTYGAVAIS |
| | PGYTGTEASIAWLGERIASHGFVVITIDTITTLDQPDSRAEQLNAALNHMINRSTVRSRIDS |
| | SRLAVMGHSMGGGGTLRLASQRPDLKAAIPLTPWHLNKNWSSVTVPTLIIGACLDTIAPVA |
| | THAKPFYNSLPSSISKAYLELCGATHFAPNIPNKIIGKYSVAWLKRFVDNDTRYTQFLCPGPR |
| | DGGEVEEYRSTCPF |
| 65 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGACLDTIAPVA |
| | THAKPFYNSLPSSISKAYLELCGATHFAPNIPNKIIGKYSVAWLKRFVDNDTRYTQFLCPGPR |
| | DGGEVEEYRSTCPF |
| 66 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALNYMINRSTVRSRIDS |
| | SRLAVMGHSMGGGGTLRLASQRPDLKAAIPLTPWHLNKNWSSVTVPTLIIGACLDTIAPVA |
| | THAKPFYNSLPSSISKAYLELCGATHFAPNIPNKIIGKYSVAWLKRFVDNDTRYTQFLCPGPR |
| | DGGEVEEYRSTCPF |
| 67 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALNYMINRSTVRSRIDS |
| | SRLAVMGHSMGGGGTLRLASQRPDLKAAIPLTPWHLNKNWSSVRVPTLIIGACLDTIAPVA |
| | THAKPFYNSLPSSISKAYLELCGATHFAPNIPNKIIGKYSVAWLKRFVDNDTRYTQFLCPGPR |
| | DGGEVEEYRSTCPF |
| 68 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGARLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 69 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALNYMINRSTVRSRIDS |
| | SRLAVMGHSMGGGGTLRLASQRPDLKAAIPLTPWHLNKNWSSVTVPTLIIGARLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 70 | MAANPYERGPNPTDALLEARSGPFSVSEENVSRLSASGFGGGTIYYPRENSENTYGAVAISP |
| | GYTGTQASIAWLGERIASHGFVVITIDTNTTLDQPDSRARQLNAALNYMINRSTVRSRIDS |
| | SRLAVMGHSMGGGGTLRLASQRPDLKAAIPLTPWHLNKNWSSVRVPTLIIGARLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 71 | AANPYERGPNPTDALLEARSGPFSVSEERASRLSADGFGGGTIYYPRENSENTYGAVAISPG |
| | YTGTQASMAWLGERIASHGFVVITIDTNTTLDQPDSRARQLNAALDHMINDSAVRSRIDS |
| | SRLAVMGHSMGGGGTLRLASQRPDLKAAIPLTPWHLNKNWSNVRVPTLIIGADLDTIAPVL |
| | THAKPFYNSLPTSISKAYLELCGATHFAPNIPNKIIGKYSVAWLKRFVDNDTRYTQFLCPGPR |
| | DGGEVCEYRSTGPF |
| 72 | AANPYERGPNPTDALLEARSGPFSVSEERASRLSADGFGGGTIYYPRENSENTYGAVAISPG |
| | YTGTQASMAWLGERIASHGFVVITIDTNTTLDQPDSRARQLNAALNHMINDSAVRSRIDSS |
| | RLAVMGHSMGGGGTLRLASQRPDLKAAIPLTPWHLNKNWSSVRVPTLIIGADLDTIAPVLT |
| | HAKPFYNSLPTSISKAYLELCGATHFAPNIPNKIIGKYSVAWLKRFVDNDTRYTQFLCPGPR |
| | DGGEVCEYRSTGPF |
| 73 | AANPYERGPNPTNALLEARSGPFSVSEENVSRLSADGFGGGTIYYPRENSENTYGAVAISPG |
| | YTGTQASIAWLGERIASHGFVVITIDTNTTLDQPDSRARQLNAALDYMINDSAVRSRIDSS |
| | RLAVMGHSMGGGGTLRLASQRPDLKAAIPLTPWHLNKNWSSVRVPTLIIGADLDTIAPVAT |
| | HAKPFYNSLPSSISKAYLELCGATHFAPNIPNKIIGKYSVAWLKRFVDNDTRYTQFLCPGPR |
| | DGGEVCEYRSTCPF |
| 74 | AANPYERGPNPTDALLEARSGPFSVSEENVSRLSADGFGGGTIYYPRENSENTYGAVVISP |
| | GYTGTQASIAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 75 | AANPYERGPNPTDALLEARSGPFSVSEERVSRLSADGFGGGTIYYPRENSENTYGAVAISPG |
| | YTGTQASMAWLGERIASHGFVVITIDTNTTLDQPDSRARQLNAALDHMINDSAVRSRIDS |
| | SRLAVMGHSMGGGGTLRLASQRPDLKAAIPLTPWHLNKNWSSVRVPTLIIGADLDTIAPVL |
| | THAKPFYNSLPTSISKAYLELCGATHFAPNIPNKIIGKYSVAWLKRFVDNDTRYTQFLPPGPR |
| | DGGEVCEYRSTGPF |
| 76 | MANPYERGPNPTEALLEARSGPFSVSEERASRLGADGFGGGTIYYPRENSENTYGAVAISP |
| | GYTGTQASVAWLGERIASHGFVVITIDTNTTLDQPDSRARQLNAALDYMLNDSAVRSRIDS |
| | SRLAVMGHSMGGGGTLRLASQRPDLKAAIPLTPWHLNKSWSNVQVPTLIIGADLDTIAPVL |
| | THAEPFYNSIPTSTSKAYLELDGATHFAPNITNKTIGMYSVAWLKRFVDEDTRYTQFLCPGP |
| | RTGSDVEEYRSTCPF |
| 77 | AANPYERGPNPTDALLEARSGPFSVSEETVSRLSASGFGGGTIYYPRENSENTYGAVAISPG |
| | YTGTQASIAWLGERIASHGFVVITIDTNTTLDQPDSRARQLNAALDYMINDSAVRSRIDSS |
| | RLAVMGHSMGGGGTLRLASQRPDLKAAIPLTPWHLNKNWSSVRVPTLIIGADLDTIAPVAT |
| | HAKPFYNSLPSSISKAYLELCGATHFAPNIPNKIIGKYSVAWLKRFVDNDTRYTQFLCPGPR |
| | DGGDVCEYRSTCPF |
| 78 | AANPYERGPNPTDALLEARSGPFSVSEENVSRLSADGFGGGTIYYPRENSENTYGAVAISP |
| | GYTGTQASIAWLGERIASHGFVVITIDTNTTLDQPDSRARQLNAALDYMINDSAVRSRIDS |
| | SRLAVMGHSMGGGGTLRLASQRPDLKAAIPLTPWHLNKTWSSVRVPTLIIGADLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 79 | AANPYERGPNPTDALLEARRGPFSVSEENVSRLSADGFGGGTIYYPRENSENTYGAVAISP |
| | GYTGTQASIAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 80 | AANPYERGPNPTDALLEARSGPFSVSEERASRLGADGFGGGTIYYPRENSENTYGAVAISP |
| | GYTGTQASMAWLGERIASHGFVVITIDTNTTLDQPDSRARQLNAALDHMINDSAVRSRID |
| | SSRLAVMGHSMGGGGTLRLASQRPDLKAAIPLTPWHLNKNWSSVRVPTLIIGADLDTIAPV |
| | LTHAKPFYNSLPTSISKAYLELCGATHFAPNIPNKIIGKYSVAWLKRFVDNDTRYTQFLPPGP |
| | RTGGEVCEYRSTGPF |
| 81 | MANPYERGPNPTDALLEARSGPFSVSEERASRLGADGFGGGTIYYPRENSENTYGAVAISP |
| | GYTGTQASMAWLGERIASHGFVVITIDTNTTLDQPDSRARQLNAALDHMINDSAVRSRID |
| | SSRLAVMGHSMGGGGTLRLASQRPDLKAAIPLTPWHLNKNWSNVRVPTLIIGADLDTIAPV |
| | LTHAKPFYNSLPTSISKAYLELDGATHFAPNIPNKIIGKYSVAWLKRFVDNDTRYTQFLPPGP |
| | RTGGEVSEYRSTGPF |
| 82 | MANPYERGPNPTDALLEARSGPFSVSEERASRLGADGFGGGTIYYPRENSENTYGAVAISP |
| | GYTGTQASVAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVTVPTLIIGADLDTIAPVL |
| | THARPFYNSLPTSISKAYLELDGATHFAPNIPNKIIGKYSVAWLKRFVDNDTRYTQFLCPGPR |
| | DGGEVEEYRSTCPF |
| 83 | MANPYERGPNPTEALLEARSGPFSVSEERASRLGADGFGGGTIYYPRENSDNTYGAVAISP |
| | GYTGTQASVAWLGERIASHGFVVITIDTNTTLDQPDSRARQLNAALDYMLNDSAVRSRIDS |
| | SRLAVMGHSMGGGGTLRLASQRPDLKAAIPLTPWHLNKSWSNVQVPTLIIGADLDTIAPVL |
| | THAEPFYNSIPTSTSKAYLELDGATHFAPNITNKTIGMYSVAWLKRFVDEDTRYTQFLCPGP |
| | RTGSDVEEYRSTCPF |
| 84 | AANPYERGPNPTDALLEARSGPFSVSEENVSRLSADGFGGGTIYYPRENSENTYGAVAISP |
| | GYTGTQASMAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 85 | AANPYERGPNPTDALLEARSGPFSVSEENVSRLSADGFGGGTIYYPRENSENTYGAVAISP |
| | GYTGTQASIAWLGERIASHGFVVITIDTNTTLDQPDSRAKQLNAALDYMINDSAVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 86 | AANPYERGPNPTDALLEARSGPFSVSEENVSRLSADGFGGGTIYYPRENSENTYGAVAISP |
| | GYTGTQASIAWLGERIASHGFVVITIDTNTTLDQPDSRARQLNAALDYMINDSAVRSRIDS |
| | SRLGVMGHSMGGGGTLRLASQRPDLKAAIPLTPWHLNKNWSSVRVPTLIIGADLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 87 | MANPYERGPNPTDALLEARSGPFSVSEERASRLGADGFGGGTIYYPRENSDNTYGAVAISP |
| | GYTGTQASMAWLGERIASHGFVVITIDTNTTLDQPDSRARQLNAALDHMINDSTVRSRID |
| | SSRLAVMGHSMGGGGTLRLASQRPDLKAAIPLTPWHLNKNWSNVRVPTLIIGADLDTIAPV |
| | LTHAKPFYNSLPTSISKAYLELCGATHFAPNIPNKTIGKYSVAWLKRFVDNDTRYTQFLPPGP |
| | RTGGEVCEYRSTGPF |
| 88 | AANPYERGPNPTDALLEARSGPFSVSEERASRLSADGFGGGTIYYPRENSENTYGAVAISPG |
| | YTGTQASMAWLGERIASHGFVVITIDTNTTLDQPDSRARQLNAALDHMINDSAVRSRIDS |
| | SRLAVMGHSMGGGGTLRLASQRPDLKAAIPLTPWHLNKNWSSVRVPTLIIGADLDTIAPVL |
| | THAKPFYNSLPTSISKAYLELCGATHFAPNIPNKIIGKYSVAWLKRFVDNDTRYTQFLPPGPR |
| | DGGEVCEYRSTGPF |
| 89 | MANPYERGPNPTEALLEARSGPFSVSEERASRLGADGFGGGTIYYPRENSENTYGAVAISP |
| | GYTGTQASVAWLGERIASHGFVVITIDTNTTLDQPDSRARQLNAALDYMLNDSAVRSRIDS |
| | SRLAVMGHSMGGGGTLRLASQRPDLKAAIPLTPWHLNKSWSNVTVPTLIIGADLDTIAPVL |
| | THAEPFYNSIPTSTSKAYLELDGATHFAPNITNKTIGMYSVAWLKRFVDEDTRYTQFLCPGP |
| | RTGSDVEEYRSTCPF |
| 90 | MANPYERGPNPTEALLEARSGPFSVSEERASRLGADGFGGGTIYYPRENSSNTYGAVAISP |
| | GYTGTQASVAWLGERIASHGFVVITIDTNTTLDQPDSRARQLNAALDYMLNDSAVRSRIDS |
| | SRLAVMGHSMGGGGTLRLASQRPDLKAAIPLTPWHLNKSWSNVQVPTLIIGADLDTIAPVL |
| | THAEPFYNSIPTSTSKAYLELDGATHFAPNITNKTIGMYSVAWLKRFVDEDTRYTQFLCPGP |
| | RTGSDVEEYRSTCPF |
| 91 | AENPYERGPDPTESSIEASRGPFAVSETSVSRLSVSGFGGGTIYYPTSTSEGTFGAVAISPG |
| | YTATQSSIAWLGPRLASQGFVVITIDTNSRYDQPDSRARQLLAALDYLTNSSSVRSRIDPSR |
| | LAVMGHSMGGGGTLQAAEDRPSLKAAIPLTPWHTDKNWSSVRVPTLIIGAENDTVAPVSS |
| | HAKPFYNSLPSSPEKAYLELNGASHFAPNSSNTTIAKYSIAWLKRFVDDDTRYEQFLCPAPS |
| | TDSAISEYRSTCPY |
| 92 | AANPYERGPNPTDALLEARSGPFSVSEENVSRLSASGFGGGTIYYPRENSENTYGAVAISPG |
| | YTGTQASMAWLGERIASHGFVVITIDTNTTLDQPDSRARQLNAALDYMINDSAVRSRIDSS |
| | RLAVMGHSMGGGGTLRLASQRPDLKAAIPLTPWHLNKNWSSVRVPTLIIGADLDTIAPVAT |
| | HAKPFYNSLPSSISKAYLELCGATHFAPNIPNKIIGKYSVAWLKRFVDNDTRYTQFLCPGPR |
| | DGGDVCEYRSTCPF |
| 93 | AANPYERGPNPTDALLEARSGPFSVSEESVSRLSASGFGGGTIYYPRENSENTYGAVAISPG |
| | YTGTQASIAWLGERIASHGFVVITIDTNTTLDQPDSRARQLNAALDYMINDSAVRSRIDSS |
| | RLAVMGHSMGGGGTLRLASQRPDLKAAIPLTPWHLNKNWSSVRVPTLIIGADLDTIAPVAT |
| | HAKPFYNSLPSSISKAYLELCGATHFAPNIPNKIIGKYSVAWLKRFVDNDTRYTQFLCPGPR |
| | DGGDVCEYRSTCPF |
| 94 | AANPYERGPNPTDALLEARSGPFSVSEENVSRLSADGFGGGTIYYPRENSENTYGAVAISP |
| | GYTGTQASIAWLGERIASHGFVVITIDTNTTLDQPDSRARQLNAALDYMINDSAVRSMIDS |
| | SRLAVMGHSMGGGGTLRLASQRPDLKAAIPLTPWHLNKNWSSVRVPTLIIGADLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 95 | AANPYERGPNPTDALLEARSGPFSVSEENVSRLSASGFGGGTIYYPRENSENTYGAVAISPG |
| | YTGTQASIAWLGERIASHGFVVITIDTNTTLDQPDSRARQLNAALDYMINDSAVRSRIDSS |
| | RLAVMGHSMGGGGTLRLASQRPDLKAAIPLTPWHLNKNWNSVRVPTLIIGADLDTIAPVAT |
| | HAKPFYNSLPSSISKAYLELCGATHFAPNIPNKIIGKYSVAWLKRFVDNDTRYTQFLCPGPR |
| | DGGDVCEYRSTCPF |
| 96 | AANPYERGPNPTDALLEARSGPFSVSEENVSRLSADGFGGGTIYYPRENSENTYGAVAISP |
| | GYTGTQASIAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELCGATHFAPNIPNKIIGKYSVAWLKRFVDNDTRYTQFLCPGPR |
| | DGGDVCEYRSTCPF |
| 97 | AANPYERGPNPTNALLEARSGPFSVSEENVSRLSASGFGGGTIYYPRENSENTYGAVAISPG |
| | YTGTQASIAWLGERIASHGFVVITIDTNTTLDQPDSRARQLNAALDYMINDSAVRSRIDSS |
| | RLAVMGHSMGGGGTLRLASQRPDLKAAIPLTPWHLNKNWSSVRVPTLIIGADLDTIAPVAT |
| | HAKPFYNSLPSSISKAYLELCGATHFAPNIPNKIIGKYSVAWLKRFVDNDTRYTQFLCPGPR |
| | DGGDVCEYRSTCPF |
| 98 | AANPYERGPNPTDALLEARSGPFSVSEENVSRLSASGFGGGTIYYPRENSENTYGAVAISPG |
| | YTGTQASIAWLGERIASHGFVVITIDTNTTLDQPDSRARQLNAALDYMINDSAVRSRIDSS |
| | RLAVMGHSMGGGGTLRLASQRPDLKAAIPLTPWHLNKNWSSVRVPTLIIGADLDTIAPVAT |
| | HAKPFYNSLPSSISKAYLELCGGTHFAPNIPNKIIGKYSVAWLKRFVDNDTRYTQFLCPGPR |
| | DGGDVCEYRSTCPF |
| 99 | QANPYERGPNPTDALLEARSGPFSVSEENVSRLSADGFGGGTIYYPRENSENTYGAVAISP |
| | GYTGTQASIAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 100 | AANPYERGPDPTESSLEASSGPFAVSETSVSRLSVSGFGGGTIYYPTSTSSGTYGAVAISPG |
| | YTATQSSIAWLGPRLASHGFVVITIDTNSRFDQPDSRARQLLAALNYLVNSSSVRSRIDSS |
| | RLAVMGHSMGGGGTLRAAEDRPSLKAAIPLTPWHTDKNWSSVTVPTLIIGAENDTVAPVS |
| | SHAKPFYNSLPSSTPKAYLELNGASHFAPNSSNTTIGKYSIAWLKRFVDNDTRYSQFLCPAP |
| | SDDSAISEYRSTCPY |
| 101 | MANPYERGPNPTDALLEARSGPFSVSEERASRLGADGFGGGTIYYPRENSENTYGAVAISP |
| | GYTGTQASVAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGADLDTIAPVL |
| | THARPFYNSLPTSISKAYLELDGATHFAPNIPNKIIGKYSVAWLKRFVDNDTRYTQFLCPGPR |
| | DGGEVEEYRSTCPF |
| 102 | AANPYERGPNPTDALLEARSGPFSVSEENVSRLSADGFGGGTIYYPRENSENTYGAVAISP |
| | GYTGTQASIAWLGERIASHGFVVITIDTNTTLDQPDSRARQLNAALDYMINDSAVRSRIDS |
| | SRLAVMGHSMGGGGTLRLASQRPDLKAAIPLTPWHLNKNWNSVRVPTLIIGADLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 103 | AANPYERGPNPTDALLEARSGPFSVSEENVSRLSADGFGGGTIYYPRENSTNTYGAVAISPG |
| | YTGTQASIAWLGERIASHGFVVITIDTNTTLDQPDSRARQLNAALDYMINDSAVRSRIDSS |
| | RLAVMGHSMGGGGTLRLASQRPDLKAAIPLTPWHLNKNWSSVRVPTLIIGADLDTIAPVAT |
| | HAKPFYNSLPSSISKAYLELCGATHFAPNIPNKIIGKYSVAWLKRFVDNDTRYTQFLCPGPR |
| | DGGEVCEYRSTCPF |
| 104 | AANPYERGPNPTDALLEARSGPFSVSEERVSRLSADGFGGGTIYYPRENSENTYGAVAISPG |
| | YTGTQASMAWLGERIASHGFVVITIDTNTTLDQPDSRARQLNAALNHMINDSAVRSRIDSS |
| | RLAVMGHSMGGGGTLRLASQRPDLKAAIPLTPWHLNKNWSSVRVPTLIIGADLDTIAPVLT |
| | HAKPFYNSLPTSISKAYLELCGATHFAPNIPNKIIGKYSVAWLKRFVDNDTRYTQFLPPGPRD |
| | GGEVCEYRSTGPF |
| 105 | AENPYERGPDPTESSIEALRGPFAVSEESVSRLSVSGFGGGTIYYPTDTSEGTFGAVAISPG |
| | YTGTQSSMAWLGPRIASQGFVVFTIDTNTTYDQPDSRARQLQAALDYLVEDSSVRDRIDP |
| | NRLGVMGHSMGGGGTLRAAEDRPDLKAAIPLTPWHLNKNWSSVRVPTLIIGAENDTIAPV |
| | RTHAEPFYESLPSSLDKAYLELDGASHFAPNISNTTIAKYSISWLKRFVDDDTRYEQFLCPPP |
| | RTGSEISEYRSTCPF |
| 106 | AANPYERGPNPTEALLEARSGPFSVSEERASRLGADGFGGGTIYYPRENSDNTYGAVAISP |
| | GYTGTQASVAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGADLDTIAPVL |
| | THAEPFYNSLPTSISKAYLELDGATHFAPNITNKTIGKYSVAWLKRFVDEDTRYTQFLCPGP |
| | RTGSDVEEYRSTCPF |
| 107 | AANPYERGPNPTDALLEARSGPFSVSEERASRLGADGFGGGTIYYPRENSDNTYGAVAISP |
| | GYTGTQASVAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGADLDTIAPVL |
| | THAKPFYNSLPTSISKAYLELDGATHFAPNIPNKIIGKYSVAWLKRFVDNDTRYTQFLCPGPR |
| | DGGEVEEYRSTCPF |
| 108 | AANPYERGPNPTDALLEARSGPFSVSEERVSRLSADGFGGGTIYYPRENSENTYGAVAISPG |
| | YTGTQASIAWLGERIASHGFVVITIDTNTTLDQPDSRARQLNAALDYMINDSAVRSRIDSS |
| | RLAVMGHSMGGGGTLRLASQRPDLKAAIPLTPWHLNKNWSSVRVPTLIIGADLDTIAPVAT |
| | HAKPFYNSLPSSISKAYLELCGATHFAPNIPNKIIGKYSVAWLKRFVDNDTRYTQFLCPGPR |
| | DGGEVCEYRSTCPF |
| 109 | AANPYERGPNPTDALLEARSGPFSVSEENVSRLSADGFGGGTIYYPRENSENTYGAVAISP |
| | GYTGTQASIAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELCGGTHFAPNIPNKIIGKYSVAWLKRFVDNDTRYTQFLCPGP |
| | RDGGEVCEYRSTCPF |
| 110 | AANPYERGPNPTDALLEARSGPFSVSEENVSRLSADGFGGGTIYYPRENSENTYGAVAISP |
| | GYTGTQASIAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELCGATHFAPNIPNKIIGKYSVAWLKRFVDNDTRYTQFLCPGPR |
| | DGREVCEYRSTCPF |
| 111 | AANPYERGPNPTDALLEARSGPFSVSEENVSRLSADGFGGGTIYYPRENSENTYGAVAISP |
| | GYTGTQASIAWLGERIASHGFVVITIDTNTTLDQPDSRARQLNAALDYMINDSAVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELCGATHFTPNIPNKIIGKYSVAWLKRFVDNDTRYTQFLCPGPR |
| | DGGEVCEYRSTCPF |
| 112 | AANPYERGPNPTDALLEARSGPFSVSEERVSRLGADGFGGGTIYYPRENSENTYGAVAISP |
| | GYTGTQASMAWLGERIASHGFVVITIDTNTTLDQPDSRARQLNAALDHMINDSAVRSRID |
| | SSRLAVMGHSMGGGGTLRLASQRPDLKAAIPLTPWHLNKNWSSVRVPTLIIGADLDTIAPV |
| | LTHAKPFYNSLPTSISKAYLELCGATHFAPNIPNKIIGKYSVAWLKRFVDNDTRYTQFLPPGP |
| | RDGGEVCEYRSTGPF |
| 113 | AANPYERGPDPTESSLEASSGPFSVSETSVSRLSVSGFGGGTIYYPTDTSSGTYGAVAISPG |
| | YTATQSSIAWLGPRLASHGFVVITIDTNSTFDQPDSRARQLMAALDYLVNSSSVRSRIDSS |
| | RLAVMGHSMGGGGTLRAAEDNPSLKAAIPLTPWHTDKNWSSVRVPTLIIGAENDTVAPVS |
| | SHAKPFYNSLPSSTPKAYLELNGASHFAPNSSNTTIGKYSVSWLKRFVDNDTRYSQFLCPA |
| | PHDDSAISEYRSTCPY |
| 114 | AANPYERGPNPTEALLEARSGPFSVSEERASRLGADGFGGGTIYYPRENSDNTYGAVAISP |
| | GYTGTQASVAWLGERIASHGFVVITIDTNTTLDQPDSRARQLNAALDYMLNDSAVRSRIDS |
| | SRLAVMGHSMGGGGTLRLASQRPDLKAAIPLTPWHLNKSWSNVQVPTLIIGADLDTIAPVL |
| | THAEPFYNSIPTSTSKAYLELDGATHFAPNITNKTIGMYSVAWLKRFVDEDTRYTQFLCPGP |
| | RTGSDVEEYRSTCPF |
| 115 | AANPYERGPNPTDALLEARSGPFSVSEESVSRLSADGFGGGTIYYPRENSENTYGAVAISPG |
| | YTGTQASIAWLGERIASHGFVVITIDTNTTLDQPDSRARQLNAALDYMINDSAVRSRIDSS |
| | RLAVMGHSMGGGGTLRLASQRPDLKAAIPLTPWHLNKNWSSVRVPTLIIGADLDTIAPVAT |
| | HAKPFYNSLPSSISKAYLELCGATHFAPNIPNKIIGKYSVAWLKRFVDNDTRYTQFLCPGPR |
| | DGGEVCEYRSTCPF |
| 116 | AANPYERGPNPTDALLEARSGPFSVSEENVSRLSASGFGGGTIYYPRENSENTYGAVAISPG |
| | YTGTQASIAWLGERIASHGFVVITIDTNTTLDQPDSRARQLNAALDYMINDSAVRSRIDSS |
| | RLAVMGHSMGGGGTLRLASQRPDLKAAIPLTPWHLNKTWSSVRVPTLIIGADLDTIAPVAT |
| | HAKPFYNSLPSSISKAYLELCGATHFAPNIPNKIIGKYSVAWLKRFVDNDTRYTQFLCPGPR |
| | DGGDVCEYRSTCPF |
| 117 | AANPYERGPNPTDALLEARSGPFTVSEENVSRLSADGFGGGTIYYPRENSENTYGAVAISPG |
| | YTGTQASIAWLGERIASHGFVVITIDTNTTLDQPDSRARQLNAALDYMINDSAVRSRIDSS |
| | RLAVMGHSMGGGGTLRLASQRPDLKAAIPLTPWHLNKNWSSVRVPTLIIGADLDTIAPVAT |
| | HAKPFYNSLPSSISKAYLELCGATHFAPNIPNKIIGKYSVAWLKRFVDNDTRYTQFLCPGPR |
| | DGGEVCEYRSTCPF |
| 118 | AANPYERGPNPTDALLEARSGPFSVSEENVSRLSADGFGGGTIYYPRENSENTYGAVAISP |
| | GYTGTQASIAWLGERIASHGFVVITIDTNTTLDQPDSRARQLNAALDHMINDSAVRSRIDS |
| | SRLAVMGHSMGGGGTLRLASQRPDLKAAIPLTPWHLNKNWSSVRVPTLIIGADLDTIAPVA |
| | THAKPFYNSLPSSISKAYLELCGATHFAPNIPNKIIGKYSVAWLKRFVDNDTRYTQFLCPGPR |
| | DGGEVCEYRSTCPF |
| 119 | GPXPTXXXXEAXXGPFXXXXXXXXXXXXXGFGGGTIYYPXXXSXXXXGAVXI |
| | SPGXTXXXXSXAWLGXRXASXGFVVXTIDTXXXXDQPXXRXXQLXAALXXXXXXSXVRXX |
| | XDXXRLXVXGHSMGGGGTLXXAXXXPXLXAAXPLXXWXXXKXXXXVXVPTXXIGXXXDTX |
| | APVXXHXXPFYXSXPXXXXKAYXELXXXXHXXPXXXNXXXXXYXXXWXKRFXDXDTRYXX |
| | FLXXXPXXXXXXXXYRXXXPX |
| | wherein X can be any amino acid. |

TABLE 2
Efficiency of three exemplary DOCTase polypeptides

| SEQ | MOCT | TPA | Accumulative |
|---|---|---|---|
| ID | Concentration | Concentration | MOCT/TPA |
| NO | (peak area) | (peak area) | (peak area) |
| 6 | 33.33000946 | 157.8963776 | 191.226387 |
| 7 | 128.8890686 | 184.8075409 | 313.6966095 |
| 8 | 71.81786346 | 180.6810913 | 252.4989548 |

TABLE 3
Efficiency of three exemplary MOCTase polypeptides

| SEQ | |
|---|---|
| ID | TPA Concentration |
| NO | (peak area) |
| 9 | 7304.587011 |
| 10 | 7310.312197 |
| 11 | 6510.802634 |

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### REFERENCES

Almagro Armenteros, Jose Juan, Konstantinos D. Tsirigos, Casper Kaae Sonderby, Thomas Nordahl Petersen, Ole Winther, Soren Brunak, Gunnar von Heijne, and Henrik Nielsen. 2019. “SignalP 5.0 Improves Signal Peptide Predictions Using Deep Neural Networks.” Nature Biotechnology 37 (4): 420-23.
Anisimova, M., and O. Gascuel. 2006. “Approximate Likelihood-Ratio Test for Branches: A Fast, Accurate, and Powerful Alternative.” Systematic Biology 55 (4). https://doi.org/10.1080/10635150600755453.
Austin, Harry P., Mark D. Allen, Bryon S. Donohoe, Nicholas A. Rorrer, Fiona L. Kearns, Rodrigo L. Silveira, Benjamin C. Pollard, et al. 2018. “Characterization and Engineering of a Plastic-Degrading Aromatic Polyesterase.” Proceedings of the National Academy of Sciences of the United States of America 115 (19): E4350-57.
Castro-Ochoa, D., C. Peña-Montes, A. González-Canto, A. Alva-Gasca, R. Esquivel-Bautista, A. Navarro-Ocaña, and A. Farrés. 2012. “ANCUT2, an Extracellular Cutinase from Aspergillus Nidulans Induced by Olive Oil.” Applied Biochemistry and Biotechnology 166 (5). https://doi.org/10.1007/s12010-011-9513-7.
“Chapter 4 Cutinases:: Properties and Industrial Applications.” 2009. In Advances in Applied Microbiology, 66:77-95. Academic Press.
Cui, Yinglu, Yanchun Chen, Xinyue Liu, Saijun Dong, Yu'e Tian, Yuxin Qiao, Ruchira Mitra, et al. 2021. “Computational Redesign of a PETase for Plastic Biodegradation under Ambient Condition by the GRAPE Strategy.” ACS Catalysis. https://doi.org/10.1021/acscatal.0c05126.
“Cutinases from Thermophilic Bacteria (actinomycetes): From Identification to Functional and Structural Characterization.” 2021. In Methods in Enzymology, 648:159-85. Academic Press.
Ettinger, William F., Sushil K. Thukral, and Pappachan E. Kolattukudy. 1987. “Structure of Cutinase Gene, cDNA, and the Derived Amino Acid Sequence from Phytopathogenic Fungi.” Biochemistry. https://doi.org/10.1021/bi00398a052.
Fu, Limin, Beifang Niu, Zhengwei Zhu, Sitao Wu, and Weizhong Li. 2012. “CD-HIT: Accelerated for Clustering the next-Generation Sequencing Data.” Bioinformatics 28 (23): 3150-52.
Furukawa, Makoto, Norifumi Kawakami, Atsushi Tomizawa, and Kenji Miyamoto. 2019. “Efficient Degradation of Poly(ethylene Terephthalate) with Thermobifida fusca Cutinase Exhibiting Improved Catalytic Activity Generated Using Mutagenesis and Additive-Based Approaches.” Scientific Reports 9 (1): 16038.
Furukawa, Ryutaro, Wakako Toma, Koji Yamazaki, and Satoshi Akanuma. 2020. “Ancestral Sequence Reconstruction Produces Thermally Stable Enzymes with Mesophilic Enzyme-like Catalytic Properties.” Scientific Reports 10 (1): 15493.
Gemeren, I. A. van, A. Beijersbergen, C. A. M. J. J. van den Hondel, and C. T. Verrips. 1998. “Expression and Secretion of Defined Cutinase Variants by Aspergillus awamori.” Applied and Environmental Microbiology 64 (8): 2794.
Gumulya, Yosephin, Jong-Min Baek, Shun-Jie Wun, Raine E. S. Thomson, Kurt L. Harris, Dominic J. B. Hunter, James B. Y. Behrendorff, et al. 2018. “Engineering Highly Functional Thermostable Proteins Using Ancestral Sequence Reconstruction.” Nature Catalysis. https://doi.org/10.1038/s41929-018-0159-5.
Hoang, Diep Thi, Olga Chernomor, Arndt von Haeseler, Bui Quang Minh, and Le Sy Vinh. 2018. “UFBoot2: Improving the Ultrafast Bootstrap Approximation.” Molecular Biology and Evolution 35 (2): 518-22.
Ion, Sabina; Voicea, Stefania; Sora, Cristina; Gheorghita, Giulia; Tudorache, Madalina; Parvulescu, Vasile I. 2021. “Sequential biocatalytic decomposition of BHET as valuable intermediator of PET recycling strategy” Catalysis Today 366:177-184.
Kalyaanamoorthy, Subha, Bui Quang Minh, Thomas K. F. Wong, Arndt von Haeseler, and Lars S. Jermiin. 2017. “ModelFinder: Fast Model Selection for Accurate Phylogenetic Estimates.” Nature Methods 14 (6): 587-89.
Katoh, Kazutaka, and Daron M. Standley. 2013. “MAFFT Multiple Sequence Alignment Software Version 7: Improvements in Performance and Usability.” Molecular Biology and Evolution 30 (4): 772-80.
Kleeberg, I., K. Welzel, J. Vandenheuvel, R-J Müller, and W-D Deckwer. 2005. “Characterization of a New Extracellular Hydrolase from Thermobifida fusca Degrading Aliphatic-Aromatic Copolyesters.” Biomacromolecules 6 (1): 262-70.
Martinez, Chrislaine, Pieter De Geus, Marc Lauwereys, Gaston Matthyssens, and Christian Cambillau. 1992. “Fusarium solani Cutinase Is a Lipolytic Enzyme with a Catalytic Serine Accessible to Solvent.” Nature 356 (6370): 615-18.
Ma, Yuan, Mingdong Yao, Bingzhi Li, Mingzhu Ding, Bo He, Si Chen, Xiao Zhou, and Yingjin Yuan. 2018. “Enhanced Poly(ethylene Terephthalate) Hydrolase Activity by Protein Engineering.” Engineering. https://doi.org/10.1016/j.eng.2018.09.007.
Minh, Bui Quang, Heiko A. Schmidt, Olga Chernomor, Dominik Schrempf, Michael D. Woodhams, Arndt von Haeseler, and Robert Lanfear. 2020. “Corrigendum to: IQ-TREE 2: New Models and Efficient Methods for Phylogenetic Inference in the Genomic Era.” Molecular Biology and Evolution 37 (8): 2461.
Nyyssölä, Antti. 2015. “Which Properties of Cutinases Are Important for Applications?” Applied Microbiology and Biotechnology 99 (12): 4931-42.
Paradis, E., J. Claude, and K. Strimmer. 2004. “APE: Analyses of Phylogenetics and Evolution in R Language.” Bioinformatics. https://doi.org/10.1093/bioinformatics/btg412.
Perez-Garcia, Pablo, Stefanie Kobus, Christoph G. W. Gertzen, Astrid Hoeppner, Nicholas Holzscheck, Christoph Heinrich Strunk, Harald Huber, et al. 2021. “A Promiscuous Ancestral Enzyme's Structure Unveils Protein Variable Regions of the Highly Diverse Metallo-β-Lactamase Family.” Communications Biology 4 (1): 132.
Ribitsch, Doris, Enrique Herrero Acero, Katrin Greimel, Inge Eiteljoerg, Eva Trotscha, Giuliano Freddi, Helmut Schwab, and Georg M. Guebitz. 2012. “Characterization of a New Cutinase from Thermobifida alba for PET-Surface Hydrolysis.” Biocatalysis and Biotransformation. https://doi.org/10.3109/10242422.2012.644435.
Ribitsch, Doris, Antonio Orcal Yebra, Sabine Zitzenbacher, Jing Wu, Susanne Nowitsch, Georg Steinkellner, Katrin Greimel, et al. 2013. “Fusion of Binding Domains to Thermobifida cellulosilytica Cutinase to Tune Sorption Characteristics and Enhancing PET Hydrolysis.” Biomacromolecules 14 (6): 1769-76.
Roth, Christian, Ren Wei, Thorsten Oeser, Johannes Then, Christina Föllner, Wolfgang Zimmermann, and Norbert Striter. 2014. “Structural and Functional Studies on a Thermostable Polyethylene Terephthalate Degrading Hydrolase from Thermobifida fusca.” Applied Microbiology and Biotechnology 98 (18): 7815-23.
Shannon, Paul, Andrew Markiel, Owen Ozier, Nitin S. Baliga, Jonathan T. Wang, Daniel Ramage, Nada Amin, Benno Schwikowski, and Trey Ideker. 2003. “Cytoscape: A Software Environment for Integrated Models of Biomolecular Interaction Networks.” Genome Research 13 (11): 2498-2504.
Shi, Lixia, Haifeng Liu, Songfeng Gao, Yunxuan Weng, and Leilei Zhu. 2021. “Enhanced Extracellular Production of IsPETase in Escherichia coli via Engineering of the pelB Signal Peptide.” Journal of Agricultural and Food Chemistry. https://doi.org/10.1021/acs.jafc.0c07469.
Shimodaira, Hidetoshi. 2002. “An Approximately Unbiased Test of Phylogenetic Tree Selection.” Systematic Biology 51 (3): 492-508.
Shirke, Abhijit N., Christine White, Jacob A. Englaender, Allison Zwarycz, Glenn L. Butterfoss, Robert J. Linhardt, and Richard A. Gross. 2018. “Stabilizing Leaf and Branch Compost Cutinase (LCC) with Glycosylation: Mechanism and Effect on PET Hydrolysis.” Biochemistry. https://doi.org/10.1021/acs.biochem.7b01189.
Silva, Carla, Shi Da, Nidia Silva, Teresa Matami, Rita Aranjo, Madalena Martins, Sheng Chen, et al. 2011. “Engineered Thermobifida fusca Cutinase with Increased Activity on Polyester Substrates.” Biotechnology Journal 6 (10): 1230-39.
Son, Hyeoncheol Francis, In Jin Cho, Seongjoon Joo, Hogyun Seo, Hye-Young Sagong, So Young Choi, Sang Yup Lee, and Kyung-Jin Kim. 2019. “Rational Protein Engineering of Thermo-Stable PETase from Ideonella sakaiensis for Highly Efficient PET Degradation.” ACS Catalysis. https://doi.org/10.1021/acscatal.9b00568.
Son, Hyeoncheol Francis, Seongjoon Joo, Hogyun Seo, Hye-Young Sagong, Seul Hoo Lee, Hwaseok Hong, and Kyung-Jin Kim. 2020. “Structural Bioinformatics-Based Protein Engineering of Thermo-Stable PETase from Ideonella sakaiensis.” Enzyme and Microbial Technology 141 (November): 109656.
Spence, Matthew A., Joe A. Kaczmarski, Jake W. Saunders, and Colin J. Jackson. 2021. “Ancestral Sequence Reconstruction for Protein Engineers.” Current Opinion in Structural Biology 69 (May): 131-41.
Sulaiman, Sintawee, Saya Yamato, Eiko Kanaya, Joong-Jae Kim, Yuichi Koga, Kazufumi Takano, and Shigenori Kanaya. 2012. “Isolation of a Novel Cutinase Homolog with Polyethylene Terephthalate-Degrading Activity from Leaf-Branch Compost by Using a Metagenomic Approach.” Applied and Environmental Microbiology 78 (5): 1556.
Sweigard, J. A., F. G. Chumley, and B. Valent. 1992. “Cloning and Analysis of CUT1, a Cutinase Gene from Magnaporthe grisea.” Molecular & General Genetics: MGG 232 (2): 174-82.
Then, Johannes, Ren Wei, Thorsten Oeser, Andre Gerdts, Juliane Schmidt, Markus Barth, and Wolfgang Zimmermann. 2016. “A Disulfide Bridge in the Calcium Binding Site of a Polyester Hydrolase Increases Its Thermal Stability and Activity against Polyethylene Terephthalate.” FEBS Open Bio 6 (5): 425-32.
Thomas, Adam, Rhys Cutlan, William Finnigan, Mark van der Giezen, and Nicholas Harmer. 2019. “Highly Thermostable Carboxylic Acid Reductases Generated by Ancestral Sequence Reconstruction.” Communications Biology 2 (November): 429.
Tournier, V., C. M. Topham, A. Gilles, B. David, C. Folgoas, E. Moya-Leclair, E. Kamionka, et al. 2020. “An Engineered PET Depolymerase to Break down and Recycle Plastic Bottles.” Nature 580 (7802): 216-19.
Wei, Ren, Thorsten Oeser, Juliane Schmidt, Ren6 Meier, Markus Barth, Johannes Then, and Wolfgang Zimmermann. 2016. “Engineered Bacterial Polyester Hydrolases Efficiently Degrade Polyethylene Terephthalate due to Relieved Product Inhibition.” Biotechnology and Bioengineering 113 (8): 1658-65.
Wheeler, Lucas C., and Michael J. Harms. 2021. “Were Ancestral Proteins Less Specific?” Molecular Biology and Evolution 38 (6): 2227-39.
Whelan, S., and N. Goldman. 2001. “A General Empirical Model of Protein Evolution Derived from Multiple Protein Families Using a Maximum-Likelihood Approach.” Molecular Biology and Evolution 18 (5): 691-99.
Wilding, Matthew, Thomas S. Peat, Subha Kalyaanamoorthy, Janet Newman, Colin Scott, and Lars S. Jermiin. 2017. “Reverse Engineering: Transaminase Biocatalyst Development Using Ancestral Sequence Reconstruction.” Green Chemistry. https://doi.org/10.1039/c7gc02343j.
Yang, Ziheng. 2007. “PAML 4: Phylogenetic Analysis by Maximum Likelihood.” Molecular Biology and Evolution 24 (8): 1586-91.
Yoshida, Shosuke, Kazumi Hiraga, Toshihiko Takehana, Ikuo Taniguchi, Hironao Yamaji, Yasuhito Maeda, Kiyotsuna Toyohara, Kenji Miyamoto, Yoshiharu Kimura, and Kohei Oda. 2016. “A Bacterium That Degrades and Assimilates Poly(ethylene Terephthalate).” Science 351 (6278): 1196-99.

1. A polypeptide having esterase activity, wherein the esterase activity is capable of converting a
a. monoester terephthalate to terephthalic acid and an alcohol;
b. diester terephthalate to a monoester terephthalate and an alcohol; or
c. diester terephthalate to terephthalic acid and an alcohol;
wherein the polypeptide comprises an amino acid sequence selected from the group consisting of:
i. amino acids 5-261 of SEQ ID NO:2 or an amino acid sequence having at least 85% sequence identity thereto;
ii. amino acids 5-261 of SEQ ID NO:3 or an amino acid sequence having at least 77% sequence identity thereto;
iii. amino acids 5-261 of SEQ ID NO: 4 or an amino acid sequence having at least 75% sequence identity thereto;
iv. amino acids 5-261 of SEQ ID NO: 5 or an amino acid sequence having at least 95% sequence identity thereto; and
v. amino acids 5-261 of SEQ ID NO: 6 or an amino acid sequence having at least 96% sequence identity thereto,
wherein the polypeptide is not SEQ ID NO:1 or SEQ ID NO: 12.

2-13. (canceled)

14. The polypeptide of claim 1, wherein the polypeptide comprises the amino acid sequence of SEQ ID NO:119.

15. A method for the enzymatic hydrolysis of monoester terephthalate and/or diester terephthalate generated as by-product of degradation of PET,
wherein
a. the monoester terephthalate is a mono-C₁-C₁₀ alkyl terephthalate, optionally substituted with benzyl, and wherein the mono-ester terephthalate is not mono-(2-hydroxyethyl)terephthalate (MHET);
b. the diester terephthalate is a di-C₁-C₁₀ alkyl terephthalate, optionally substituted with benzyl;
the method comprising exposing the monoester terephthalate and/or diester terephthalate to a polypeptide having esterase activity, under conditions sufficient to enable the polypeptide to convert the:
i. monoester terephthalate to terephthalic acid and an alcohol;
ii. diester terephthalate to a monoester terephthalate and an alcohol; or
iii. diester terephthalate to terephthalic acid and an alcohol.

16. The method of claim 15, wherein the polypeptide comprises the amino acid sequence of amino acids 5-261 of SEQ ID NO:1 or an amino acid sequence having at least 70% sequence identity thereto.

17. The method of claim 15, wherein the monoester terephthalate is a mono-C₆-C₁₀ alkyl terephthalate, optionally substituted with benzyl.

18. The method of claim 17, wherein the monoester terephthalate is selected from a group consisting of monobenzyl terephthalate (MBZT), monohexyl terephthalate, monoheptyl terephthalate (MHPT) and monooctyl terephthalate (MOCT).

19. (canceled)

20. The method of claim 17, wherein the polypeptide comprises the amino acid sequence of amino acids 5-261 of any one of SEQ ID NOs: 9-11 or an amino acid sequence that has at least 70% sequence identity to any of the foregoing.

21. The method of claim 17, wherein the polypeptide comprises the amino acid sequence of SEQ ID NO: 119.

22. The method of claim 15, wherein the diester terephthalate is a di-C₆-C₁₀ alkyl terephthalate, optionally substituted with benzyl.

23. The method of claim 22, wherein the diester terephthalate is selected from a group consisting of dibenzyl terephthalate (DBZT), dihexyl terephthalate (DHXT), diheptyl terephthalate (DHPT) and dioctyl terephthalate (DOCT).

24. The method of claim 23, wherein the diester terephthalate is DBZT or DOCT.

25. The method of claim 22, wherein the polypeptide comprises the amino acid sequence of amino acids 5-261 of any one of SEQ ID NOs: 6-8 or an amino acid sequence having at least 70% sequence identity to any of the foregoing.

26. The method of claim 15, wherein the monoester terephthalate and/or diester terephthalate is generated by a process comprising:
a. exposing PET to sodium hydroxide, and/or
b. contacting the PET to an esterase.

27. The method of claim 26, wherein monoester terephthalate and/or diester terephthalate is generated by a process comprising:
c. subjecting the PET to base-catalysed transesterification with a C₁-C₁₀ mono-alcohol; and/or
d. contacting the PET to an esterase.

28. The method of claim 27, wherein the C₁-C₁₀ mono-alcohol is a C₆-C₁₀ mono-alcohol.

29. The method of claim 28, wherein the C₆-C₁₀ mono-alcohol is a benzyl alcohol, an octanol or a heptanol.

30. The method of claim 29, wherein the C₆-C₁₀ mono-alcohol is 1-octanol.

31. The method of claim 15, further comprising recovering the terephthalic acid and/or the alcohol.

32. A composition comprising the terephthalic acid and/or alcohol recovered by the method of claim 31.

33. A composition comprising the polypeptide of claim 1.

34. A polynucleotide comprising a nucleic acid sequence encoding the polypeptide of claim 1.

35-36. (canceled)

37. A host cell genetically modified to express the polypeptide of claim 1.

38. (canceled)

39. A method of recycling a plastic product comprising PET, the method comprising
a. degrading the PET by base-catalysed transesterification to generate monoester terephthalate and/or diester terephthalate, and
b. contacting the monoester terephthalate and/or diester terephthalate to the polypeptide of claim 1, under conditions sufficient to enable the polypeptide to convert the:
i. monoester terephthalate to terephthalic acid and an alcohol;
ii. diester terephthalate to a monoester terephthalate and an alcohol; or
iii. diester terephthalate to terephthalic acid and an alcohol.

40. The method of claim 39, wherein degrading the PET further comprises simultaneously or sequentially exposing the PET to a PETase and/or a cutinase having PETase activity.

41. (canceled)

42. A polypeptide having esterase activity, wherein the esterase activity is capable of converting a
a. monoester terephthalate to terephthalic acid and an alcohol;
b. diester terephthalate to a monoester terephthalate and an alcohol; or
c. diester terephthalate to terephthalic acid and an alcohol;
wherein the polypeptide comprises an amino acid sequence of SEQ ID NO:119.


SEQUENCE LISTING (124 sequences)

SEQ ID NO: 1 - protein, 261 aa, synthetic construct
MAANPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 2 - protein, 261 aa, synthetic construct
MAENPYERGPDPTESSIEALRGPFAVSEESVSRLSVSGFGGGTIYYPTDTSEGTFGAVAISPGYTGTQSSMAWLGPRIASQGFVVFTIDTNTTYDQPDSRARQLQAALDYLVEDSSVRDRIDPNRLGVMGHSMGGGGTLRAAEDRPDLKAAIPLTPWHLNKNWSSVRVPTLIIGAENDTIAPVRTHAEPFYESLPSSLDKAYLELDGASHFAPNISNTTIAKYSISWLKRFVDDDTRYEQFLCPPPRTGSEISEYRSTCPF

SEQ ID NO: 3 - protein, 261 aa, synthetic construct
MAENPYERGPDPTEASIEAERGPFAIAQVSVPAGSGSGFGGGTIYYPTDTSQGTFGAVAISPGFTATEASIAWLGPRLASQGFVVITIDTNSRYDQPSARADQLLAALDYLTQSSSVRSRIDPNRLAVMGHSMGGGGTLEAAENRPSLKAAIPLAPWNTNKNWSSVRVPTMIIGAQNDTIAPVGSHAEPFYNSLPASPEKAYLELNGADHFAPTSSNTTIAKYSISWLKRFVDDDTRYDQFLCPAPSPDSAISEYRSTCPH

SEQ ID NO: 4 - protein, 261 aa, synthetic construct
MQANPYQRGPDPTSASLEASSGPFSVSTTSVSRLSASGFGGGTIYYPTTTSSGKYGAVAISPGYTATQSSIAWLGRRLASHGFVVITIDTNTTLDQPDSRATQLMAALNYVVNSSTVRSRVDASRLAVMGHSMGGGGTLIAAENNPSLKAAIPLTPWHTSKNFSSVRVPTLIIGAENDTIAPVSSHAKPFYNSLPSTTSKAYLELNGASHFAPNSSNTPIGKYSISWMKRFVDNDTRYSPFLCGAPHQGAVISEYRDNCPY

SEQ ID NO: 5 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELDGATHFAPNIPNKIIGKYSVAWLKRFVDNDTRYTQFLCPGPRDGGEVEEYRSTCPF

SEQ ID NO: 6 - protein, 261 aa, synthetic construct; identical to SEQ ID NO: 5

SEQ ID NO: 7 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALNYMINRSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHFAPNIPNKIIGKYSVAWLKRFVDNDTRYTQFLCPGPRDGGEVEEYRSTCPF

SEQ ID NO: 8 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALNYMINRSTVRSRIDSSRLAVMGHSMGGGGTLRLASQRPDLKAAIPLTPWHLNKNWSSVRVPTLIIGADLDTIAPVATHAKPFYNSLPSSISKAYLELDGATHFAPNIPNKIIGKYSVAWLKRFVDNDTRYTQFLCPGPRDGGEVEEYRSTCPF

SEQ ID NO: 9 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRAEQLNAALNYMINRSTVRSRIDSSRLAVMGHSMGGGGTLRLASQRPDLKAAIPLTPWHLNKNWSSVRVPTLIIGADLDTIAPVATHAKPFYNSLPSSISKAYLELDGATHFAPNIPNKIIGKYSVAWLKRFVDNDTRYTQFLCPGPRDGGEVEEYRSTCPF

SEQ ID NO: 10 - protein, 261 aa, synthetic construct
MAANPYERGPNPTDALLEARSGPFSVSEENVSRLSASGFGGGTIYYPRENSSNTYGAVAISPGYTGTEASIAWLGERIASHGFVVITIDTITTLDQPDSRAEQLNAALNHMINRSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHFAPNIPNKIIGKYSVAWLKRFVDNDTRYTQFLCPGPRDGGEVEEYRSTCPF

SEQ ID NO: 11 - protein, 261 aa, synthetic construct
MAANPYERGPNPTDALLEARSGPFSVSEENVSRLSASGFGGGTIYYPRENSENTYGAVAISPGYTGTEASIAWLGERIASHGFVVITIDTITTLDQPDSRAEQLNAALNHMINRSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHFAPNIPNKIIGKYSVAWLKRFVDNDTRYTQFLCPGPRDGGEVEEYRSTCPF

SEQ ID NO: 12 - protein, 261 aa, synthetic construct
MAANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 13 - protein, 261 aa, synthetic construct
MAANPYERGPDPTESSLEASSGPFSVSQTSVSRLSVSGFGGGTIYYPTDTSSGTYGAVAISPGYTATQSSIAWLGPRLASHGFVVITIDTNSTFDQPDSRARQLMAALDYLVNRSPVRSRIDSSRLAVMGHSMGGGGTLRAAEDNPSLKAAIPLTPWHTDKNWSSVRVPTLIIGAENDTIAPVSSHAKPFYNSLPSSTSKAYLELNGASHFAPNSSNTTIGKYSVSWLKRFVDNDTRYSQFLCPAPHDDSAISEYRSTCPY

SEQ ID NO: 14 - protein, 261 aa, synthetic construct
MADNPYERGPAPTESSIEASRGPFAVSQTSVSSLSVSGFGGGTIYYPTSTSEGTFGAVAISPGYTATQSSIAWLGPRLASQGFVVFTIDTNTRYDQPDSRGRQLLAALDYLTQRSSVRSRIDASRLGVMGHSMGGGGTLEAAEDRPSLQAAIPLTPWNLDKNWSSVRVPTMIIGAENDTIAPVSSHSEPFYTSLPSSLDKAYLELNGASHFAPNTSNTTIAKYSISWLKRFIDNDTRYEQFLCPAPSRDTTISEYRDTCPH

SEQ ID NO: 15 - protein, 261 aa, synthetic construct
MADNPYERGPDPTESSIEASRGPFAVSQTSVSRLSVSGFGGGTIYYPTSTSEGTFGAVAISPGYTATQSSIAWLGPRLASQGFVVFTIDTNTRYDQPDSRGDQLLAALDYLTQRSSVRSRIDPSRLAVMGHSMGGGGTLEAAKDRPSLKAAIPLTPWNTDKNWSSVTVPTLIIGAENDTIAPVSSHSKPFYNSLPSSPEKAYLELNGASHFAPNSSNTTIAKYSIAWLKRFVDNDTRYSQFLCPAPSADSAISEYRDTCPY

SEQ ID NO: 16 - protein, 261 aa, synthetic construct
MQANPYQRGPDPTSSSLEASSGPFSVSTTSVSRLSVSGFGGGTIYYPTNTSSGTYGAVAISPGYTATQSSIAWLGPRLASHGFVVITIDTNSTFDQPDSRATQLMAALDYVVNSSPVRSRVDSSRLAVMGHSMGGGGTLRAAEDNPSLKAAIPLTPWHTDKNFSSVRVPTLIIGAENDTVAPVSSHAKPFYNSLPSSTPKAYLELNGASHFAPNSSNTTIGKYSVSWMKRFVDNDTRYSQFLCPAPHDDSAISEYRSTCPY

SEQ ID NO: 17 - protein, 261 aa, synthetic construct
MQANPYQRGPDPTESSLEASSGPFSVSTTSVSRLSVSGFGGGTIYYPTSTSSGTYGAVAISPGYTATQSSIAWLGPRLASHGFVVITIDTNSTFDQPDSRATQLMAALNYLVNSSSVRSRIDSSRLAVMGHSMGGGGTLRAAEDNPSLKAAIPLTPWHTDKNFSSVTVPTLIIGAENDTIAPVSSHAKPFYNSLPSSTSKAYVELNGASHFAPNSSNTTIGKYSVSWMKRFVDNDTRYSQFLCGAPHDDSAISEYRSNCPY

SEQ ID NO: 18 - protein, 261 aa, synthetic construct
MQANPYQRGPDPTSASLEASSGPFSVSTTSVSRLSASGFGGGTIYYPTTTSSGTYGAVAISPGYTATQSSIAWLGPRLASHGFVVITIDTNSTFDQPDSRATQLMAALNYVVNSSTVRSRVDSSRLAVMGHSMGGGGTLQAAEDNPSLKAAIPLTPWHTNKNFSSVRVPTLIIGAENDTIAPVSSHAKPFYNSLPSSTSKAYVELNGASHFAPNSSNTTIGKYSVSWMKRFVDNDTRYSPFLCGAPHDDSAISEYRSTCPY

SEQ ID NO: 19 - protein, 261 aa, synthetic construct
MAANPYERGPDPTNASLEASSGPFSVSETSVSRLSASGFGGGTIYYPTSTSEGTYGAVAISPGYTGTQSSIAWLGPRLASHGFVVITIDTNTTLDQPDSRASQLMAALNYLVNRSTVRSRIDASRLAVMGHSMGGGGTLRAAEQRPSLKAAIPLTPWHTDKNWSSVRVPTLIIGAENDTIAPVSSHAKPFYNSLPSTISKAYLELNGASHFAPNSSNTTIGKYSISWLKRFVDNDTRYSQFLCPAPRQGALIEEYRDTCPY

SEQ ID NO: 20 - protein, 261 aa, synthetic construct
MQANPYQRGPDPTSSSLEASSGPFSVSTTSVSSLSVSGFGGGTIYYPTNTSSGTYGAVAISPGYTATQSSIAWLGPRLASHGFVVITIDTNSTFDQPDSRARQLMAALNYLVNRSPVRSRVDSSRLAVMGHSMGGGGTLRAAEDNPSLKAAIPLTPWHTDKNFSSVRVPTLIIGAENDTIAPVSSHAKPFYNSLPSSTSKAYLELNGASHFAPNSSNTTIGKYSVSWMKRFVDNDTRYSQFLCPAPHDDSAISEYRDTCPY

SEQ ID NO: 21 - protein, 261 aa, synthetic construct
MAANPYERGPDPTESSLEASSGPFSVSTTSVSRLSVSGFGGGTIYYPTSTSEGTYGAVAISPGYTATQSSIAWLGRRLASHGFVVITIDTNTTFDQPDSRADQLMAALNYLVNRSSVRSRIDSSRLAVMGHSMGGGGTLRAAKDRPSLKAAIPLTPWHTDKNWSSVTVPTLIIGAENDTIAPVSSHAKPFYNSLPSSTSKAYLELNGASHFAPNSSNTTIGKYSVAWLKRFVDNDTRYSQFLCPAPHDGSAISEYRDTCPY

SEQ ID NO: 22 - protein, 261 aa, synthetic construct
MQANPYQRGPDPTSASLEASSGPFSVSTTSVSRLSASGFGGGTIYYPTSTSSGTYGAVAISPGYTATQSSIAWLGRRLASHGFVVITIDTNTTLDQPDSRATQLMAALNYLVNRSTVRSRIDASRLAVMGHSMGGGGTLRAAEQNPSLKAAIPLTPWHTDKNFSSVRVPTLIIGAENDTIAPVSSHAKPFYNSLPSTTSKAYLELNGASHFAPNSSNTPIGKYSISWMKRFVDNDTRYSQFLCGAPHQGAVISEYRDNCPY

SEQ ID NO: 23 - protein, 261 aa, synthetic construct
MAENPYERGPDPTESSIEATRGPFAVSQTSVSRLSVSGFGGGTIYYPTSTSEGTFGAVAISPGYTATQSSIAWLGPRLASQGFVVITIDTNSRYDQPASRGDQLLAALDYLTQSSSVRSRIDPSRLAVMGHSMGGGGTLEAAKDRPSLKAAIPLTPWNTDKNWSEVRVPTLIIGAENDTVAPVSSHAEPFYNSLPSSPEKAYLELNGASHFAPNSSNTTIAKYSISWLKRFVDDDTRYDQFLCPAPSPDSAISEYRSTCPH

SEQ ID NO: 24 - protein, 261 aa, synthetic construct
MAANPYERGPDPTEASLEASSGPFSVSQTSVSSLSVSGFGGGTIYYPTSTSSGTYGAVAISPGYTATQSSIAWLGPRLASHGFVVITIDTNSTFDQPDSRARQLMAALDYLVNRSSVRSRIDSSRLAVMGHSMGGGGTLRAAEDRPSLKAAIPLTPWHTNKNWSSVRVPTLIIGAENDTIAPVSSHAKPFYNSLPSSTSKAYLELNGASHFAPNSSNTTIGKYSVSWLKRFVDNDTRYSQFLCPAPHDDSAISEYRDTCPY

SEQ ID NO: 25 - protein, 261 aa, synthetic construct
MAANPYERGPDPTEASLEASSGPFSVSETSVSRLSASGFGGGTIYYPTTTSEGTYGAVAISPGYTATQSSIAWLGPRLASHGFVVITIDTNSTFDQPDSRADQLMAALNYLVNRSSVRSRIDSSRLAVMGHSMGGGGTLQAAEDRPSLKAAIPLTPWHTNKNWSSVRVPTLIIGAENDTIAPVSSHAKPFYNSLPSSTSKAYLELNGASHFAPNSSNTTIGKYSVSWLKRFVDNDTRYSQFLCPAPHDDSAISEYRSTCPY

SEQ ID NO: 26 - protein, 261 aa, synthetic construct
MQANPYQRGPDPTSSSLEASSGPFSVSTTSVSRLSASGFGGGTIYYPTTTSSGTYGAVAISPGYTATQSSIAWLGRRLASHGFVVITIDTNSTFDQPDSRATQLMAALNYLVNSSTVRSRIDASRLAVMGHSMGGGGTLRAAEDNPSLKAAIPLTPWHTDKNFSSVRVPTLIIGAENDTIAPVSTHAKPFYNSLPSSTSKAYVELNGASHFAPNSSNTPIGKYSISWMKRFVDNDTRYSQFLCGAPHDGSAISEYRDNCPY

SEQ ID NO: 27 - protein, 261 aa, synthetic construct
MADNPYERGPAPTEASIEASRGPFAISQVSVPSASGSGFGGGTIYYPTDTSQGTFGAVAISPGFTATEASIAWLGPRLASQGFVVITIDTNSRYDQPDSRGKQLLAALDYLTQKSSVRSRIDPNRLAVMGHSMGGGGTLEAAENRPSLKAAIPLTPWHTDKNWSSVRVPTMIIGAENDTIAPVGSHAEPFYNSLPSSPEKAYLELKGADHFAPTSSNTTIAKYSISWLKRFVDDDTRYDQFLCPAPSSDSAISEYRDTCPH

SEQ ID NO: 28 - protein, 261 aa, synthetic construct
MAENPYERGPAPTESSIEATRGPFAVSQTSVSSLSVSGFGGGTIYYPTSTSEGTFGAVAISPGYTASQSSIAWLGPRLASQGFVVFTIDTNTRYDQPASRGDQLLAALDYLTQRSSVRSRIDASRLGVMGHSMGGGGTLEAAKDRPSLQAAIPLTGWNLDKNWSEVRVPTLVIGAENDTIAPVSSHSEPFYNSLPSSLDKAYLELNGASHFAPNSSNTTIAKYSISWLKRFIDNDTRYEQFLCPAPRPGSTIEEYRDTCPH

SEQ ID NO: 29 - protein, 261 aa, synthetic construct
MAANPYERGPDPTESSLEASSGPFSVSQTSVSRLSVSGFGGGTIYYPTSTSSGTYGAVAISPGYTATQSSIAWLGPRLASHGFVVITIDTNSTFDQPDSRADQLMAALNYLVNRSSVRSRIDSSRLAVMGHSMGGGGTLRAAEDRPSLKAAIPLTPWHTDKNWSSVTVPTLIIGAENDTIAPVSSHAKPFYNSLPSSTSKAYLELNGASHFAPNSSNTTIGKYSISWLKRFVDNDTRYSQFLCPAPHDDSAISEYRSTCPY

SEQ ID NO: 30 - protein, 261 aa, synthetic construct
MAANPYERGPDPTESSLEASSGPFSVSETSVSRLSVSGFGGGTIYYPTSTSSGTYGAVAISPGYTATQSSIAWLGPRLASHGFVVITIDTNSTFDQPDSRARQLMAALDYLVNSSSVRSRIDSSRLAVMGHSMGGGGTLRAAEDRPSLKAAIPLTPWHTNKNWSSVRVPTLIIGAENDTVAPVSSHAKPFYNSLPSSTPKAYLELNGASHFAPNSSNTTIGKYSVAWLKRFVDNDTRYSQFLCPAPHDDSAISEYRSTCPY

SEQ ID NO: 31 - protein, 261 aa, synthetic construct
MAANPYERGPDPTESSLEASSGPFSVSETSVSRLSVSGFGGGTIYYPTDTSEGTYGAVAISPGYTATQSSIAWLGPRLASHGFVVITIDTNSTFDQPDSRARQLQAALNYLVNSSSVRSRIDSNRLAVMGHSMGGGGTLRAAEDNPSLKAAIPLTPWHTNKNWSSVTVPTLIIGAENDTIAPVSSHAKPFYNSLPSSTSKAYLELNGASHFAPNSSNTTIGKYSISWLKRFVDNDTRYSQFLCPAPHDDSAISEYRSTCPY

SEQ ID NO: 32 - protein, 261 aa, synthetic construct
MQANPYQRGPDPTSSSLEASSGPFSVSTTSVSRLSVSGFGGGTIYYPTTTSSGTYGAVAISPGYTATQSSIAWLGPRLASHGFVVITIDTNSTFDQPDSRATQLMAALNYVVNSSPVRSRVDSSRLAVMGHSMGGGGTLRAAEDNPSLKAAIPLTPWHTNKNFSSVRVPTLIIGAENDTVAPVSSHAKPFYNSLPSSTPKAYLELNGASHFAPNSSNTTIGKYSVAWMKRFVDNDTRYSPFLCGAPHDDSAISEYRSTCPY

SEQ ID NO: 33 - protein, 261 aa, synthetic construct
MAANPYERGPDPTEASLEASSGPFSVSTTSVSSLSVSGFGGGTIYYPTSTSEGTYGAVAISPGYTATQSSIAWLGRRLASHGFVVITIDTNSTFDQPDSRADQLMAALNYLVNRSSVRSRIDSSRLAVMGHSMGGGGTLRAAKDRPSLKAAIPLTPWHTDKNWSSVRVPTLIIGAENDTIAPVSSHAKPFYNSLPSSTSKAYLELNGASHFAPNSSNTTIGKYSVSWLKRFVDNDTRYSQFLCPAPHDDSAISEYRDTCPY

SEQ ID NO: 34 - protein, 261 aa, synthetic construct
MAENPYERGPAPTESSIEASRGPFAVSQTSVSSLSVSGFGGGTIYYPTSTSEGTFGAVAISPGYTATQSSIAWLGPRLASQGFVVFTIDTNTRYDQPASRGDQLLAALDYLTQRSSVRSRIDASRLGVMGHSMGGGGTLEAAKDRPSLQAAIPLTPWNLDKNWSEVRVPTLIIGAENDTIAPVSSHSEPFYNSLPSSLDKAYLELNGASHFAPNSSNTTIAKYSISWLKRFIDNDTRYEQFLCPAPSPDSTISEYRDTCPH

SEQ ID NO: 35 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRAEQLNAALNYMINRSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHFAPNIPNKIIGKYSVAWLKRFVDNDTRYTQFLCPGPRDGGEVEEYRSTCPF

SEQ ID NO: 36 - protein, 261 aa, synthetic construct
MAANPYERGPDPTESSLEASSGPFSVSETSVSRLSVSGFGGGTIYYPTSTSSGTYGAVAISPGYTATQSSIAWLGPRLASHGFVVITIDTNTTFDQPDSRARQLMAALNYLVNSSSVRSRIDSSRLAVMGHSMGGGGTLRAAEDNPSLKAAIPLTPWHTDKNWSSVRVPTLIIGAENDTIAPVSSHAKPFYNSLPSSTPKAYLELNGASHFAPNSSNTTIGKYSISWLKRFVDNDTRYSQFLCPAPHDDSAISEYRSTCPY

SEQ ID NO: 37 - protein, 261 aa, synthetic construct
MAANPYERGPDPTEASLEATSGPFSVSETSVSRLSVSGFGGGTIYYPTDTSSGTYGAVAISPGYTATQSSIAWLGPRLASHGFVVITIDTNSTFDQPDSRARQLMAALNYLVNSSPVRSRIDSSRLAVMGHSMGGGGTLRAAEDNPSLKAAIPLTPWHTNKNWSSVRVPTLIIGAENDTIAPVSSHAKPFYNSLPSSTSKAYLELNGASHFAPNSSNTTIGKYSISWLKRFVDNDTRYSQFLCPAPHDGSAISEYRSTCPY

SEQ ID NO: 38 - protein, 261 aa, synthetic construct
MAANPYERGPNPTDALLEARSGPFSVSEENVSRLSASGFGGGTIYYPRENSDNTYGAVAISPGYTGTEASIAWLGERIASHGFVVITIDTITTLDQPDSRAEQLNAALNHMINRSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHFAPNIPNKIIGKYSVAWLKRFVDNDTRYTQFLCPGPRDGGEVEEYRSTCPF

SEQ ID NO: 39 - protein, 261 aa, synthetic construct
MAANPYERGPNPTDALLEARSGPFSVSEERASRLGADGFGGGTIYYPRENSDNTYGAVAISPGYTGTQASVAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVLTHAKPFYNSLPTSISKAYLELDGATHFAPNIPNKIIGKYSVAWLKRFVDNDTRYTQFLCPGPRDGGEVEEYRSTCPF

SEQ ID NO: 40 - protein, 261 aa, synthetic construct
MAANPYERGPNPTDALLEARSGPFSVSEENVSRLSASGFGGGTIYYPRENSSNTYGAVAISPGYTGTQASIAWLGERIASHGFVVITIDTNTTLDQPDSRAEQLNAALDYMINRSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHFAPNIPNKIIGKYSVAWLKRFVDNDTRYTQFLCPGPRDGGEVEEYRSTCPF

SEQ ID NO: 41 - protein, 261 aa, synthetic construct
MAANPYERGPNPTDALLEARSGPFSVSEENVSRLSASGFGGGTIYYPRENSSNTYGAVAISPGYTGTQASIAWLGERIASHGFVVITIDTNTTLDQPDSRARQLNAALNYMINRSTVRSRIDSSRLAVMGHSMGGGGTLRLASQRPDLKAAIPLTPWHLNKNWSSVRVPTLIIGADLDTIAPVATHAKPFYNSLPSSISKAYLELDGATHFAPNIPNKIIGKYSVAWLKRFVDNDTRYTQFLCPGPRDGGEVEEYRSTCPF

SEQ ID NO: 42 - protein, 261 aa, synthetic construct
MAANPYERGPDPTESSLEASSGPFSVSETSVSRLSASGFGGGTIYYPTTTSEGTYGAVAISPGYTGTQSSIAWLGPRLASHGFVVITIDTNTTFDQPDSRARQLMAALNYLVNRSSVRSRIDASRLAVMGHSMGGGGTLRAAEQRPSLKAAIPLTPWHTDKNWSSVRVPTLIIGAENDTIAPVSTHAKPFYNSLPSSISKAYLELNGASHFAPNTSNTTIGKYSISWLKRFVDNDTRYTQFLCPAPRDGSAIEEYRDTCPY

SEQ ID NO: 43 - protein, 261 aa, synthetic construct
MAANPYERGPNPTDALLEARSGPFSVSEENVSRLSASGFGGGTIYYPRENSDNTYGAVAISPGYTGTQASIAWLGERIASHGFVVITIDTNTTLDQPDSRARQLNAALNYMINRSTVRSRIDSSRLAVMGHSMGGGGTLRLASQRPDLKAAIPLTPWHLNKNWSSVRVPTLIIGADLDTIAPVATHAKPFYNSLPSSISKAYLELDGATHFAPNIPNKIIGKYSVAWLKRFVDNDTRYTQFLCPGPRDGGEVEEYRSTCPF

SEQ ID NO: 44 - protein, 261 aa, synthetic construct
MAENPYERGPAPTESSIEASSGPFSVSTTSVSSLSVSGFGGGTIYYPTSTSEGTFGAVAISPGYTATQSSIAWLGRRLASHGFVVITIDTNSTYDQPDSRGDQLLAALDYLTQRSSVRSRIDPSRLAVMGHSMGGGGTLEAAKDRPSLKAAIPLTPWHTDKNWSSVRVPTLIIGAENDTIAPVSSHAIPFYNSLPSSTEKAYLELNGASHFAPNSSNTTIGKYSVSWLKRFVDNDTRYSQFLCPAPSPDSAISEYRDTCPH

SEQ ID NO: 45 - protein, 261 aa, synthetic construct
MADNPYERGPDPTESSIEASRGPFAVSETSVSRLSVSGFGGGTIYYPTTTSEGTFGAVAISPGYTGTQSSIAWLGPRLASQGFVVITIDTNTTYDQPDSRARQLLAALDYLTQRSSVRSRIDASRLAVMGHSMGGGGTLRAAEDRPSLKAAIPLTPWHTDKNWSSVRVPTLIIGAENDTIAPVSSHAEPFYNSLPSSLPKAYLELNGASHFAPNTSNTTIAKYSISWLKRFVDNDTRYTQFLCPAPRPGSAIEEYRDTCPY

SEQ ID NO: 46 - protein, 261 aa, synthetic construct
MADNPYERGPAPTESSIEASRGPFAVSQTSVSSLSVSGFGGGTIYYPTSTSEGTFGAVAISPGYTATQSSIAWLGPRLASQGFVVFTIDTNTRYDQPDSRGRQLLAALDYLTQRSSVRSRIDPSRLGVMGHSMGGGGTLEAAKDRPSLQAAIPLTPWNLDKNWSSVRVPTLIIGAENDTIAPVASHSEPFYNSLPSSLDKAYLELNGASHFAPNSSNTTIAKYSISWLKRFIDNDTRYEQFLCPAPSRDSTISEYRDTCPH

SEQ ID NO: 47 - protein, 261 aa, synthetic construct
MQANPYQRGPDPTSSSLEASSGPFSVSTTSVSRLSVSGFGGGTIYYPTNTSSGTYGAVAISPGYTATQSSIAWLGPRLASHGFVVITIDTNSTFDQPDSRATQLMAALNYVVNSSPVRSRVDSNRLAVMGHSMGGGGTLRAAEDNPSLKAAIPLTPWHTNKNFSSVRVPTLIIGAENDTIAPVSSHAKPFYNSLPSSTSKAYLELNGASHFAPNSSNTTIGKYSVSWMKRFVDNDTRYSQFLCPAPHDDSAISEYRSTCPY

SEQ ID NO: 48 - protein, 261 aa, synthetic construct
MAENPYERGPDPTESSIEASRGPFAVSQTSVSSLSVSGFGGGTIYYPTSTSEGTFGAVAISPGYTATQSSIAWLGPRLASQGFVVFTIDTNTRYDQPASRGRQLLAALDYLTQRSSVRSRIDPSRLAVAGHSMGGGGTLEAAEDRPSLQAAIPLAPWNLDKNWSSVRVPTLIIGGESDTVAPVSSHSEPFYNSLPSSPDKAYLELNNASHFFPNTSNTTMAKYMISWLKRFVDNDTRYEQFLCPAPSRDSTISEYRDTCPH

SEQ ID NO: 49 - protein, 261 aa, synthetic construct
MAENPYERGPDPTEASIEASRGPFAISQVSVPSGSGSGFGGGTIYYPTDTSQGTFGAVAISPGFTATEASIAWLGPRLASQGFVVITIDTNSRYDQPDARADQLLAALDYLTQKSSVRSRIDPNRLAVMGHSMGGGGTLEAAENRPSLKAAIPLAPWNTNKNWSSVRVPTMIIGAQNDTIAPVGSHAEPFYNSLPASPEKAYLELKGADHFAPTSPNTTIAKYSISWLKRFVDDDTRYDQFLCPAPSSDSAISEYRDTCPH

SEQ ID NO: 50 - protein, 261 aa, synthetic construct
MAENPYERGPAPTESSIEASRGPFAVSQTSVSSLSVSGFGGGTIYYPTSTSEGTFGAVAISPGYTATQSSIAWLGPRLASQGFVVITIDTNSRYDQPASRGDQLLAALDYLTQRSSVRSRIDPSRLAVMGHSMGGGGTLEAAKDRPSLKAAIPLTPWNTDKNWSSVRVPTLIIGAENDTIAPVSSHAEPFYNSLPSSPEKAYLELNGASHFAPNSSNTTIAKYSISWLKRFVDNDTRYEQFLCPAPSPDSTISEYRDTCPH

SEQ ID NO: 51 - protein, 261 aa, synthetic construct
MAENPYERGPDPTESSIEATRGPFAVSQTSVSSLSVSGFGGGTIYYPTSTSEGTFGAVAISPGYTATQSSIAWLGPRLASQGFVVFTIDTNTRYDQPASRGDQLLAALDYLTQRSSVRSRIDASRLAVMGHSMGGGGTLEAAKDRPSLQAAIPLTPWNLDKNWSEVRVPTLIIGAENDTVAPVSSHSEPFYNSLPSSPDKAYLELNGASHFAPNSSNTTIAKYSISWLKRFVDDDTRYEQFLCPAPSPDSAISEYRDTCPH

SEQ ID NO: 52 - protein, 261 aa, synthetic construct
MQSNPYQRGPDPTRASLEASDGPFSVSTTSVSSLSVSGFGGGTIYYPTSTSSGTFGAVAISPGYTATQSSIAWLGRRLASHGFVVITIDTNSRFDQPASRASQLLAALNYLTNSSSVRSRVDASRLAVAGHSMGGGGTLEAAEDNPSLKAAVPLTPWNTDKNWSEVRVPTLIIGAENDTVAPVSSHAIPFYNSLPSSTEKAYVELNGASHFAPNSSNTTISKYSISWMKRFVDNDTRYSQFLCGAPNQDSAISDYRTNCPH

SEQ ID NO: 53 - protein, 261 aa, synthetic construct
MQANPYQRGPDPTSASLEASSGPFSVSTSSVSSLSASGFGGGTIYYPTTTSSGTYGAVAISPGYTATQSSIAWLGRRLASHGFVVITIDTNSTFDQPDSRATQLMAALNYVVNSSTVRSRVDASRLAVMGHSMGGGGTLIAAEDNPSLKAAIPLTPWHTSKNFSSVRVPTLIIGAENDTIAPVSSHAKPFYNSLPSSTSKAYVELNGASHFAPNSSNTPIGKYSISWMKRFVDNDTRYSPFLCGAPHQGAAISEYRDNCPY

SEQ ID NO: 54 - protein, 261 aa, synthetic construct
MADNPYERGPDPTESSIEASRGPFAVSQTSVSSLSVSGFGGGTIYYPTDTSEGTFGAVAISPGYTASQSSIAWLGPRLASQGFVVITIDTNSRYDQPDSRGRQLLAALDYLTQRSSVRSRIDPNRLAVMGHSMGGGGTLEAAEDRPSLKAAIPLTPWHTDKNWSEVRVPTMIIGAENDTIAPVSSHAEPFYNSLPSSPEKAYLELNGASHFAPNTSNTTIAKYSISWLKRFVDDDTRYDQFLCPAPSPDSAISEYRDTCPH

SEQ ID NO: 55 - protein, 261 aa, synthetic construct
MAANPYERGPNPTDALLEARSGPFSVSEENVSRLSASGFGGGTIYYPRENSSNTYGAVAISPGYTGTEASIAWLGERIASHGFVVITIDTITTLDQPDSRAEQLNAALNHMINRSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHVAPNIPNKIIGKYSVAWLKRFVDNDTRYTQFLCPGPRDGGEVEEYRSTCPF

SEQ ID NO: 56 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELDGATHVAPNIPNKIIGKYSVAWLKRFVDNDTRYTQFLCPGPRDGGEVEEYRSTCPF

SEQ ID NO: 57 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALNYMINRSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHVAPNIPNKIIGKYSVAWLKRFVDNDTRYTQFLCPGPRDGGEVEEYRSTCPF

SEQ ID NO: 58 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALNYMINRSTVRSRIDSSRLAVMGHSMGGGGTLRLASQRPDLKAAIPLTPWHLNKNWSSVRVPTLIIGADLDTIAPVATHAKPFYNSLPSSISKAYLELDGATHVAPNIPNKIIGKYSVAWLKRFVDNDTRYTQFLCPGPRDGGEVEEYRSTCPF

SEQ ID NO: 59 - protein, 261 aa, synthetic construct
MAANPYERGPNPTDALLEARSGPFSVSEENVSRLSASGFGGGTIYYPRENSSNTYGAVAISPGYTGTEASIAWLGERIASHGFVVITIDTITTLDQPDSRAEQLNAALNHMINRSTVRSRIDSSRLAVMGHSMGGGGTLRLASQRPDLKAAIPLTPWHLNKNWSSVTVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 60 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 61 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALNYMINRSTVRSRIDSSRLAVMGHSMGGGGTLRLASQRPDLKAAIPLTPWHLNKNWSSVTVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 62 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALNYMINRSTVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 63 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALNYMINRSTVRSRIDSSRLAVMGHSMGGGGTLRLASQRPDLKAAIPLTPWHLNKNWSSVTVPTLIIGACLDTIAPVATHAKPFYNSLPSSISKAYLELDGATHFAPNIPNKIIGKYSVAWLKRFVDNDTRYTQFLCPGPRDGGEVCEYRSTCPF

SEQ ID NO: 64 - protein, 261 aa, synthetic construct
MAANPYERGPNPTDALLEARSGPFSVSEENVSRLSASGFGGGTIYYPRENSSNTYGAVAISPGYTGTEASIAWLGERIASHGFVVITIDTITTLDQPDSRAEQLNAALNHMINRSTVRSRIDSSRLAVMGHSMGGGGTLRLASQRPDLKAAIPLTPWHLNKNWSSVTVPTLIIGACLDTIAPVATHAKPFYNSLPSSISKAYLELCGATHFAPNIPNKIIGKYSVAWLKRFVDNDTRYTQFLCPGPRDGGEVEEYRSTCPF

SEQ ID NO: 65 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGACLDTIAPVATHAKPFYNSLPSSISKAYLELCGATHFAPNIPNKIIGKYSVAWLKRFVDNDTRYTQFLCPGPRDGGEVEEYRSTCPF

SEQ ID NO: 66 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALNYMINRSTVRSRIDSSRLAVMGHSMGGGGTLRLASQRPDLKAAIPLTPWHLNKNWSSVTVPTLIIGACLDTIAPVATHAKPFYNSLPSSISKAYLELCGATHFAPNIPNKIIGKYSVAWLKRFVDNDTRYTQFLCPGPRDGGEVEEYRSTCPF

SEQ ID NO: 67 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALNYMINRSTVRSRIDSSRLAVMGHSMGGGGTLRLASQRPDLKAAIPLTPWHLNKNWSSVRVPTLIIGACLDTIAPVATHAKPFYNSLPSSISKAYLELCGATHFAPNIPNKIIGKYSVAWLKRFVDNDTRYTQFLCPGPRDGGEVEEYRSTCPF

SEQ ID NO: 68 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGARLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 69 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALNYMINRSTVRSRIDSSRLAVMGHSMGGGGTLRLASQRPDLKAAIPLTPWHLNKNWSSVTVPTLIIGARLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 70 - protein, 261 aa, synthetic construct
MAANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALNYMINRSTVRSRIDSSRLAVMGHSMGGGGTLRLASQRPDLKAAIPLTPWHLNKNWSSVRVPTLIIGARLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 71 - protein, 260 aa, synthetic construct
AANPYERGPNPTDALLEARSGPFSVSEERASRLSADGFGGGTIYYPRENSENTYGAVAISPGYTGTQASMAWLGERIASHGFVVITIDTNTTLDQPDSRARQLNAALDHMINDSAVRSRIDSSRLAVMGHSMGGGGTLRLASQRPDLKAAIPLTPWHLNKNWSNVRVPTLIIGADLDTIAPVLTHAKPFYNSLPTSISKAYLELCGATHFAPNIPNKIIGKYSVAWLKRFVDNDTRYTQFLCPGPRDGGEVCEYRSTGPF

SEQ ID NO: 72 - protein, 260 aa, synthetic construct
AANPYERGPNPTDALLEARSGPFSVSEERASRLSADGFGGGTIYYPRENSENTYGAVAISPGYTGTQASMAWLGERIASHGFVVITIDTNTTLDQPDSRARQLNAALNHMINDSAVRSRIDSSRLAVMGHSMGGGGTLRLASQRPDLKAAIPLTPWHLNKNWSSVRVPTLIIGADLDTIAPVLTHAKPFYNSLPTSISKAYLELCGATHFAPNIPNKIIGKYSVAWLKRFVDNDTRYTQFLCPGPRDGGEVCEYRSTGPF

SEQ ID NO: 73 - protein, 260 aa, synthetic construct
AANPYERGPNPTNALLEARSGPFSVSEENVSRLSADGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 74 - protein, 260 aa, synthetic construct
AANPYERGPNPTDALLEARSGPFSVSEENVSRLSADGFGGGTIYYPRENSENTYGAVVISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 75 - protein, 260 aa, synthetic construct
AANPYERGPNPTDALLEARSGPFSVSEERVSRLSADGFGGGTIYYPRENSENTYGAVAISPGYTGTQASMAWLGERIASHGFVVITIDTNTTLDQPDSRARQLNAALDHMINDSAVRSRIDSSRLAVMGHSMGGGGTLRLASQRPDLKAAIPLTPWHLNKNWSSVRVPTLIIGADLDTIAPVLTHAKPFYNSLPTSISKAYLELCGATHFAPNIPNKIIGKYSVAWLKRFVDNDTRYTQFLPPGPRDGGEVCEYRSTGPF

SEQ ID NO: 76 - protein, 260 aa, synthetic construct
MANPYERGPNPTEALLEARSGPFSVSEERASRLGADGFGGGTIYYPRENSENTYGAVAISPGYTGTQASVAWLGERIASHGFVVITIDTNTTLDQPDSRARQLNAALDYMLNDSAVRSRIDSSRLAVMGHSMGGGGTLRLASQRPDLKAAIPLTPWHLNKSWSNVQVPTLIIGADLDTIAPVLTHAEPFYNSIPTSTSKAYLELDGATHFAPNITNKTIGMYSVAWLKRFVDEDTRYTQFLCPGPRTGSDVEEYRSTCPF

SEQ ID NO: 77 - protein, 260 aa, synthetic construct
AANPYERGPNPTDALLEARSGPFSVSEETVSRLSASGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGDVCEYRSTCPF

SEQ ID NO: 78 - protein, 260 aa, synthetic construct
AANPYERGPNPTDALLEARSGPFSVSEENVSRLSADGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKTWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 79 - protein, 260 aa, synthetic construct
AANPYERGPNPTDALLEARRGPFSVSEENVSRLSADGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 80 - protein, 260 aa, synthetic construct
AANPYERGPNPTDALLEARSGPFSVSEERASRLGADGFGGGTIYYPRENSENTYGAVAISPGYTGTQASMAWLGERIASHGFVVITIDTNTTLDQPDSRARQLNAALDHMINDSAVRSRIDSSRLAVMGHSMGGGGTLRLASQRPDLKAAIPLTPWHLNKNWSSVRVPTLIIGADLDTIAPVLTHAKPFYNSLPTSISKAYLELCGATHFAPNIPNKIIGKYSVAWLKRFVDNDTRYTQFLPPGPRTGGEVCEYRSTGPF

SEQ ID NO: 81 - protein, 260 aa, synthetic construct
MANPYERGPNPTDALLEARSGPFSVSEERASRLGADGFGGGTIYYPRENSENTYGAVAISPGYTGTQASMAWLGERIASHGFVVITIDTNTTLDQPDSRARQLNAALDHMINDSAVRSRIDSSRLAVMGHSMGGGGTLRLASQRPDLKAAIPLTPWHLNKNWSNVRVPTLIIGADLDTIAPVLTHAKPFYNSLPTSISKAYLELDGATHFAPNIPNKIIGKYSVAWLKRFVDNDTRYTQFLPPGPRTGGEVSEYRSTGPF

SEQ ID NO: 82 - protein, 260 aa, synthetic construct
MANPYERGPNPTDALLEARSGPFSVSEERASRLGADGFGGGTIYYPRENSENTYGAVAISPGYTGTQASVAWLGERIASHGFVVITIDTNTTLDQPDSRARQLNAALDYMINDSAVRSRIDSSRLAVMGHSMGGGGTLRLASQRPDLKAAIPLTPWHLNKNWSSVTVPTLIIGADLDTIAPVLTHARPFYNSLPTSISKAYLELDGATHFAPNIPNKIIGKYSVAWLKRFVDNDTRYTQFLCPGPRDGGEVEEYRSTCPF

SEQ ID NO: 83 - protein, 260 aa, synthetic construct
MANPYERGPNPTEALLEARSGPFSVSEERASRLGADGFGGGTIYYPRENSDNTYGAVAISPGYTGTQASVAWLGERIASHGFVVITIDTNTTLDQPDSRARQLNAALDYMLNDSAVRSRIDSSRLAVMGHSMGGGGTLRLASQRPDLKAAIPLTPWHLNKSWSNVQVPTLIIGADLDTIAPVLTHAEPFYNSIPTSTSKAYLELDGATHFAPNITNKTIGMYSVAWLKRFVDEDTRYTQFLCPGPRTGSDVEEYRSTCPF

SEQ ID NO: 84 - protein, 260 aa, synthetic construct
AANPYERGPNPTDALLEARSGPFSVSEENVSRLSADGFGGGTIYYPRENSENTYGAVAISPGYTGTQASMAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 85 - protein, 260 aa, synthetic construct
AANPYERGPNPTDALLEARSGPFSVSEENVSRLSADGFGGGTIYYPRENSENTYGAVAISPGYTGTQASIAWLGERIASHGFVVITIDTNTTLDQPDSRAKQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 86 - protein, 260 aa, synthetic construct
AANPYERGPNPTDALLEARSGPFSVSEENVSRLSADGFGGGTIYYPRENSENTYGAVAISPGYTGTQASIAWLGERIASHGFVVITIDTNTTLDQPDSRARQLNAALDYMINDSAVRSRIDSSRLGVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 87 - protein, 260 aa, synthetic construct
MANPYERGPNPTDALLEARSGPFSVSEERASRLGADGFGGGTIYYPRENSDNTYGAVAISPGYTGTQASMAWLGERIASHGFVVITIDTNTTLDQPDSRARQLNAALDHMINDSTVRSRIDSSRLAVMGHSMGGGGTLRLASQRPDLKAAIPLTPWHLNKNWSNVRVPTLIIGADLDTIAPVLTHAKPFYNSLPTSISKAYLELCGATHFAPNIPNKTIGKYSVAWLKRFVDNDTRYTQFLPPGPRTGGEVCEYRSTGPF

SEQ ID NO: 88 - protein, 260 aa, synthetic construct
AANPYERGPNPTDALLEARSGPFSVSEERASRLSADGFGGGTIYYPRENSENTYGAVAISPGYTGTQASMAWLGERIASHGFVVITIDTNTTLDQPDSRARQLNAALDHMINDSAVRSRIDSSRLAVMGHSMGGGGTLRLASQRPDLKAAIPLTPWHLNKNWSSVRVPTLIIGADLDTIAPVLTHAKPFYNSLPTSISKAYLELCGATHFAPNIPNKIIGKYSVAWLKRFVDNDTRYTQFLPPGPRDGGEVCEYRSTGPF

SEQ ID NO: 89 - protein, 260 aa, synthetic construct
MANPYERGPNPTEALLEARSGPFSVSEERASRLGADGFGGGTIYYPRENSENTYGAVAISPGYTGTQASVAWLGERIASHGFVVITIDTNTTLDQPDSRARQLNAALDYMLNDSAVRSRIDSSRLAVMGHSMGGGGTLRLASQRPDLKAAIPLTPWHLNKSWSNVTVPTLIIGADLDTIAPVLTHAEPFYNSIPTSTSKAYLELDGATHFAPNITNKTIGMYSVAWLKRFVDEDTRYTQFLCPGPRTGSDVEEYRSTCPF

SEQ ID NO: 90 - protein, 260 aa, synthetic construct
MANPYERGPNPTEALLEARSGPFSVSEERASRLGADGFGGGTIYYPRENSSNTYGAVAISPGYTGTQASVAWLGERIASHGFVVITIDTNTTLDQPDSRARQLNAALDYMLNDSAVRSRIDSSRLAVMGHSMGGGGTLRLASQRPDLKAAIPLTPWHLNKSWSNVQVPTLIIGADLDTIAPVLTHAEPFYNSIPTSTSKAYLELDGATHFAPNITNKTIGMYSVAWLKRFVDEDTRYTQFLCPGPRTGSDVEEYRSTCPF

SEQ ID NO: 91 - protein, 260 aa, synthetic construct
AENPYERGPDPTESSIEASRGPFAVSETSVSRLSVSGFGGGTIYYPTSTSEGTFGAVAISPGYTATQSSIAWLGPRLASQGFVVITIDTNSRYDQPDSRARQLLAALDYLTNSSSVRSRIDPSRLAVMGHSMGGGGTLQAAEDRPSLKAAIPLTPWHTDKNWSSVRVPTLIIGAENDTVAPVSSHAKPFYNSLPSSPEKAYLELNGASHFAPNSSNTTIAKYSIAWLKRFVDDDTRYEQFLCPAPSTDSAISEYRSTCPY

SEQ ID NO: 92 - protein, 260 aa, synthetic construct
AANPYERGPNPTDALLEARSGPFSVSEENVSRLSASGFGGGTIYYPRENSENTYGAVAISPGYTGTQASMAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGDVCEYRSTCPF

SEQ ID NO: 93 - protein, 260 aa, synthetic construct
AANPYERGPNPTDALLEARSGPFSVSEESVSRLSASGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGDVCEYRSTCPF

SEQ ID NO: 94 - protein, 260 aa, synthetic construct
AANPYERGPNPTDALLEARSGPFSVSEENVSRLSADGFGGGTIYYPRENSENTYGAVAISPGYTGTQASIAWLGERIASHGFVVITIDTNTTLDQPDSRARQLNAALDYMINDSAVRSMIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 95 - protein, 260 aa, synthetic construct
AANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWNSVRVPTLIIGADLDTIAPVATHAKPFYNSLPSSISKAYLELCGATHFAPNIPNKIIGKYSVAWLKRFVDNDTRYTQFLCPGPRDGGDVCEYRSTCPF

SEQ ID NO: 96 - protein, 260 aa, synthetic construct
AANPYERGPNPTDALLEARSGPFSVSEENVSRLSADGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGDVCEYRSTCPF

SEQ ID NO: 97 - protein, 260 aa, synthetic construct
AANPYERGPNPTNALLEARSGPFSVSEENVSRLSASGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGDVCEYRSTCPF

SEQ ID NO: 98 - protein, 260 aa, synthetic construct
AANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGGTHFAPNIPNKIIGKYSVAWLKRFVDNDTRYTQFLCPGPRDGGDVCEYRSTCPF

SEQ ID NO: 99 - protein, 260 aa, synthetic construct
QANPYERGPNPTDALLEARSGPFSVSEENVSRLSADGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 100 - protein, 260 aa, synthetic construct
AANPYERGPDPTESSLEASSGPFAVSETSVSRLSVSGFGGGTIYYPTSTSSGTYGAVAISPGYTATQSSIAWLGPRLASHGFVVITIDTNSRFDQPDSRARQLLAALNYLVNSSSVRSRIDSSRLAVMGHSMGGGGTLRAAEDRPSLKAAIPLTPWHTDKNWSSVTVPTLIIGAENDTVAPVSSHAKPFYNSLPSSTPKAYLELNGASHFAPNSSNTTIGKYSIAWLKRFVDNDTRYSQFLCPAPSDDSAISEYRSTCPY

SEQ ID NO: 101 - protein, 260 aa, synthetic construct
MANPYERGPNPTDALLEARSGPFSVSEERASRLGADGFGGGTIYYPRENSENTYGAVAISPGYTGTQASVAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVLTHARPFYNSLPTSISKAYLELDGATHFAPNIPNKIIGKYSVAWLKRFVDNDTRYTQFLCPGPRDGGEVEEYRSTCPF

SEQ ID NO: 102 - protein, 260 aa, synthetic construct
AANPYERGPNPTDALLEARSGPFSVSEENVSRLSADGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWNSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 103 - protein, 260 aa, synthetic construct
AANPYERGPNPTDALLEARSGPFSVSEENVSRLSADGFGGGTIYYPRENSTNTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 104 - protein, 260 aa, synthetic construct
AANPYERGPNPTDALLEARSGPFSVSEERVSRLSADGFGGGTIYYPRENSENTYGAVAISPGYTGTQASMAWLGERIASHGFVVITIDTNTTLDQPDSRARQLNAALNHMINDSAVRSRIDSSRLAVMGHSMGGGGTLRLASQRPDLKAAIPLTPWHLNKNWSSVRVPTLIIGADLDTIAPVLTHAKPFYNSLPTSISKAYLELCGATHFAPNIPNKIIGKYSVAWLKRFVDNDTRYTQFLPPGPRDGGEVCEYRSTGPF

SEQ ID NO: 105 - protein, 260 aa, synthetic construct
AENPYERGPDPTESSIEALRGPFAVSEESVSRLSVSGFGGGTIYYPTDTSEGTFGAVAISPGYTGTQSSMAWLGPRIASQGFVVFTIDTNTTYDQPDSRARQLQAALDYLVEDSSVRDRIDPNRLGVMGHSMGGGGTLRAAEDRPDLKAAIPLTPWHLNKNWSSVRVPTLIIGAENDTIAPVRTHAEPFYESLPSSLDKAYLELDGASHFAPNISNTTIAKYSISWLKRFVDDDTRYEQFLCPPPRTGSEISEYRSTCPF

SEQ ID NO: 106 - protein, 260 aa, synthetic construct
AANPYERGPNPTEALLEARSGPFSVSEERASRLGADGFGGGTIYYPRENSDNTYGAVAISPGYTGTQASVAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVLTHAEPFYNSLPTSISKAYLELDGATHFAPNITNKTIGKYSVAWLKRFVDEDTRYTQFLCPGPRTGSDVEEYRSTCPF

SEQ ID NO: 107 - protein, 260 aa, synthetic construct
AANPYERGPNPTDALLEARSGPFSVSEERASRLGADGFGGGTIYYPRENSDNTYGAVAISPGYTGTQASVAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVLTHAKPFYNSLPTSISKAYLELDGATHFAPNIPNKIIGKYSVAWLKRFVDNDTRYTQFLCPGPRDGGEVEEYRSTCPF

SEQ ID NO: 108 - protein, 260 aa, synthetic construct
AANPYERGPNPTDALLEARSGPFSVSEERVSRLSADGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 109 - protein, 260 aa, synthetic construct
AANPYERGPNPTDALLEARSGPFSVSEENVSRLSADGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGGTHFAPNIPNKIIGKYSVAWLKRFVDNDTRYTQFLCPGPRDGGEVCEYRSTCPF

SEQ ID NO: 110 - protein, 260 aa, synthetic construct
AANPYERGPNPTDALLEARSGPFSVSEENVSRLSADGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGREVCEYRSTCPF

SEQ ID NO: 111 - protein, 260 aa, synthetic construct
AANPYERGPNPTDALLEARSGPFSVSEENVSRLSADGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFTPNIPNKIIGKYSVAWLKRFVDNDTRYTQFLCPGPRDGGEVCEYRSTCPF

SEQ ID NO: 112 - protein, 260 aa, synthetic construct
AANPYERGPNPTDALLEARSGPFSVSEERVSRLGADGFGGGTIYYPRENSENTYGAVAISPGYTGTQASMAWLGERIASHGFVVITIDTNTTLDQPDSRARQLNAALDHMINDSAVRSRIDSSRLAVMGHSMGGGGTLRLASQRPDLKAAIPLTPWHLNKNWSSVRVPTLIIGADLDTIAPVLTHAKPFYNSLPTSISKAYLELCGATHFAPNIPNKIIGKYSVAWLKRFVDNDTRYTQFLPPGPRDGGEVCEYRSTGPF

SEQ ID NO: 113 - protein, 260 aa, synthetic construct
AANPYERGPDPTESSLEASSGPFSVSETSVSRLSVSGFGGGTIYYPTDTSSGTYGAVAISPGYTATQSSIAWLGPRLASHGFVVITIDTNSTFDQPDSRARQLMAALDYLVNSSSVRSRIDSSRLAVMGHSMGGGGTLRAAEDNPSLKAAIPLTPWHTDKNWSSVRVPTLIIGAENDTVAPVSSHAKPFYNSLPSSTPKAYLELNGASHFAPNSSNTTIGKYSVSWLKRFVDNDTRYSQFLCPAPHDDSAISEYRSTCPY

SEQ ID NO: 114 - protein, 260 aa, synthetic construct
AANPYERGPNPTEALLEARSGPFSVSEERASRLGADGFGGGTIYYPRENSDNTYGAVAISPGYTGTQASVAWLGERIASHGFVVITIDTNTTLDQPDSRARQLNAALDYMLNDSAVRSRIDSSRLAVMGHSMGGGGTLRLASQRPDLKAAIPLTPWHLNKSWSNVQVPTLIIGADLDTIAPVLTHAEPFYNSIPTSTSKAYLELDGATHFAPNITNKTIGMYSVAWLKRFVDEDTRYTQFLCPGPRTGSDVEEYRSTCPF

SEQ ID NO: 115 - protein, 260 aa, synthetic construct
AANPYERGPNPTDALLEARSGPFSVSEESVSRLSADGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 116 - protein, 260 aa, synthetic construct
AANPYERGPNPTDALLEARSGPFSVSEENVSRLSASGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKTWSSVRVPTLIIGADLDTIAPVATHAKPFYNSLPSSISKAYLELCGATHFAPNIPNKIIGKYSVAWLKRFVDNDTRYTQFLCPGPRDGGDVCEYRSTCPF

SEQ ID NO: 117 - protein, 260 aa, synthetic construct
AANPYERGPNPTDALLEARSGPFTVSEENVSRLSADGFGGGTIYYPRENSENTYGAVAISPGYTGTQASIAWLGERIASHGFVVITIDTNTTLDQPDSRARQLNAALDYMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 118 - protein, 260 aa, synthetic construct
AANPYERGPNPTDALLEARSGPFSVSEENVSRLSADGFGGGTIYYPRENSENTYGAVAISPGYTGTQASIAWLGERIASHGFVVITIDTNTTLDQPDSRARQLNAALDHMINDSAVRSRIDSSRLAVMGHSMGGGGTLRLASQRPDLKAAIPLTPWHLNKNWSSVRVPTLIIGADLDTIAPVATHAKPFYNSLPSSISKAYLELCGATHFAPNIPNKIIGKYSVAWLKRFVDNDTRYTQFLCPGPRDGGEVCEYRSTCPF

SEQ ID NO: 119 - protein, 253 aa, synthetic construct
GPXPTXXXXEAXXGPFXXXXXXXXXXXXXGFGGGTIYYPXXXSXXXXGAVXISPGXTXXXXSXAWLGXRXASXGFVVXTIDTXXXXDQPXXRXXQLXAALXXXXXXSXVRXXXDXXRLXVXGHSMGGGGTLXXAXXXPXLXAAXPLXXWXXXKXXXXVXVPTXXIGXXXDTXAPVXXHXXPFYXSXPXXXXKAYXELXXXXHXXPXXXNXXXXXYXXXWXKRFXDXDTRYXXFLXXXPXXXXXXXXYRXXXPX

SEQ ID NO: 120 - protein, 4 aa, synthetic construct
MAAN

SEQ ID NO: 121 - protein, 4 aa, synthetic construct
MAEN

SEQ ID NO: 122 - protein, 4 aa, synthetic construct
MQAN

SEQ ID NO: 123 - protein, 4 aa, synthetic construct
MADN

SEQ ID NO: 124 - protein, 4 aa, synthetic construct
MQSN